SEQUENCE LISTING (40 sequences)

SEQ ID NO: 1 - DNA, 3002 bp, Artificial Sequence
  Synthesized Acidothermus cellulolyticus 11B E I beta-1,4-endoglucanase precursor
ggatccacgttgtacaaggtcacctgtccgtcgttctggtagagcggcgggatggtcacccgcacgatctctcctttgttgatgtcgacggtcacgtggttacggtttgcctcggccgcgattttcgcgctcgggcttgctccggctgtcgggttcggtttggcgtggtgtgcggagcacgccgagcgatcccaatgagggcaagggcaagagcggagccgatggcacgtcgggtggccgatggggtacgccgatggggcgtggcgtccccgccgcggacagaaccggatgcggaataggtcacggtgcgacatgttgccgtaccgcggacccggatgacaagggtgggtgcgcgggtcgcctgtgagctgccggctggcgtctggatcatgggaacgatcccaccattccccgcaatcgacgcgatcgggagcagggcggcgcgagccggaccgtgtggtcgagccggacgattcgcccatacggtgctgcaatgcccagcgccatgttgtcaatccgccaaatgcagcaatgcacacatggacagggattgtgactctgagtaatgattggattgccttcttgccgcctacgcgttacgcagagtaggcgactgtatgcggtaggttggcgctccagccgtgggctggacatgcctgctgcgaactcttgacacgtctggttgaacgcgcaatactcccaacaccgatgggatcgttcccataagtttccgtctcacaacagaatcggtgcgccctcatgatcaacgtgaaaggagtacgggggagaacagacgggggagaaaccaacgggggattggcggtgccgcgcgcattgcggcgagtgcctggctcgcgggtgatgctgcgggtcggcgtcgtcgtcgcggtgctggcattggttgccgcactcgccaacctagccgtgccgcggccggctcgcgccgcgggcggcggctattggcacacgagcggccgggagatcctggacgcgaacaacgtgccggtacggatcgccggcatcaactggtttgggttcgaaacctgcaattacgtcgtgcacggtctctggtcacgcgactaccgcagcatgctcgaccagataaagtcgctcggctacaacacaatccggctgccgtactctgacgacattctcaagccgggcaccatgccgaacagcatcaatttttaccagatgaatcaggacctgcagggtctgacgtccttgcaggtcatggacaaaatcgtcgcgtacgccggtcagatcggcctgcgcatcattcttgaccgccaccgaccggattgcagcgggcagtcggcgctgtggtacacgagcagcgtctcggaggctacgtggatttccgacctgcaagcgctggcgcagcgctacaagggaaacccgacggtcgtcggctttgacttgcacaacgagccgcatgacccggcctgctggggctgcggcgatccgagcatcgactggcgattggccgccgagcgggccggaaacgccgtgctctcggtgaatccgaacctgctcattttcgtcgaaggtgtgcagagctacaacggagactcctactggtggggcggcaacctgcaaggagccggccagtacccggtcgtgctgaacgtgccgaaccgcctggtgtactcggcgcacgactacgcgacgagcgtctacccgcagacgtggttcagcgatccgaccttccccaacaacatgcccggcatctggaacaagaactggggatacctcttcaatcagaacattgcaccggtatggctgggcgaattcggtacgacactgcaatccacgaccgaccagacgtggctgaagacgctcgtccagtacctacggccgaccgcgcaatacggtgcggacagcttccagtggaccttctggtcctggaaccccgattccggcgacacaggaggaattctcaaggatgactggcagacggtcgacacagtaaaagacggctatctcgcgccgatcaagtcgtcgattttcgatcctgtcggcgcgtctgcatcgcctagcagtcaaccgtccccgtcggtgtcgccgtctccgtcgccgagcccgtcggcgagtcggacgccgacgcctactccgacgccgacagccagcccgacgccaacgctgacccctactgctacgcccacgcccacggcaagcccgacgccgtcaccgacggcagcctccggagcccgctgcaccgcgagttaccaggtcaacagcgattggggcaatggcttcacggtaacggtggccgtgacaaattccggatccgtcgcgaccaagacatggacggtcagttggacattcggcggaaatcagacgattaccaattcgtggaatgcagcggtcacgcagaacggtcagtcggtaacggctcggaatatgagttataacaacgtgattcagcctggtcagaacaccacgttcggattccaggcgagctataccggaagcaacgcggcaccgacagtcgcctgcgcagcaagttaatacgtcggggagccgacgggagggtccggaccgtcggttccccggcttccacctatggagcgaacccaacaatccggacggaactgcaggtaccagagaggaacgacacgaatgcccgccatctcaaaacggctgcgagccggcgtcctcgccggggcggtgagcatcgcagcctccatcgtgccgctggcgatgcagcatcctgccatcgccgcgacgcacgtcgacaatccctatgcgggagcgaccttcttcgtcaacccgtactgggcgcaagaagtacagagcgaacggcgaaccagaccaatgccactctcgcagcgaaaatgcgcgtcgtttccacatattcgacggccgtctggatggaccgcatcgctgcgatcaacggcgtcaacggcggacccggcttgacgacatatctggacgccgccctctcccagcagcagggaaccacccctgaagtcattgagattgtatctacgatctgccgg

SEQ ID NO: 2 - DNA, 1527 bp, Artificial Sequence
  Synthesized EndoglucanaseI (EGI, Cel7B) Trichoderma reesei/Hypocrea jecorina
ttgtcccaaaatggcgccctcagttacactgccgttgaccacggccatcctggccattgcccggctcgtcgccgcccagcaaccgggtaccagcacccccgaggtccatcccaagttgacaacctacaagtgtacaaagtccggggggtgcgtggcccaggacacctcggtggtccttgactggaactaccgctggatgcacgacgcaaactacaactcgtgcaccgtcaacggcggcgtcaacaccacgctctgccctgacgaggcgacctgtggcaagaactgcttcatcgagggcgtcgactacgccgcctcgggcgtcacgacctcgggcagcagcctcaccatgaaccagtacatgcccagcagctctggcggctacagcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagatggatatcctggagggcaactcgagggcgaatgccttgacccctcactcttgcacggccacggcctgcgactctgccggttgcggcttcaacccctatggcagcggctacaaaaggtgagcctgatgccactactacccctttcctggcgctctcgcggttttccatgctgacatggttttccagctactacggccccggagataccgttgacacctccaagaccttcaccatcatcacccagttcaacacggacaacggctcgccctcgggcaaccttgtgagcatcacccgcaagtaccagcaaaacggcgtcgacatccccagcgcccagcccggcggcgacaccatctcgtcctgcccgtccgcctcagcctacggcggcctcgccaccatgggcaaggccctgagcagcggcatggtgctcgtgttcagcatttggaacgacaacagccagtacatgaactggctcgacagcggcaacgccggcccctgcagcagcaccgagggcaacccatccaacatcctggccaacaaccccaacacgcacgtcgtcttctccaacatccgctggggagacattgggtctactacgaactcgactgcgcccccgcccccgcctgcgtccagcacgacgttttcgactacacggaggagctcgacgacttcgagcagcccgagctgcacgcagactcactgggggcagtgcggtggcattgggtacagcgggtgcaagacgtgcacgtcgggcactacgtgccagtatagcaacgactgttcgtatccccatgcctgacgggagtgattttgagatgctaaccgctaaaatacagactactcgcaatgcctttagagcgttgact

SEQ ID NO: 3 - DNA, 1849 bp, Artificial Sequence
  Synthesized EndoglucanaseII (EGII, Cel5A) Trichoderma reesei/Hypocrea jecorina
tgccatttctgacctggataggttttcctatggtcattcctataagagacacgctctttcgtcggcccgtagatatcagattggtattcagtcgcacagacgaaggtgagttgatcctccaacatgagttctatgagccccccccttgcccccccccgttcaccttgacctgcaatgagaatcccaccttttacaagagcatcaagaagtattaatggcgctgaatagcctctgctcgataatatctccccgtcatcgacaatgaacaagtccgtggctccattgctgcttgcagcgtccatactatatggcggcgccgtcgcacagcagactgtctggggccagtgtggaggtattggttggagcggacctacgaattgtgctcctggctcagcttgttcgaccctcaatccttattatgcgcaatgtattccgggagccactactatcaccacttcgacccggccaccatccggtccaaccaccaccaccagggctacctcaacaagctcatcaactccacccacgagctctggggtccgatttgccggcgttaacatcgcgggttttgactttggctgtaccacagagtgagtacccttgtttcctggtgttgctggctggttgggcgggtatacagcgaagcggacgcaagaacaccgccggtccgccaccatcaagatgtgggtggtaagcggcggtgttttgtacaactacctgacagctcactcaggaaatgagaattaatggaagtcttgttacagtggcacttgcgttacctcgaaggtttatcctccgttgaagaacttcaccggctcaaacaactaccccgatggcatcggccagatgcagcacttcgtcaacgaggacgggatgactattttccgcttacctgtcggatggcagtacctcgtcaacaacaatttgggcggcaatcttgattccacgagcatttccaagtatgatcagcttgttcaggggtgcctgtctctgggcgcatactgcatcgtcgacatccacaattatgctcgatggaacggtgggatcattggtcagggcggccctactaatgctcaattcacgagcctttggtcgcagttggcatcaaagtacgcatctcagtcgagggtgtggttcggcatcatgaatgagccccacgacgtgaacatcaacacctgggctgccacggtccaagaggttgtaaccgcaatccgcaacgctggtgctacgtcgcaattcatctctttgcctggaaatgattggcaatctgctggggctttcatatccgatggcagtgcagccgccctgtctcaagtcacgaacccggatgggtcaacaacgaatctgatttttgacgtgcacaaatacttggactcagacaactccggtactcacgccgaatgtactacaaataacattgacggcgccttttctccgcttgccacttggctccgacagaacaatcgccaggctatcctgacagaaaccggtggtggcaacgttcagtcctgcatacaagacatgtgccagcaaatccaatatctcaaccagaactcagatgtctatcttggctatgttggttggggtgccggatcatttgatagcacgtatgtcctgacggaaacaccgactagcagtggtaactcatggacggacacatccttggtcagctcgtgtctcgcaagaaagtagcactctgagctgaatgcagaagcctcgccaacgtttgtatctcgctatcaaacatagtagctactctatgaggctgtctgttctcgatttcagctttatatagtttcatcaaacagtacatattccctctgtggccacgcaaaaaaaaaaaaaaaaa

SEQ ID NO: 4 - DNA, 1373 bp, Artificial Sequence
  Synthesized EndoglucanaseII (EGII, Cel5A) Penicillium janthinellum
atgatggctcaggacaagcgcactctgttggcactccttgcccttggcggatccgcgctagggcagcaaacggcttggggacaatgtggtggtcagggatggactggggcaaccacatgtgtgtctgggtactactgctccttccagaacaattggtactctcagtgccttcctggcgcggccagcactactagtacctccaccactaccactaccgtgtactcaaccacgttaaagacgacgacgacttcgtcaacttcttccgcgccgacaggcaaagtacgatttgctggtgtcaacattgccggcttcgactttggcgtggtgacaagtggtactcaggacctgtctcaggtcgtcgatgagtccgtggacggtgtcaaccagatgtcacacttcgtcaacgcggataccttcaacattttccgcctgccgactggatggcagttcattgtcaacaacaaccttggcggttctctcgactcgaacaactttggcaagtatgaacaagttggttcagggctgtctctctctggcgcttattgtattgttgatatccacaactacgcccgctggaacggtggcgttatcggacaaggcggtccaaccgatgaccaattcatcagcctctggacgcaattggccactcactacaagagtaacagcagggttatctttggcatcatgaacgagccccatgatctcaacatcgcaacctgggctgccacagtccagaagactgtgactgcgatcagaaacactggagccacgtctcagatgattctgctccctggtacggactacaccagcgctgccaacttcattgagaatggctccggtgcagcgctcctaccggtgaccaacccggatggatccaccacgaacctgatctttgatgttcacaagtacctggatagtgacaacagcggcacccacgccgagtgcgtgaccaacaatgccgacgccttcaacaatcttgcaacctggctgcgctcaaacaagagacaggctctgctttccgagactggtggcggcaacgtccaaagttgtgccacttacatgtgccagcagcttgatatcctcaatgcaaattctgatgtctacctcggctggacctcttggagcgctggaggcttccaggcctcttggaactatgtcctgactgaagtcccagtcaatggtgttgatcaataccttgtccaacagtgcttcgtccccaagtggaagaactgagcgctggtagactggtcatgtatggatccgattttgctacttagagaaggaatctcctccagttagtgcttttgagtctggacaggctggacttgaacagttaggcaatataattctagcaccactcttatgatcaaaaa

SEQ ID NO: 5 - DNA, 1017 bp, Artificial Sequence
  Synthesized EndoglucanaseIII (Cel12A) Trichoderma longibrachiatum
tggccaaatcgtgatcgattgatactcgcatctataagatggcacagatcgactcttgattcacagacatccgtcagccctcaagccgtttgcaagtccacaaacacaagcacaagcatagcgtcgcaatgaagttccttcaagtcctccctgccctcataccggccgccctggcccaaaccagctgtgaccagtgggcaaccttcactggcaacggctacacagtcagcaacaacctttggggagcatcagccggctctggatttggctgcgtgacggcggtatcgctcagcggcggggcctcctggcacgcagactggcagtggtccggcggccagaacaacgtcaagtcgtaccagaactctcagattgccattccccagaagaggaccgtcaacagcatcagcagcatgcccaccactgccagctggagctacagcgggagcaacatccgcgctaatgttgcgtatgacttgttcaccgcagccaacccgaatcatgtcacgtactcgggagactacgaactcatgatctggtaagccataagaagtgaccctccttgatagtttcgactaacaacatgtcttgaggcttggcaaatacggcgatattgggccgattgggtcctcacagggaacagtcaacgtcggtggccagagctggacgctctactatggctacaacggagccatgcaagtctattcctttgtggcccagaccaacactaccaactacagcggagatgtcaagaacttcttcaattatctccgagacaataaaggatacaacgctgcaggccaatatgttcttagtaagtcaccctcactgtgactgggctgagtttgttgcaacgtttgctaacaaaaccttcgtataggctaccaatttggtaccgagcccttcacgggcagtggaactctgaacgtcgcatcctggaccgcatctatcaactaaaactggaaacgtgagatgtggtgggcatacgttattgagcgagggaaaaaaagcattggatcc

SEQ ID NO: 6 - DNA, 1399 bp, Artificial Sequence
  Synthesized EndoglucanaseIV (Cel61A) Trichoderma reesei/Hypocreajecorina
cagatataggctttactgagactcgctttgtttctttcacaaaaaaatgatccagaagctttccaacctccttgtcaccgcactggcggtggctactggcgttgtcggacatggacatattaatgacattgtcatcaacggggtgtggtatcaggcctatgatcctacaacgtttccatacgagtcaaacccccccatagtagtgggctggacggctgccgaccttgacaacggcttcgtttcacccgacgcataccaaaaccctgacatcatctgccacaagaatgctacgaatgccaaggggcacgcgtctgtcaaggccggagacactattctcttccagtgggtgccagttccatggccgcaccctggtcccattgtcgactacctggccaactgcaatggtgactgcgagaccgttgacaagacgacgcttgagttcttcaagatcgatggcgttggtctcctcagcggcggggatccgggcacctgggcctcagacgtgctgatctccaacaacaacacctgggtcgtcaagatccccgacaatcttgcgccaggcaattacgtgctccgccacgagatcatcgcgttacacagcgccgggcaggcaaacggcgctcagaactacccccagtgcttcaacattgccgtctcaggctcgggttctctgcagcccagcggcgttctagggaccgacctctatcacgcgacggaccctggtgttctcatcaacatctacaccagcccgctcaactacatcatccctggacctaccgtggtatcaggcctgccaacgagtgttgcccaggggagctccgccgcgacggccaccgccagcgccactgttcctggaggcggtagcggcccgaccagcagaaccacgacaacggcgaggacgacgcaggcctcaagcaggcccagctctacgcctcccgcaaccacgtcggcacctgctggcggcccaacccagactctgtacggccagtgtggtggcagcggttacagcgggcctactcgatgcgcgccgccagccacttgctctaccttgaacccctactacgcccagtgccttaactagagggcatcacggcgggtctttggagcttcgaggcacacacgcgggctagtgcttcctagaactgaggtagttgttcggggcgaggaggaataatcttttacatatactgtactgaatcttgacgatgtaactcctgatatcactagatgaagtctacgggtatagtgatattgccaagagcagattgttacattatcaagataatctggtcatcatcaatatggttcaggtcccaccacactccttcattccgcttagatagctagttgtccataggtatagcaatgagattcgattcaccattgtcatctactagcc

SEQ ID NO: 7 - DNA, 1124 bp, Artificial Sequence
  Synthesized EndoglucanaseV (Cel45A) Trichoderma reesei/Hypocreajecorina
cgtatcttacacaagggcgctgcaactaattgacttgatcttccatctcgtgtcttgcttgtaaccatcgtgaccatgaaggcaactctggttctcggctccctcattgtaggcgccgtttccgcgtacaaggccaccaccacggcaagtctacatgcttccaggtcacaacgtctgctcaacaacctctaaccgaaaggccagcgctactacgatgggcaggagggtgcttgcggatgcggctcgagctccggcgcattcccgtggcaggtaaacattcgaccttgtctggggcaagggactcgtcacttacatcctctctctctctgcagctcggcatcggcaacggagtctacacggctgccggctcccaggctctcttcgacacggccggagcttcatggtgcggcgccggctgcggtaaatgctaccagctcacctcgacgggccaggcgccctgctccagctgcggcacgggcggtgctgctggccagagcatcatcgtcatggtgaccaacctgtgcccgaacaatgggaacgcgcagtggtgcccggtggtcggcggcaccaaccaatacggctacagctaccatttcgacatcatggcgcagaacgagatctttggagacaatgtcgtcgtcgactttgagcccattgcttgccccgggcaggctgcctctgactgggggacgtgcctctgcgtgggacagcaagagacggatcccacgcccgtcctcggcaacgacacgggctcaactcctcccgggagctcgccgccagcgacatcgtcgagtccgccgtctggcggcggccagcagacgctctatggccagtgtggaggtgccggctggacgggacctacgacgtgccaggccccagggacctgcaaggttcagaaccagtggtactcccagtgtcttccttgagaaggcccaagatagccatgtctctctagcattcttccggcgtcagtctgatctgcctatttaatcaggtcagtcaatatgtatccagagataataaattatgtatattatagcagtactgtatcattgctgctgtctgcctgacttcaatgctgcttcccctattctcgttgcagtagcgttggcgatatggggcag

SEQ ID NO: 8 - DNA, 3189 bp, Artificial Sequence
  Synthesized Endo-1,4--glucanase A (eglA) Aspergillus nidulans
gagctcggtggtagggtgtcgattgcatacagtacttaggtttaggggtggttcaagtacggcagttatttccatttcattctatctcataccatttctctcttgaatcactttcttatccctactgttgtttctcggagtttcgctggctacgtaccctataatagggatacggttgcgaatgtgtactattaagatttattacctgtctactcgcttcgacccacaagggacgtttccgtctgtatcgttcggttgaatgagacgtacttcagaggctaatactactgctatgatctcgaacaataggcgtgagtgtcgggaagtgaagaccagcttgttggacgatacaccaaagctggtaacaatagccctatgtcatgccacgccaaaatatatctctaacaaaaacccctcccataactttcacatctgctgtttccctccaagacgtttcacgaactcaagagttatattgattccgtttcgtccttccaaggcagcccgtcaatccttgggtagtcctgccgagtcttgttcatcaaactatggctaactgaccaatgcaacagcgccatcatatcagctggcgtaggaagccaagtagtgcatacaatatccaccaataacttctgcaggacccctgattccccacactgcggcgttcggcaggtctgttgagtccaggatcgtctgaagatatacctaatgtatgaatctctcttccccggaccgttacaaaagaagactccgcactccgcactccgttttggtccgtacctttttaggatactagcaggaggcaaggatgtgaagtcgggcgacggaccggatggccgtcttgtctttcgtggcatttagtctgctgttacggaagttgcacaatgtatttaaggtagatagaggattctggtttctcttctctctacgcacgattccatcgatatcaactgtcatcatcatgaggtctctcgtccttctgtcgtccgtcctggccctggtggcaccctccaaaggcgccttcacatgtatatctcacacccctcgcttctccagatatctctacctgtgctaaaatcatgcctgtaaatcagggcttggtaccaacgaagccggtgccgaattcggcgagggctcctaccccggcgaactgggcacggaatacatttggcctgacctgggtacgattggtacgctgaggaacgaggggatgaacatattccgcgttgcgttctccatggagcgcctggtgcctgattcgttggctggaccggtagcggacgagtattttcaggacttggtcgaggtgcgcctttcttctctcttgcctcagatcttgggcatggcccaagaacagcaagtcgctaactgcatcagacggtcaacggcattacggccctgggtgcgtatgcagtccttgacccgcataattatggacggtagtatgtatccctagtcaacagaccctacgaacgggccagcaatgctgacgagctaaagctacggcaacattatcacttcaaccgatgactttgcagccttctggaccatccttgccaccgagttcgcttctaatgagcttgtcatcttcgataccagtccgtttattccgacatccactcacttgtgttcttgactgacaaaaatcagataatgaataccacaccatggaccagtcccttgtcctgaacctcaatcaggcggccatagacgctattcgcgcctccggtgccaccagccaatatatcttcgcggaaggaaactcttggactggagcctggacctgggtggatgtcaacgacaacatgaaagcgctcaccgatccccaagataagctcatctacgagatgcaccagtatcttgactccgacggctctgggacgaacacggcctgcgtcagttctactatcggcagcgaacgcgtgacagccgctacaaactggctgcgagagaacgggaaactgggggtgctcggtgagtttgcaggcgcaaacaaccaggtctgcaaggacgcagtcgcagatctgctggagtatcttgaggaaaacagtgatgtatggcttggggccctttggtgggctgctgggccttggtggggtgattatatgttcaacatggagcctacttcgggtattgcataccaggagtactcggaaatcttgcagccgtatttcgtgggaagtcaataatgagtcaaacccaggcatggctaccatggcgacgctcactagtattatgcggacttaaccacaacaatgtcgagtagcgctgctgcgatgagtacggggaccgcagaaagatgaagtcaaatcaaacgaagcggattggctggctgaacatcgtctgcgacagtctgtggagctgcccggtagtgattgagtgttttcacagtgtttgtaaatcttgctagatgtacaagaaggagagctattgatttcttcgacaaggttgaaagtgtatccatagtagaaatgtgtgaaagtggttagtgtagccagtggctcagcccgctgccctggtatgataacacaaattctcttgagtgctgtgtttatccgcgtctaccaacccaagttatccaaagcatctcttatcagcagaagaatcattgcaacccgacttctgtcttcaatccaatctcaacaatgtcactttcgggctcaggctgggacgcgaactagacccaacgactaccctactgaactggagagccgcgcttctaaatgcccggcacatttacctcagccgccgaagagctgagatacgacgacacacttcggggctgatctgcgtagatcagtctcgggaagaactcaacagataatgtctccctggctctgttgccttttttatttttatttttatttttaatttttttttttttttttttttttttggcacaccattcaggaagaatccgcgggcactactaagacatgctaggtcaggcaattaaaacaaagagggtactttcagaggatgagaggttatatggaacgactgaacggaagatcgatgcgggtaagggctgagttgactcctgaaagcgcaagccaaagaatacctcgcggtcaaccacgaaccacccgccgccgccttggtgaggcacccatgaacaaccgcgtgtatttcttctgggctgctacttctgaagagacaaatggccttgaggcacatattgacgagctc

SEQ ID NO: 9 - DNA, 2441 bp, Artificial Sequence
  Synthesized Endo-1,4--glucanase B (eglB) Aspergillus niger
ttttctgtcttgggttacttgtgtgtttatgtgggtgggcttgagggcaagctaatcgatgaattgggacaggtaacgactgtcttgtatgactcgtggtcattcatgacgaagatcctggatgatccgacagcgtatggattccccgatgcgacgtgtatcaatgatgatgggacgtcgtgtatctggtggaataattatcatcccgggatgaagtatcatcttttgcaggctgaggatatgaagccgaagttgaggaagttgggagggtggtagttactttacttagtagtactagtaaatagtatgatgatattgggagaatattatgtgacgtttaggatggcttatatattactagtatgttactaaatttgaatgttgatgcatcaggtatatatatttatgaagtactgccctacgggtattttggctgaattgcatcgtaaatattagcatcgcactgacatgaaagctccacctatgcactaattccgtattcttcttatatgtaaatccttcttgtcccgtcacggacctcatttccgcatcactttcatgaccctgaccctgccaaggcggatcattgaccgagcttagtccctgtcttcgactttatccgccctgtaggcattaatgatacctgcaggtgtgaagatctgcaacgtcttctgtctgaatacctgtttactctctacattcggatgtttcgctttaatggaccgatggatgtagatttctggctaagttacgttacctccgtgctggattaggatagttgggaggaggggaagatataaaggttgcttattgtcttgtcctaaatagcgatagtaaatgtagatttcgttgatcgagcagtcgtagcgatgaagtttcagagcactttgcttcttgccgccgcggctggttccgcgttggctgtgcctcatggctccggacataagaagagggcgtctgtgtttgaatgtaagggctcatttgcctcattgttgctaagctgacagaaaagggttcggatcgaacgagtctggtgctgaatttgggaccaatatcccaggcgtctgggtaagtatccccgccaaagtgtaggcgtatgctgatgtcgcagggaaccgactacatcttccccgacccctcgaccatctctacgttgattggcaagggaatgaacttcttccgcgtccagttcatgatggagaggttgcttcctgactcgatgactggttcatacgacgaggagtatctggccaacttgacgactgtaagatgactcccacttggtgttgacttaatgctgacgagtcaggtggtgaaagcggtcacggatggaggcgcgcatgcgctcatcgaccctcataactatggcagatagtaagtatgcagtaccagagggtgacacttgctaaccaaacagcaacggggagatcatctccagtacatcggatttccagactttctggcagaatctggcgggccagtacaaagataacgacttggtcatgtttgataccagtaagtgcccaccaagccattgaggaccgtgctgactgacaatgcagacaacgaatactacgacatggaccaggatctcgtgctgaatctcaaccaagcagccattaacggcatccgcgctgcaggtgcaagccagtacattttcgtcgaaggcaactcctggaccggagcttggacatgggtcgatgtcaacgataatatgaagaatttgaccgacccagaagacaagatcgtctatgaaatgcaccagtacctagactccgacggttccggcacttcggagacctgtgtctccgggacaatcggaaaggagcggatcactgatgctacacagtggctcaaggacaataagaaggtcggcttcatcggcgaatatgccggggggtccaatgatgtgtgtcggagtgccgtgtccgggatgctagagtacatggcgaacaacaccgacgtatggaagggtgcgtcgtggtgggcagccgggccatggtggggagactacattttcagcctggagcccccagatggaactgcttacacgggtatgctggatatcctggagacgtatctctgagaactgggtggggtcgcagatgcggtgcgtcggagaactatacggagtttcttatcagagtggacggtggtggtacagagaggcgtactagaatgaattagtggcagcgcactgactgacgtcacaagacattgctttttttgtgaagctccatctttgtgcattctgttgagtgctacctgtaggcagtttgtcgctgaaatagttgcacgttgtactccgtaacggcacttggtggattcttatcaacgatcatcgagtatagcccaacgaattgcgtgggatgtagagatctgacgacccactggtcgtaggatatacttagctctcctcatcagccttatcggataaatca

SEQ ID NO: 10 - DNA, 3959 bp, Artificial Sequence
  Synthesized Endo-1,4--glucanase C (eglC) Aspergillus niger
atcgataaggtccatgtcatgagagggaaggaagttcataatttkgggagggcttatattagttccccaccaccccctcattagggtgtcaaagggccaatctttgaacaatagcctcgatcgagtccagtccgaccgggcggcaaaagcgtcaagtgaaactcctatctgaaagtgtgcagtttttcggcttgttgtggcaggtcttgcaggttatgatctcggtatgactagtcagtaggccagacgagtcatagctgccaagcnnnntttcaccgagacatcgtcaagaggtactcctttgggcagggcaaaccttgcagcagccaatgtgaaatttccggctggaaacggtgctttcgggataaagggcacgatggctgcgctagccctgcaggtgtcaggagaaattcccgaccgtgcctgtccggaactgcgtaccatcagaatggcgtgcattcatccccatctgcagctcgttgttccgccaatcacaacagcgtatctgtccgtcaccgccctatgaatttcaagcagtattcagcttcgatgaagaaaatggcatcctgatccctctcaagaatgctcgaaagcaaggggatatgcttcaagtctaagctcctctgtcaatatgcaatgactggctaattcggaggctgctccgtgataaatcagtcacgcagcaacatctgaccccatctcattagaattaatctattcggctattcggtccttttcccgggtatatttcttcgaccctggttctgcagaatcatgttgttcattgcggagcaggtcaagattcgtctgaccacacaatgccttggtccgttccaggtgaggtatagcatcttcgttcatgtcaactaatgcagggccatctgcatgtcacacatgaacgcacctcgtcacccgtaggattgtgcctgaagggcagcttcaatgcttgaacctgctggatatttaagctccatgttctcccacatggattgacaagttgtcagtagcaccaactcggtcaacattcactcgtcaccatgcggcgtacattcgcagccctcgtagcgggctacctgctggactcggtccacgctgccgcatcgcaggcatatacctggaagaatgtggtcacagggggtatgacatatcttgcaatatgagggattgacaagctaattgatcaggcggcggaggcttcacgccgggtatcgtcttcaacccttcagctaaaggtgtcgcatatgctcgcaccgacatcggcggtgcatatcggttgaactcggacgatacatggaccccgttgatggactgggccaacaactctaactggtacgtaagcttctcttacgagatgcctaggctcactgtccaggcacgattggggcattgatgcgatcgcgaccgaccccgtcgataccgatcgggtatatgttgcagttggcatgtacaccaacgactgggatcccaatgacggctccatcctgcggtcgaccgaccagggcgatacctgggaagagacaaagcttcccttcaaagttggagggaatatgcccggtcggggtgttggcgaggtaagtcctatatctgggttttgctagcatacactgaccgctgcagcgtctggctgtggatccgaatgacaacagcatcttgtactttggtgcccggagtggcaatggcttgtggaaaagcacggactacggcgagacatggtccaatgtgactgccttcaagtggaccggcacttatttccaagattccagctcgacctacacgtctgatccggtgggaattgcctgggtcaccttcgactcgacctcagggtccagtggctctccgactccgcgcatctttgtcggtatgtgaaaaattctagatatctcaggacagcccactgaccattacaggtgtggtggacactggagaatccgtctttgtgtccgaagatgctggcgaaacttgtaagcttctccacgatcgcaattaaacccatctaaccaacttagggacttgggtatctggagagcccatgtacggcttcctcccccataaaggcatcctgtcgccttctgagcacacactctacatttcctactccaacggcgccggtccatacgatggcaccaatggcaccgtccacaagtacaacatcaccagcggcgtctggaccgacatcagtcccacctccatgacagacacctactatggctatggcggactggcagtcgatctccaagtgcccggaactgtcatggtagcagcactcaactgctggtggcctgatgagctgatctggcgcagcacggattctgggggcacctggtcacccatctgggcttggaacggctatcccagcatcaactactactactcttatgacatctccaacgccccatggctacaagacgacacatctacagacgaatttcccgtccgcgtcggctggatggtcgaagccctcgccattgatcccttcgactctgaccactggctttacggcactggcgagaccatctacggcggccatgacctccaaaattgggacagcgaacacaacgtaactatcgaatctcttgccgtaggcatcgaagaaatggctgttctgggtctcatcactcccccaggcgggcctgcccttctctctgcggtcggcgacgatggcggcttctaccacacttccctgaccaccgccccatcacaatactaccacaccccgacttacagttcaacaaacggcatcgactacgccggcaacaagcccgctaacatcgtccgctctggatccagcgattccgaccctactctcgctttatcctccagtttcggcgaatcttggtacgccgactacgctgcttcttcctctactgcgaccggccaagtcgccctctcagccgatgcagacaccatcctcctcatgaacagcgacggtgcctatcgttccgcaaactcggctactcttagcgcagtctcctctctgccctctggagctgtcatcgcctccgacaaagctaacaacacctacttctacggagcctccggctcttccttctatctttcatccgacaccgccgcgaccttcactgtcaccaccaccctcggcagtagcacaaccgccaacgccatccgcgcgcaaccctctctcgcaggcgatgtctgggtcagtacagacacaggtctcttccactcaaccaactacggcaaatccttcacccagattgggagcggatgcacagaggggtggagtttcggattcggaaagccctcttcggacggcgactacccggtcctatttggattctttaccgtcgatggtgttaccggtctcttcaaaaccgaagatcagggtgttaattggcagataatttccgacgccgagcacgggttcgggtctgcttcggcaaatgtagtcaatggggatcttcagaactatggcagggtgtttgttgggacaaatgggcgaggaatcttctatggtgatccgagtggaactctgccgagtgcgactgctactgcgtctagtgcttcgtcgacagcggtgaagagttcgacctcgacttcgacatccaaggtaggttccagcacaactgtttcttcgagtacggctacaactatcactacgagcagtattaagtcgaccactttgacgacgactacaaagtcgagtagttcgactacttcgacgtcatccactgctactgggacggcctcggcatatgggcagtgcggtgggagcgggtttacgggaccgacgcagtgcccgagtgggtggacgtgcacgtatgagaatgagtattattcgcagtgtaagtctatccctggtatagcgacggacaggggctgatatatgatgcaggtctggagggatgagtgagagtagggcggtttgagttgtgttgatgttggggtatattgaattcgactgtttgatttgtagtacttgataacaaatatttatcgat

SEQ ID NO: 11 - DNA, 1074 bp, Artificial Sequence
  Synthesized Endo-b(1,4)-glucanase AN1602.2 Aspergillus nidulans
atgaagttctccagcgtcctcgctctggcagcgagcgccaaactcgtcgcttcccacgccaccgtcttcgctgtctggatcaatgacgaagaccagggcctcggcaacaccgctgatggctacattcgcacaccccccaacaacagccccgtcactgacgtgacctctactgacctcacctgtaacgtcaacggcgaccaggccgctgcgaagactctcgaagtcgccgctggtgacaagatcacctttgaatggcaccacaactcccgcgattcttccgacgacatcatcgccgactcccacaagggccctgtcctcgtctacatggcccccacggaggccggctctgcgggcaagaactgggtcaagatctacgaggacggatacaacgatggtacatgggctgtcgacactctcatcgcaaacaagggcaagcactcggtgaccgtccccgacgtgccggccggtaactacctcttccgccctgagatcattgccctgcatgagggcaaccgcgagggaggcgcgcagctctacatggagtgcgtccagttcaaggtcacctctgacggtaccactcagctgccggagggtgtcagtctgccgggtgcctacactgccaccgacgagggaattctcttcgacatctacagctcctttgactcctaccccatccctggcccggccgtttgggacggcgcttcgtctggctctggctcttctggctctggctccagctccagtgctgccgctacatccagcgctgagaagactgctacctccaccactgccgctgccacaaccaccgctgttgcgacgtccacctcgtctgccactcaggtccaacccacctccgtggcgaccttcaccaccagcgtccgacccaccacctccgctgcccccactacgtctgccccgacttcctctgccgcccctaccggcggcaccggcactggctccatccaaatctaccagcagtgcggtggaatgaactacaagggtgccaccggctgcgcctctggcctgacctgcaagcagtggaacccttactaccaccagtgtgtccaggcttag

SEQ ID NO: 12 - DNA, 1005 bp, Artificial Sequence
  Synthesized Endo-b(1,4)-glucanase AN5214.2 Aspergillus nidulans
atgaaggtcaacactcttttggttgccgtagcggctggtaccgcgatggctgcaccccagctcaagaagcgcgccggttttacgttttttggagtcaccgaggctggcgctgagttcggcgagaaaagtattcctggtgtttggggcactgactacaccttccccgatactgaatccatcttgaccctcatctcgaagggtttcaacactttccgcattcccttccttatggagcgtctgacgcctgagatgacgggctctttcgacgaaggatacctgaaaaatctgacttcggtcgtcaatgcagtcaccgatgcaggagcatgggccatcgttgacgcccaaaactttggccgattcaacggcgagatcataagcagcgcctccgacttccagacttggtggaagaacgtagcggccgagttcgcggacaataagaacgtcattttcgataccactaacacgtctggggcagacaacgaattccacgacatggaccagaccctcgtgctggatctcaaccaagccgccatcaacggcatccgcgcagccggcgccacctcgcaatacatcttcgtcgagggtaactcgtacaccggcgcctggacatggacggacaacaatgacaacctgaaatccctcaccgacccgcaggacaagatcgtctacgaaatgcaccagtacctcgatactgacggctccggaacacacgagacctgtgtttcggagacaatcggcgccgagagggttgaatccgcgacgcagtggctgaaagacaacggcaaactcggtgtcattggtgaatttgcgggcggcaataacgagatatgccgcgcagcggtcaagagtctactggatgcgctgaaggagaacgatgatgtttggctcggtgcgctttggtgggctgctgggccttggtgggaagactatatgttcagcatggagccgacggatggcattgcgtatacggggatgctcagtacgttggaggcgtacatgaattaa

SEQ ID NO: 13 - DNA, 981 bp, Artificial Sequence
  Synthesized Endo-b(1,4)-glucanase AN1285.2 Aspergillus nidulans
atgaggtctctcgtccttctgtcgtccgtcctggccctggtggcaccctccaaaggcgccttcacatggcttggtaccaacgaagccggtgccgaattcggcgagggctcctaccccggcgaactgggcacggaatacatttggcctgacctgggtacgattggtacgctgaggaacgaggggatgaacatattccgcgttgcgttctccatggagcgcctggtgcctgattcgttggctggaccggtagcggacgagtattttcaggacttggtcgagacggtcaacggcattacggccctgggtgcgtatgcagtccttgacccgcataattatggacggtactacggcaacattatcacttcaaccgatgactttgcagccttctggaccatccttgccaccgagttcgcttctaatgagcttgtcatcttcgataccaataatgaataccacaccatggaccagtcccttgtcctgaacctcaatcaggcggccatagacgctattcgcgcctccggtgccaccagccaatatatcttcgcggaaggaaactcttggactggagcctggacctgggtggatgtcaacgacaacatgaaagcgctcaccgatccccaagataagctcatctacgagatgcaccagtatcttgactccgacggctctgggacgaacacggcctgcgtcagttctactatcggcagcgaacgcgtgacagccgctacaaactggctgcgagagaacgggaaactgggggtgctcggtgagtttgcaggcgcaaacaaccaggtctgcaaggacgcagtcgcagatctgctggagtatcttgaggaaaacagtgatgtatggcttggggccctttggtgggctgctgggccttggtggggtgattatatgttcaacatggagcctacttcgggtattgcataccaggagtactcggaaatcttgcagccgtatttcgtgggaagtcaataa

SEQ ID NO: 14 - DNA, 1293 bp, Artificial Sequence
  Synthesized Endo-b(1,4)-glucanase AN3418.2 Aspergillus nidulans
atggctctgttactatctctcagccttcttgccacaacaatctcagcccaacagattgggacgccagaaatccggccacgtcttactacctaccactgtacttccgccaacggctgtacagagcagaatacctctgtggtgctcgatgccgccacgcaccccatccacgatgcatccaaccccagtgtctcctgcaccacctcaaatgggctaaaccctgctctatgcccagacaagcagacttgcgcagacaactgcgtcatcgacggcataactgactatgctgcgcacggagtcgaaacccatgggtcgcggttgacactcactcaataccgaaacgtgaacggggcgctctcctctgtttcaccgagggtctatctcgttgatgagtccgaccctgatgagcaggagtatcgagccttgtccctgctcgcccaagaatttaccttcactgtcaacgtctccgcgctcccatgcgggatgaacggcgcgctatatctctccgaaatgtctccctccggcgggcgcagcgcgctcaaccccgccggagcctcctatggcacaggctactgcgatgcccaatgttatgtgaatccctggatcaacggcgagggaaacatcaacggctacggagcctgctgcaacgagatggacatctgggaggctaattcgcggagtacggggttcacgcctcatgcttgtttatatgagccggaggaaactgagggaagaggggtatacgaatgcgcctccgaagatgagtgcgatagcgcgggcgagaatgacggcatctgcgataagtggggatgcggctttaacccgtatgctctgggaaatacagagtactacggccgtggccaagggtttgaagtcgacactaaagagcccttcacggtcgtgacacagttcctgacggatgacggaacaagtacaggtgctttaaccgagattagacggctatatatccaaaacgggcaggtcatcgagaacgcagttgtctcgtctggtgcagactcgctgaccgattccctctgcgcctctaccgcgtcatggttcgactcatacggaggaatggaagggatgggaagggcgcttggccgtgggatggtcctcgccatgagtatctggaatgatgcgggtggctacatgcagtggctcgacggtggggatgcaggaccctgtaatgccaccgagggcgcaccggaatttattgaggagcatacgccgtggacaagggttgtctttgaagatttgaagtggggtgatattggcagtactttccaggcgtcttag

SEQ ID NO: 15 - DNA, 2869 bp, Artificial Sequence
  Synthesized Avicelase (AviIII) Acidothermus cellolyticus
atggatcgttcggagaacatccgtctgactatgagatcacgacgattggtatcactgctcgccgccactgcgtcgttcgccgtggccgccgctctgggagttctgcccatcgcgataacggcttctcctgcgcacgcggcgacgactcagccgtacacctggagcaacgtggcgatcgggggcggcggctttgtcgacgggatcgtcttcaatgaaggtgcaccgggaattctgtacgtgcggacggacatcggggggatgtatcgatgggatgccgccaacgggcggtggatccctcttctggattgggtgggatggaacaattgggggtacaacggcgtcgtcagcattgcggcagacccgatcaatactaacaaggtatgggccgccgtcggaatgtacaccaacagctgggacccaaacgacggagcgattctccgctcgtctgatcagggcgcaacgtggcaaataacgcccctgccgttcaagcttggcggcaacatgcccgggcgtggaatgggcgagcggcttgcggtggatccaaacaatgacaacattctgtatttcggcgccccgagcggcaaagggctctggagaagcacagattccggcgcgacctggtcccagatgacgaactttccggacgtaggcacgtacattgcaaatcccactgacacgaccggctatcagagcgatattcaaggcgtcgtctgggtcgctttcgacaagtcttcgtcatcgctcgggcaagcgagtaagaccatttttgtgggcgtggcggatcccaataatccggtcttctggagcagagacggcggcgcgacgtggcaggcggtgccgggtgcgccgaccggcttcatcccgcacaagggcgtctttgacccggtcaaccacgtgctctatattgccaccagcaatacgggtggtccgtatgacgggagctccggcgacgtctggaaattctcggtgacctccgggacatggacgcgaatcagcccggtaccttcgacggacacggccaacgactactttggttacagcggcctcactatcgaccgccagcacccgaacacgataatggtggcaacccagatatcgtggtggccggacaccataatctttcggagcaccgacggcggtgcgacgtggacgcggatctgggattggacgagttatcccaatcgaagcttgcgatatgtgcttgacatttcggcggagccttggctgaccttcggcgtacagccgaatcctcccgtacccagtccgaagctcggctggatggatgaagcgatggcaatcgatccgttcaactctgatcggatgctctacggaacaggcgcgacgttgtacgcaacaaatgatctcacgaagtgggactccggcggccagattcatatcgcgccgatggtcaaaggattggaggagacggcggtaaacgatctcatcagcccgccgtctggcgccccgctcatcagcgctctcggagacctcggcggcttcacccacgccgacgttactgccgtgccatcgacgatcttcacgtcaccggtgttcacgaccggcaccagcgtcgactatgcggaattgaatccgtcgatcatcgttcgcgctggaagtttcgatccatcgagccaaccgaacgacaggcacgtcgcgttctcgacagacggcggcaagaactggttccaaggcagcgaacctggcggggtgacgacgggcggcaccgtcgccgcatcggccgacggctctcgtttcgtctgggctcccggcgatcccggtcagcctgtggtgtacgcagtcggatttggcaactcctgggctgcttcgcaaggtgttcccgccaatgcccagatccgctcagaccgggtgaatccaaagactttctatgccctatccaatggaaccttctatcgaagcacggacggcggcgtgacattccaaccggtcgcggccggtcttccgagcagcggtgccgtcggtgtcatgttccacgcggtgcctggaaaagaaggcgatctgtggctcgctgcatcgagcgggctttaccactcaaccaatggcggcagcagttggtctgcaatcaccggcgtatcctccgcggtgaacgtgggatttggtaagtctgcgcccgggtcgtcatacccagccgtctttgtcgtcggcacgatcggaggcgttacgggggcgtaccgctccgacgactgtgggacgacctgggtactgatcaatgatgaccagcaccaatacggaaattggggacaagcaatcaccggtgaccacgcgaatttacggcgggtgtacataggcacgaacggccgtggaattgtatacggggacattggtggtgcgccgtccggatcgccgtctccgtcggtgagtccgtcggcttcgccgagcctgagcccgagcccgagcccgagcagctcgccatcgccgtcgccgtcgccgagctcgagtccatcctcgtcgccgtctccgtcgccgtcaccatcgccgagtccgtctcggtctccgtcaccatcggcgtcgccgagcccgtcttcgtcaccgagcccgtcttcgtcaccgtcttcgtcgccgagcccaacgccgtcgtcgtcgccggtgtcgggtggggtgaaggtgcagtataagaataatgattcggcgccgggtgataatcagatcaagccgggtttgcaggtggtgaataccgggtcgtcgtcggtggatttgtcgacggtgacggtgcggtactggttcacccgggatggtggctcgtcgacactggtgtacaactgtgactgggcggcgatcgggtgtgggaatatccgcgcctcgttcggctcggtgaacccggcgacgccgacggcggacacctacctgcagn

SEQ ID NO: 16 - DNA, 1746 bp, Artificial Sequence
  Synthesized CellobiohydrolaseI (CBHI) Trichoderma reesii
tttgaggcacagaaacccaatagtcaaccgcggactgcgcatcatgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactgggacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgggctcaacgatgattactgcacagctgaggaggcagaattcggcggatcctctttctcagacaagggcggcctgactcagttcaagaaggctacctctggcggcatggttctggtcatgagtctgtgggatgattactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcgaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgtaaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggc

SEQ ID NO: 17 - DNA, 1849 bp, Artificial Sequence
  Synthesized CellobiohydrolaseI (CBHI) (Gux1B) Neurospora crassa
gagtctgtaaccaaactctttacccgtccttgggtccctgtagcagtatatccattgtttcttatataaaggttagggggtaaatcccggcgctcatgacttcgccttcttcccttatctgatcgggcaccggaaaccaattgcactcaaaatgagggcctcgctcctggccttctccctcgctgccgccgtggccggcggccagcaggccggcactctcaccgccaagaggcacccatccctcacatggcagaagtgcaccaggggggggtgcccgaccctgaacaccacgatggtgctcgacgcgaactggcgctggactcacgccacgtccggctccacgaagtgctacacgggcaacaagtggcaggcgacgctctgccccgatggcaagtcgtgcgcggcgaactgcgcgctggacggcgccgactacaccggcacctacgggatcaccgggagcggctggtccctcacgctccagttcgtcacggacaacgtcggcgcccgtgcctacctgatggcggacgacacgcagtaccagatgttggagctcctgaaccaggagttgtggttcgacgtcgatatgtcgaacatcccgtgcggtctgaacggcgccctctacctctcggcgatggacgcggatgggggcatgaggaagtacccgaccaacaaggctggcgctaagtacgctaccggttactgcgacgctcagtgcccccgtgatctcaagtacatcaacggtatcgccaacgttgagggctggaccccttccaccaacgatgctaacggtattggtgaccacggatcttgctgctctgagatggatatctgggtttgtttgccgattttcctttcatcattagcatcacaggtaactaacacccacctaaggaagcgaacaaagtctctacagcgttcaccccgcacccctgcaccaccatcgaacagcacatgtgcgagggtgactcctgcggtggtacctattccgacgaccgctatggcgtactttgcgatgccgatggttgtgacttcaacagctaccgcatgggcaacaccaccttctacggtgagggcaagactgtcgataccagctccaagttcaccgttgtcacccagttcatcaaggactccgctggcgatcttgctgagatcaaggccttctacgtccagaacggaaaagtcattgagaactctcagtccaacgttgatggagtttctggcaactccatcacccagtctttctgcaagtctcagaagactgctttcggcgatatcgatgacttcaacaagaagggtggcctgaagcaaatgggcaaggcccttgcccaagccatggtcctcgtcatgtccatctgggacgaccatgccgccaacatgctctggctcgactccacctaccctgtcccgaaggtccccggtgcttaccgtggcagtggccctaccacctcgggtgtcccagctgaggtcgacgccaatgctcccaactccaaggtcgccttctccaacatcaagttcggccacctcgggatctctccttttagcggcggctcttccggcacccctccttccaacccttcgagctccgcaagcccgacttcctccactgctaagccttcttccacctctactgcctccaaccccagcggtaccggtgctgctcactgggctcagtgcggtggtattggcttctctggccccaccacttgcccagagccctacacttgcgcaaaagatcacgacatttactcccagtgcgtgtaaattactagcctgctagggtaacctttttggttcctctactacggcagctaggtgaactcgactgcgaagcaaaaaggaacttcgagaa

SEQ ID NO: 18 - DNA, 2193 bp, Artificial Sequence
  Synthesized CellobiohydrolaseII (CBHII) Trichoderma reesii
tcgaactgacaagttgttatattgcctgtgtaccaagcgcgaatgtggacaggattaatgccagagttcattagcctcaagtagagcctatttcctcgccggaaagtcatctctcttattgcatttctgcccttcccactaactcagggtgcagcgcaacactacacgcaacatatacactttattagccgtgcaacaaggctattctacgaaaaatgctacactccacatgttaaaggcgcattcaaccagcttctttattgggtaatatacagccaggcggggatgaagctcattagccgccactcaaggctatacaatgttgccaactctccgggctttatcctgtgctcccgaataccacatcgtgatgatgcttcagcgcacggaagtcacagacaccgcctgtataaaagggggactgtgaccctgtatgaggcgcaacatggtctcacagcagctcacctgaagaggcttgtaagatcaccctctgtgtattgcaccatgattgtcggcattctcaccacgctggctacgctggccacactcgcagctagtgtgcctctagaggagcggcaagcttgctcaagcgtctggtaattatgtgaaccctctcaagagacccaaatactgagatatgtcaaggggccaatgtggtggccagaattggtcgggtccgacttgctgtgcttccggaagcacatgcgtctactccaacgactattactcccagtgtcttcccggcgctgcaagctcaagctcgtccacgcgcgccgcgtcgacgacttctcgagtatcccccacaacatcccggtcgagctccgcgacgcctccacctggttctactactaccagagtacctccagtcggatcgggaaccgctacgtattcaggcaacccttttgttggggtcactccttgggccaatgcatattacgcctctgaagttagcagcctcgctattcctagcttgactggagccatggccactgctgcagcagctgtcgcaaaggttccctcttttatgtggctgtaggtcctcccggaaccaaggcaatctgttactgaaggctcatcattcactgcagagatactcttgacaagacccctctcatggagcaaaccttggccgacatccgcaccgccaacaagaatggcggtaactatgccggacagtttgtggtgtatgacttgccggatcgcgattgcgctgcccttgcctcgaatggcgaatactctattgccgatggtggcgtcgccaaatataagaactatatcgacaccattcgtcaaattgtcgtggaatattccgatatccggaccctcctggttattggtgagtttaaacacctgcctccccccccccttcccttcctttcccgccggcatcttgtcgttgtgctaactattgttccctcttccagagcctgactctcttgccaacctggtgaccaacctcggtactccaaagtgtgccaatgctcagtcagcctaccttgagtgcatcaactacgccgtcacacagctgaaccttccaaatgttgcgatgtatttggacgctggccatgcaggatggcttggctggccggcaaaccaagacccggccgctcagctatttgcaaatgtttacaagaatgcatcgtctccgagagctcttcgcggattggcaaccaatgtcgccaactacaacgggtggaacattaccagccccccatcgtacacgcaaggcaacgctgtctacaacgagaagctgtacatccacgctattggacctcttcttgccaatcacggctggtccaacgccttcttcatcactgatcaaggtcgatcgggaaagcagcctaccggacagcaacagtggggagactggtgcaatgtgatcggcaccggatttggtattcgcccatccgcaaacactggggactcgttgctggattcgtttgtctgggtcaagccaggcggcgagtgtgacggcaccagcgacagcagtgcgccacgatttgactcccactgtgcgctcccagatgccttgcaaccggcgcctcaagctggtgcttggttccaagcctactttgtgcagcttctcacaaacgcaaacccatcgttcctgtaaggctttcgtgaccgggcttcaaacaatgatgtgcgatggtgtggttcccggttggcggagtctttgtctactttggttgt

SEQ ID NO: 19 - DNA, 3687 bp, Artificial Sequence
  Synthesized GuxA Acidothermus cellolyticus
atggagcgaacccaacaatccggacggaactgcaggtaccagagaggaacgacacgaatgcccgccatctcaaaacggctgcgagccggcgtcctcgccggggcggtgagcatcgcagcctccatcgtgccgctggcgatgcagcatcctgccatcgccgcgacgcacgtcgacaatccctatgcgggagcgaccttcttcgtcaacccgtactgggcgcaagaagtacagagcgaagcggcgaaccagaccaatgccactctcgcagcgaaaatgcgcgtcgtttccacatattcgacggccgtctggatggaccgcatcgctgcgatcaacggcgtcaacggcggacccggcttgacgacatatctggacgccgccctctcccagcagcagggaaccacccctgaagtcattgagattgtcatctacgatctgccgggacgcgactgcgcggcgctcgcctccaacggcgaactgcccgctacggcagcaggtttgcagacctatgaaacgcagtacatcgatccgattgcgagtatcctgagcaatccgaagtactccagcctgcggatcgtgacgatcattgagccggactcgctgccaaacgcggtcaccaatatgagcattcaagcgtgtgcaacggcggtgccgtattacgagcaaggcatcgagtacgcgctcacgaaattgcacgccattccgaacgtgtacatctacatggacgccgcccactccggctggcttgggtggcccaataatgccagcggatacgtacaggaagtccagaaggtcctcaacgcgagcatcggggtcaacggcatcgacggcttcgtcaccaacacggcgaattacacgccgttgaaggagccgttcatgaccgccacccagcaggtcggcggacagccggtggagtcggcgaatttctaccagtggaatcctgacatcgacgaagccgactacgcggttgacttgtactcgcggctcgtcgccgctggctttccaagcagcatcggcatgctcatcgacaccttacgcaacggttggggtggtccgaacgaaccaacaggcccgagcaccgcgaccgatgtcaacaccttcgtcaaccagtcgaagattgaccttcggcagcaccgcggcctgtggtgcaaccagaacggtgcgggcctcggccagccgccgcaggcaagcccgacggacttcccgaacgcgcacctcgacgcgtatgtctggatcaagccgccgggtgagtcggacggcacaagcgctgcgagcgatccgacaactggcaagaagtcggaccccatgtgcgacccgacgtacacgacgtcgtacggggtactgaccaacgcgttaccgaactccccgatcgccggccagtggttcccggcgcagtttgaccagcttgtcgcgaacgcacggccagcggtgccgacgtcgaccagctcgagcccgccgcctccgccgccgagtccgtcggcttcgccgagtccgagcccgagtccgagcccgagcagctcgccatcgccgtcgccgtctccgagctcgagcccgtctccgtcgccgagcccgagtccgagcccgagtagctcgccgtcgccgtctccgagctcgagcccgtctccgtcgccgagcccgagtccgagcccgagtagctcgccgtcgccgtctccgagctcgagcccgtctccgtcgccgagcccgagtccgagcccgagtagctcgccgtcgccgtctccgacgtcgtcgccggtgtcgggtgggctgaaggtgcagtacaagaacaatgattcggcgccgggtgataaccagatcaaaccgggtctccagttggtgaataccgggtcgtcgtcggtggatttgtcgacggtgacggtgcggtactggttcacccgggatggtgggtcgtcgacactggtgtacaactgtgactgggcggcgatggggtgtgggaatatccgcgcctcgttcggctcggtgaacccggcgacgccgacggcggacacctacctgcagttgtcgttcactggtggaacgttggccgctggtgggtcgacgggtgagattcaaaaccgggtgaataagagtgactggtcgaatttcaccgagaccaatgactactcgtatgggacgaacaccaccttccaggactggacgaaggtgacggtgtacgtcaacggcgtgttggtgtgggggactgaaccgtccggcaccagccccagccccacaccatccccgagcccgagcccgagcccgagcccgggtggggatgtgacgccgccgagtgtgccgaccggcttggtggtgacgggggtgagtgggtcgtcggtgtcgttggcgtggaatgcgtcgacggataacgtgggggtggcgcattacaacgtgtaccgcaacggggtgttggtgggccagccgacggtgacctcgttcaccgacacgggtttggccgcgggaaccgcgtacacctacacggtggccgcggtggacgctgcgggtaacacctccgccccatccacccccgtcaccgccaccaccacgagtcccagccccagccccacgccgacggggaccacggtcaccgactgcacgcccggtcctaaccagaatggtgtgaccagcgtgcagggcgacgaataccgggtgcagaccaatgagtggaattcgtcggcccagcagtgcctcaccatcaataccgcgaccggtgcctggacggtgagcactgcgaacttcagcggtgggaccggcggtgcgcccgcgacgtatccgtcgatctacaagggctgccactggggcaactgcaccacgaagaacgtcgggatgccgattcagatcagtcagattggttcggctgtgacgtcgtggagtacgacgcaggtgtcgtcgggcgcgtatgacgtggcctacgacatttggacgaacagtaccccaacgacaaccggtcagccaaacggtaccgaaatcatgatttggctgaattcgcgtggtggggtgcagccgttcgggtcgcagacagcgacgggtgtgacggtcgctggtcacacgtggaatgtctggcagggtcagcagacctcgtggaagattatttcctacgtcctgacccccggtgcgacgtcgatcagtaatctggatttgaaggcgattttcgcggacgccgcggcacgcgggtcgctcaacacctccgattacctgctcgacgttgaggccgggtttgagatctggcaaggtggtcagggcctgggcagcaactcgttcagcgtctccgtgacgagcggcacgtccagcccgacaccgagcccgagcccgacgccgacaccgagcccgacgccgacaccgtctccgagcccgaccccgtcgccgagtccgaccagctcgccgtcgtcgtcgggtgtggcgtgccgggcgacgtatgtggtgaatagtgattggggttctgggtttacggcgacggtgacggtgacgaataccgggagccgggcgacgaacgggtggacggtggcgtggtcgtttggtgggaatcagacggtcacgaactactggaacactgcgttgacccaatcaggtgcatcggtgacggcgacgaacctgagttacaacaacgtgatccaaccgggtcagtcgaccaccttcggattcaacggaagttactcaggaacaaacgccgcgccgacgctcagctgcacagccagctga

SEQ ID NO: 20 - DNA, 1581 bp, Artificial Sequence
  Synthesized Cellobiohydrolase AN0494.2 Aspergillus nidulans
atggcatcttcattccagttgtacaaagctctcctctttttctcttctctgctttctgcagtgcaagcccagaaggtcggcactcagcaagctgaagtgcaccccggcctgacctggcagacctgcacgagctccggcagctgcaccaccgtcaacggcgaggtcactattgacgcaaactggcgctggctgcacaccgtcaatgggtacaccaactgctatactggcaacgaatgggatacctccatctgcaccagcaacgaggtttgcgcggaacaatgcgctgtcgacggtgctaactatgcctccacatacggcatcaccacatccggcagctcgctgcgtctgaacttcgtcacgcagtcgcagcagaagaatatcggttccagagtctatctcatggatgacgaggatacatacaccatgttctacctgctcaacaaggaattcacttttgacgtcgacgtctccgagctcccctgcggtctcaacggggcggtctactttgtgtctatggacgccgacggcggcaaatcccgctatgccaccaacgaagccggtgccaaatacggcacgggatactgcgactctcagtgcccgcgggacctcaagttcatcaacggcgtcgccaacgtcgagggctgggaatcctccgatacgaaccccaacggcggcgtcggcaatcacggctcctgctgcgcagagatggatatctgggaggcaaacagcatttccactgctttcactccccatccctgcgataccccgggccagaccctctgcaccggtgactcatgcggtgggacctatagcaacgaccgctacggcggcacctgcgaccccgatggctgcgactttaactcctaccgtcaggggaacaagaccttctacgggccaggcctgacagtcgacacgaacagcccggtcacagtggtgacccagttcctgacagacgacaacacggacacaggcaccctctcggaaatcaaacgcttctatgtccagaacggcgtcgtcatccccaactccgagtcgacctaccccgctaatccgggtaactcgatcacaacggagttctgcgagtcgcaaaaggaactcttcggcgacgtcgatgttttctccgcccacggcggcatggcgggcatgggcgccgcgttggaacaaggcatggtccttgtactgtccctgtgggacgacaactactcaaacatgctctggctcgactcgaattaccccacggacgcggacccgactcaaccaggtatcgcgcgcgggacgtgcccgacggactcgggcgttccgtctgaagtcgaggcccaatatccgaatgcgtatgtcgtgtactcgaacatcaagtttggtcctattggaagtacctttggcaacggtggaggctcagggccaacaacaacggtgacgacgagtaccgctactagtacaactagctcggcgacgtcgacggctaccggtcaggcgcagcactgggagcagtgtggtgggaatggctggactggtccgacggtctgcgctagcccctgggcttgcacagtggtgaactcatggtactcgcagtgtctgtaa

SEQ ID NO: 21 - DNA, 1353 bp, Artificial Sequence
  Synthesized Cellobiohydrolase AN5282.2 Aspergillus nidulans
atgcactactctgcttcaggtctcgcgttggcattcttgcttcctgcaattcaggctcagcaaactctctatgggcaatgtgggggatctggctggaccggtgctacgagctgtgtagctggcgcagcgtgttctactctgaaccaatggtacgctcagtgcttgccagccgcaaccaccacgtctactaccctgaccacgacaacatcttcagtaaccacgacgagcaatcctggctctacgacaactacaagctctgttactgtcactgccactgcatctggaaaccctttcagtggctaccagctctacgtcaacccttactactcatctgaggtgcaaagtatcgccattccatccctcactgggacactctcctcgcttgcaccggcagccactgctgccgccaagacaagagatgttgccgcaaaggtgcccacaatggcgacctacctcgcggatatccggtcacagaatgctgccggagccaaccctcctattgcaggccaatttgttgtttatgaccttccggaccgtgactgcgccgctctggccagtaatggcgagtttgccatttcagatggcggggtgcagcattataaggattatattgattccatccgcgaaattctcgtggagtactcagacgtccacgtaatcctggttattgaaccggacagtttagctaatcttgtcacaaacttgaacgttgctaaatgtgccaacgcccagagcgcttacctggaatgcacaaactacgcggtcacgcagctcaaccttccgaacgtggccatgtatcttgacgctggacatgccggctggcttggctggccagcaaacctccagcccgccgcaaacctctacgcaggagtctactcagacgccgggtcaccggccgcacttcgtggtcttgctaccaacgtcgccaattacaacgcctgggcaatcgatacctgcccttcatacacgcaaggtaactccgtttgtgacgaaaaggactacatcaacgccctcgctcccctgcttcgcgctcagggctttgacgctcacttcatcacagatacgggccgcaacgggaagcagccaacaggccaacaagcctggggcgactggtgcaacgtcatcggcacgggcttcggcgcgcgcccatccacgaacacaggtgattccttattggatgcttttgtctgggttaaacctggtggcgagagtgacgggacttctgatacgtccgcggcgcggtatgatgcgcattgcgggtatagcgacgcgctgcagccagcgcccgaggcgggaacttggtttcaggcatatttcgtgcagcttttacagaatgcgaacccgtctttctag

SEQ ID NO: 22 - DNA, 1341 bp, Artificial Sequence
  Synthesized Cellobiohydrolase AN5176.2 Aspergillus nidulans
atgtatcaacgcgctcttctcttctctgctcttttatccgtgtcgcgggcccagcaggcaggcaccgcacaggaggaagtgcatccctctttgacatggcagaggtgcgaggccagtggatcgtgcaccgaagttgcgggttccgtcgtgctggattccaactggcgctggacacactcggttgatgggtacaccaactgctacactggcaacgagtgggatgcaaccttgtgccccgacaacgagtcatgcgctcagaactgcgctgttgacggcgccgactacgaggctacctacggtatcacgtcaaatggcgactcgttgactcttaagttcgtcactggttccaacgtcggctctcgtgtctacctgatggaggacgacgagacctaccagatgttcgacctgctcaacaacgagttcacctttgacgttgacgtctccaacctcccttgcggtctgaacggcgctctctacttcacctcgatggacgcagatggcggcttgagcaagtacgaaggcaacaccgccggcgccaagtatggaactggctactgcgactctcaatgccctcgtgatatcaagttcatcaacggattgggcaacgttgagggatgggaaccctccgacagcgacgccaacgcgggcgttggtggaatgggtacttgctgccctgagatggatatctgggaagccaacagcatctccaccgcctacaccccccacccttgcgacagtgtcgagcagaccatgtgcgagggcgattcttgtggcggtacatactccgacgaccgctacggcggtacctgcgaccctgacggttgcgacttcaactcctaccgtatgggcaacaccagcttctacggccccggcgccattatcgacacctcctcgaaattcactgtcgtgacccagttcatcgccgacggcggttccctttccgagatcaagcgcttctatgtccagaacggcgaggtgatccccaactctgagtccaacatctccggcgtcgaaggcaactccatcacctcggaattctgcactgcccagaaaacagcgttcggcgacgaggacatctttgctcagcacggcggcctcagcgccatgggcgatgccgcatccgccatggttctcatcctcagtatctgggatgaccaccactccagcatgatgtggcttgacagctcttaccccaccgatgctgacccaagccagcctggtgttgcccgtggcacttgcgagcagggtgctggtgaccctgatgttgttgagtctgagcacgccgatgcctctgtgaccttctccaacatcaagttcggtccgatcggctccaccttttaa

SEQ ID NO: 23 - DNA, 2657 bp, Artificial Sequence
  Synthesized Beta-glucosidase Bgl1 (Bgl3A) Trichoderma spp
cggcctcaaatcgtaaatctcggtgtgggtaggagtgcaacgatgggatttggccgcaatgctgccgagcccgagtgtttctgcaacgttatccaggagatttgcgcttgcccaagagggagttgacggggagagtcccaactggttccttcagtaacgccaccctggcagactatataacttgtggacaagactctgctttgttgagttcttcctaccagtcttgaccaagaccattctgttgagcccaatcagaaatgcgttaccgaacagcagctgcgctggcacttgccactgggccctttgctagggcagacagtcagtatagctggtccatactgggatgtatatgtatcctggagacaccatgctgactcttgaatcaaggtagctcaacatcgggggcctcggctgaggcagttgtacctcctgcagggactccatggggaaccgcgtacgacaaggcgaaggccgcattggcaaagctcaatctccaagataaggtcggcatcgtgagcggtgtcggctggaacggcggtccttgcgttggaaacacatctccggcctccaagatcagctatccatcgctatgccttcaagacggacccctcggtgttcgatactcgacaggcagcacagcctttacgccgggcgttcaagcggcctcgacgtgggatgtcaatttgatccgcgaacgtggacagttcatcggtgaggaggtgaaggcctcggggattcatgtcatacttggtcctgtggctgggccgctgggaaagactccgcagggcggtcgcaactgggagggcttcggtgtcgatccatatctcacgggcattgccatgggtcaaaccatcaacggcatccagtcggtaggcgtgcaggcgacagcgaagcactatatcctcaacgagcaggagctcaatcgagaaaccatttcgagcaacccagatgaccgaactctccatgagctgtatacttggccatttgccgacgcggttcaggccaatgtcgcttctgtcatgtgctcgtacaacaaggtcaataccacctgggcctgcgaggatcagtacacgctgcagactgtgctgaaagaccagctggggttcccaggctatgtcatgacggactggaacgcacagcacacgactgtccaaagcgcgaattctgggcttgacatgtcaatgcctggcacagacttcaacggtaacaatcggctctggggtccagctctcaccaatgcggtaaatagcaatcaggtccccacgagcagagtcgacgatatggtgactcgtatcctcgccgcatggtacttgacaggccaggaccaggcaggctatccgtcgttcaacatcagcagaaatgttcaaggaaaccacaagaccaatgtcagggcaattgccagggacggcatcgttctgctcaagaatgacgccaacatcctgccgctcaagaagcccgctagcattgccgtcgttggatctgccgcaatcattggtaaccacgccagaaactcgccctcgtgcaacgacaaaggctgcgacgacggggccttgggcatgggttggggttccggcgccgtcaactatccgtacttcgtcgcgccctacgatgccatcaataccagagcgtcttcgcagggcacccaggttaccttgagcaacaccgacaacacgtcctcaggcgcatctgcagcaagaggaaaggacgtcgccatcgtcttcatcaccgccgactcgggtgaaggctacatcaccgtggagggcaacgcgggcgatcgcaacaacctggatccgtggcacaacggcaatgccctggtccaggcggtggccggtgccaacagcaacgtcattgttgttgtccactccgttggcgccatcattctggagcagattcttgctcttccgcaggtcaaggccgttgtctgggcgggtcttccttctcaggagagcggcaatgcgctcgtcgacgtgctgtggggagatgtcagcccttctggcaagctggtgtacaccattgcgaagagccccaatgactataacactcgcatcgtttccggcggcagtgacagcttcagcgagggactgttcatcgactataagcacttcgacgacgccaatatcacgccgcggtacgagttcggctatggactgtgtaagtttgctaacctgaacaatctattagacaggttgactgacggatgactgtggaatgatagcttacaccaagttcaactactcacgcctctccgtcttgtcgaccgccaagtctggtcctgcgactggggccgttgtgccgggaggcccgagtgatctgttccagaatgtcgcgacagtcaccgttgacatcgcaaactctggccaagtgactggtgccgaggtagcccagctgtacatcacctacccatcttcagcacccaggacccctccgaagcagctgcgaggctttgccaagctgaacctcacgcctggtcagagcggaacagcaacgttcaacatccgacgacgagatctcagctactgggacacggcttcgcagaaatgggtggtgccgtcggggtcgtttggcatcagcgtgggagcgagcagccgggatatcaggctgacgagcactctgtcggtagcgtagcgcgaggagggtgaaggcggttgacctgtgac

SEQ ID NO: 24 - DNA, 2171 bp, Artificial Sequence
  Synthesized Beta-glucosidase Bgl2 (Bgl1A) Trichoderma spp
agtacttgcatcttagtctcttcaaaggttgcaaacagccaccctccccatgcaacttgtcccttgggtaaaatggtcatggccagctggctaaatgcgcaggtacatactaataatctgcacacagttgagccatccgggaaaatgcagggtctatcgcaatcagcagcccatcattgtatgtatttctccatgttaattttccgaaagttgggggtggtggtgagggggcgttgtctataatgtgtgtaccttatatatatatatccggggaccaacgaacacgctattctcttgtctgtgtctgtcctctgatttaggaagaggattctgcatgcctgagttggagttgattatctgagtgaggagttgcgtgtttgtctttttcaaagagcttggtcattgacacgctttgtttttatctcgaacttgcttacggtccgaagcacgaacaacaacaagacggagaacaaagagaagaggaagaagaagaagaagaaggaagaagaaggaaaagaagaaagaaagaaggaaaaaaaccgagagacggcaagcatgttgcccaaggactttcagtgggggttcgccacggctgcgtacgttgttgcatcatgctcccttcaaacgccccccccctgatagtccatctcacttacacccgtccgacagctaccagatcgagggcgccgtcgaccaggacggccgcggccccagcatctgggacacgttctgcgcgcagcccggcaagatcgccgacggctcgtcgggcgtgacggcgtgcgactcgtacaaccgcacggccgaggacattgcgctgctcaagtcgctcggggccaagagctaccgcttctccatctcgtggtcgcgcatcatccccgagggcggccgcggcgatgccgtcaaccaggcgggcatcgaccactacgtcaagttcgtcgacgacctgctcgacgccggcatcacgcccttcatcaccctcttccactgggacctgcccgagggcctgcatcagcggtacggggggctgctgaaccgcaccgagttcccgctcgactttgaaaactacgcccgcgtcatgttcagggcgctgcccaaggtgcgcaactggatcaccttcaacgagccgctgtgctcggccatcccgggctacggctccggcaccttcgcccccggccggcagagcacctcggagccgtggaccgtcggccacaacatcctcgtcgcccacggccgcgccgtcaaggcgtaccgcgacgacttcaagcccgccagcggcgacggccagatcggcatcgtcctcaacggcgacttcacctacccctgggacgccgccgacccggccgacaaggaggcggccgagcggcgcctcgagttcttcacggcctggttcgcggatcccatctacttgggcgactacccggcgtcgatgcgcaagcagctgggcgaccggctgccgacctttacgcccgaggagcgcgccctcgtccacggctccaacgacttttacggcatgaaccactacacgtccaactacatccgccaccgcagctcgcccgcctccgccgacgacaccgtcggcaacgtcgacgtgctcttcaccaacaagcagggcaactgcatcggccccgagacgcagtccccctggctgcgcccctgtgccgccggcttccgcgacttcctggtgtggatcagcaagaggtacggctacccgcccatctacgtgacggagaacggcacgagcatcaagggcgagagcgacttgcccaaggagaagattctcgaagatgacttcagggtcaagtactataacgagtacatccgtgccatggttaccgccgtggagctggacggggtcaacgtcaaggggtactttgcctggtcgctcatggacaactttgagtgggcggacggctacgtgacgaggtttggggttacgtatgtggattatgagaatgggcagaagcggttccccaagaagagcgcaaagagcttgaagccgctgtttgacgagctgattgcggcggcgtgatggaaaaagtttttgagttgatgatacctgcaaagggatgctgaagatgatgatgatgtatgaacacgaaagggatagtattatagcaagctaggataacctgatgcttcttcaaaggggaaattgatatataaacatgcat

SEQ ID NO: 25 - DNA, 2625 bp, Artificial Sequence
  Synthesized Beta-glucosidase Bgl3 Cel3b (Bgl3B) Trichoderma spp
atgaagacgttgtcagtgtttgctgccgcccttttggcggccgtagctgaggccaatccctacccgcctcctcactccaaccaggcgtactcgcctcctttctacccttcgccatggatggaccccagtgctccaggctgggagcaagcctatgcccaagctaaggagttcgtctcgggcttgactctcttggagaaggtcaacctcaccaccggtgttggctggatgggtgagaagtgcgttggaaacgttggtaccgtgcctcgcttgggcatgcgaagtctttgcatgcaggacggccccctgggtctccgattcaacacgtacaacagcgctttcagcgttggcttgacggccgccgccagctggagccgacacctttgggttgaccgcggtaccgctctgggctccgaggcaaagggcaagggtgtcgatgttcttctcggacccgtggctggccctctcggtcgcaaccccaacggaggccgtaacgtcgagggtttcggctcggatccctatctggcgggtttggctctggccgataccgtgaccggaatccagaacgcgggcaccatcgcctgtgccaagcacttcctcctcaacgagcaggagcatttccgccaggtcggcgaagctaacggttacggataccccatcaccgaggctctgtcttccaacgttgatgacaagacgattcacgaggtgtacggctggcccttccaggatgctgtcaaggctggtgtcgggtccttcatgtgctcgtacaaccaggtcaacaactcgtacgcttgccaaaactccaagctcatcaacggcttgctcaaggaggagtacggtttccaaggctttgtcatgagcgactggcaggcccagcacacgggtgtcgcgtctgctgttgccggtctcgatatgaccatgcctggtgacaccgccttcaacaccggcgcatcctactttggaagcaacctgacgcttgctgttctcaacggcaccgtccccgagtggcgcattgacgacatggtgatgcgtatcatggctcccttcttcaaggtgggcaagacggttgacagcctcattgacaccaactttgattcttggaccaatggcgagtacggctacgttcaggccgccgtcaatgagaactgggagaaggtcaactacggcgtcgatgtccgcgccaaccatgcgaaccacatccgcgaggttggcgccaagggaactgtcatcttcaagaacaacggcatcctgccccttaagaagcccaagttcctgaccgtcattggtgaggatgctggcggcaaccctgccggccccaacggctgcggtgaccgcggctgtgacgacggcactcttgccatggagtggggatctggtactaccaacttcccctacctcgtcacccccgacgcggccctgcagagccaggctctccaggacggcacccgctacgagagcatcctgtccaactacgccatctcgcagacccaggcgctcgtcagccagcccgatgccattgccattgtctttgccaactcggatagcggcgagggctacatcaacgtcgatggcaacgagggcgaccgcaagaacctgacgctgtggaagaacggcgacgatctgatcaagactgttgctgctgtcaaccccaagacgattgtcgtcatccactcgaccggccccgtgattctcaaggactacgccaaccaccccaacatctctgccattctgtgggccggtgctcctggccaggagtctggcaactcgctggtcgacattctgtacggcaagcagagcccgggccgcactcccttcacctggggcccgtcgctggagagctacggagttagtgttatgaccacgcccaacaacggcaacggcgctccccaggataacttcaacgagggcgccttcatcgactaccgctactttgacaaggtggctcccggcaagcctcgcagctcggacaaggctcccacgtacgagtttggcttcggactgtcgtggtcgacgttcaagttctccaacctccacatccagaagaacaatgtcggccccatgagcccgcccaacggcaagacgattgcggctccctctctgggcagcttcagcaagaaccttaaggactatggcttccccaagaacgttcgccgcatcaaggagtttatctacccctacctgagcaccactacctctggcaaggaggcgtcgggtgacgctcactacggccagactgcgaaggagttcctccccgccggtgccctggacggcagccctcagcctcgctctgcggcctctggcgaacccggcggcaaccgccagctgtacgacattctctacaccgtgacggccaccattaccaacacgggctcggtcatggacgacgccgttccccagctgtacctgagccacggcggtcccaacgagccgcccaaggtgctgcgtggcttcgaccgcatcgagcgcattgctcccggccagagcgtcacgttcaaggcagacctgacgcgccgtgacctgtccaactgggacacgaagaagcagcagtgggtcattaccgactaccccaagactgtgtacgtgggcagctcctcgcgcgacctgccgctgagcgcccgcctgccatga

SEQ ID NO: 26 - DNA, 2502 bp, Artificial Sequence
  Synthesized Beta-glucosidase Bgl4 (Bgl3C) Trichoderma spp
atggctgatattgatgttgaggccatcttgaagaagctcaccctggccgagaaggtcgatctgctggctggtatcgacttctggcacacaaaggctctccccaagcatggagtcccctctctccgctttacagatggccccaacggcgtaagagggaccaagttcttcaatggcgtccctgcggcctgcttcccttgcggcacgtcgctcggttccacattcaaccaaactctgctcgaagaggcaggtaagatgatgggcaaagaggccatcgctaagagtgcgcatgtgatcctcggcccgactatcaacatgcaacgctcccctctcggtggacgtggcttcgagtcgattggtgaggatccgttcctggcgggcttgggagctgcggctctcatccgcggcattcagagcactggagtgcaggctacgatcaagcactttttgtgcaatgatcaggaggacaggcgcatgatggtgcagagcatcgtcacggagcgggctctccgtgaaatctacgcactcccgttccagattgctgtgcgagactcccagccgggtgcgttcatgacggcgtacaatggcatcaatggcgtgtcgtgcagcgagaaccctaaatatcttgatgggatgcttcgaaaggaatggggttgggatggcctaatcatgagcgactggtacggcacatacagtaccacagaagccgttgtggcaggcctcgacctcgagatgcccggacctccacgcttccgaggagaaacactcaagttcaacgtctccaacggaaagccctttatccacgtcattgaccagagggctagggaagttcttcagttcgtcaagaagtgtgctgcctccggagtgacggagaacggccccgagacgactgtcaacaacacccccgaaacggcagctctcctccggaaggttggcaacgagggcatcgtgctgctgaagaacgagaacaacgttctgcccttgagcaagaagaagaagacgctgattgtcggccccaacgccaagcaggccacataccacggcggaggctctgccgcactcagggcctactacgcagtcactccctttgacggcctcagcaagcagctcgagacgccgccatcgtacaccgtcggcgcctacaccaccgttcctcccattctaggcgagcagtgcctcacgcccgacggcgctccgggcatgcgctggagggtcttcaacgagccccctggtacccctaaccgccagcacattgacgagctcttcttcaccaagacggacatgcacctggtggactactaccaccccaaggcggcagacacgtggtacgccgacatggagggcacgtacaccgccgacgaggactgcacctacgagctcggcctcgtcgtctgcggcacggcaaaggcgtacgtagacgaccagctcgtcgtcgacaacgccaccaagcaggtccccggcgatgccttcttcggctccgccacccgcgaggagacgggccgcatcaatctcgtcaagggcaacacgtacaagttcaagatcgagttcggctccgcacccacctacaccctcaagggcgacaccatcgtccccggccacggctccctccgcgtcggcggctgcaaggtcattgacgaccaggccgaaatcgaaaagtccgtcgccctcgccaaggagcacgaccaggtcatcatctgcgcgggccttaacgccgactgggagaccgagggcgccgaccgcgcgagcatgaagctccccggcgtgctggaccagctcattgccgacgtggccgccgcgaacccaaacaccgtcgtcgtcatgcagacgggcacccccgaggagatgccctggctcgacgccacgcccgccgtcatccaggcctggtacggcggcaacgagacgggcaactccattgccgacgtcgtctttggcgactacaacccctcgggcaagctgtccctcagcttccccaagcgcctgcaggacaaccccgcgtttctcaacttccgcaccgaggccgggcgcacgctgtacggcgaggacgtctacgtcgggtacaggtactacgagtttgccgacaaggacgtcaatttcccctttggccacggcctgtcctacaccacttttgccttttccaatctctccgtgtctcacaaggacggcaagctgagcgtgtccctctccgtgaagaacaccggctccgtgcccggcgcacaggtggcccagctctacgtcaagcccctccaagcggccaagattaaccgccccgtcaaggagctcaagggcttcgcaaaggtcgaactgcagcccggcgagacgaaggcggtgacaatcgaggagcaggagaagtacgtcgctgcgtattttgatgaggagcgggatcagtggtgtgtcgaaaagggtgactatgaggttatcgtgagcgacagcagcgcagcgaaggatggggttgcgctcaggggtaagtttacggtgggagagacgtattggtggtctggcgtgtaa

SEQ ID NO: 27 - DNA, 1455 bp, Artificial Sequence
  Synthesized Beta-glucosidase Bgl5 Cel1b (Bgl1B) Trichoderma spp
atgcccgagtcgctagctctgcccaacgactttgaatggggcttcgcaacggccgcctaccagatcgaaggcgccgtcaaagaaggtggccgcggcccgtccatctgggacacgtactgccacctggagccatcgcgcaccaacggcgccaacggcgatgtggcttgcgatcactaccaccgctacgatgaggactttgatctcttgaccaagtacggcgcaaaggcctaccgcttctccttgtcgtggtcgcggatcattcccctcggcggcaggctggatcccgtcaacgaggagggaattgagttttacagcaaactgattgacgccctgttgaggcggggtatcacgccttgggtgactttgtaccactgggatctgcctcaggcgcttcacgatcgctatggaggctggctcaacgtggaagaggtccagctggactttgagcggtatgcgaggttgtgctttgaacgttttggggaccgagtccagaactggatcaccatcaacgawccctggattcaggccatctatggatatgccaccggcagcaacgccccgggcaggagcagcattaacaagcactccaccgagggcaacactgccactgagccgtggctcgctggaaaggcccagatcatgagccatgcccgcgccgtggccgtctacagcagggactttcgcccctcgcaaaagggccagatcggcatctcgctcaacggcgactactatgagccctgggacagcaatgagcctcgggacaaggaggctgctgagcgacggatggaatttcacattggctggtttgccaatcccatcttcttgaagaaggactatccagagagcatgaagaagcagctgggcgagaggcttccagccctcactcccgcggactttgccatcctcaatgccggagagaccgacttctacggcatgaattactacacatcccagttcgcgcgccacctagacggtcccgtccccgagacggactatctcggcgccatccatgagcaccaggagaataaggacggcagccccgttggcgaggagagcggcctcgcctggctgcgctcctgcccggacatgttccggaagcatctcgcccgggtgtacggcctgtacggcaagcccatctacatcaccgagaacggatgcccgtgccctggagaggagaacatgacgtgcgaggaggccgtcaacgaccccttccgcatccgstactttgactcgcacttggactcgatttccaaggccattacccaggacggcgtcgtcgtcaaggggtactttgcgtgggcgttgctcgataacttggaatggtcagatggctacggacccagattcggcgtcacgttcacagactacaccaccctcaagcgcacgcccaagaagtctgccctggtcctcaaggacatgtttgcggcccggcagagggttaaagtggcggcataa

SEQ ID NO: 28 - DNA, 2517 bp, Artificial Sequence
  Synthesized Beta-glucosidase Bgl6 Trichoderma spp
atgggcgaatggcaggagcagatgatgggttttgacgtggaggatgttctgtctcagctgagccaaaatgagaagattgctctcttgtccggcattgatttctggcatacttatcccataccaaagtacaacgtcccttcagtccgcctaacggacggtcctaacggcatacgaggcacaaagttttttgctggcattcctgctgcctgcctgccatgtgggacggccctggcctctacctgggataagcagctgctgaagaaggctgggaagctgctcggtgatgagtgcatcgcaaaaggcgcccactgctggctgggcccaacaatcaatactccccgatctcctctgggggggcgcggcttcgagtcattttcggaagatccgtacctgtccggcatccttgctgcatctatgattctcggctgtgaaagcacaggtgtcatctctgccgtcaaacactttgtcgccaacgaccaggagcacgagcggcgagcggtcgactgtctcatcacccagcgggctctccgggaggtctatctgcgacccttccagatcgtagcccgagatgcaaggcccggcgcattgatgacatcctacaacaaggtcaatggcaagcacgtcgctgacagcgccgagttccttcagggcattctccggactgagtggaattgggatcctctcattgtcagcgactggtacggcacctacaccactattgatgccatcaaagccggccttgatctcgagatgccgggcgtttcacgatatcgcggcaaatacatcgagtctgctctgcaggcccgtttgctgaagcagtccactatcgatgagcgcgctcgccgcgtgctcaggttcgcccagaaggccagccatctcaaggtctccgaggtagagcaaggccgtgacttcccagaggatcgcgtcctcaaccgtcagatctgcggcagcagcattgtcctactgaagaatgagaactccatcttacctctccccaagtccgtcaagaaggtcgcccttgttggatcccacgtgcgtctaccggctatctcgggaggaggcagcgcctctcttgtcccttactatgccatatctctatacgatgccgtctctgaggtactagccggtgccacgatcacgcacgaggtcggtgcctatgcccaccaaatgctgcccgtcatcgacgcaatgatcagcaacgccgtaatccacttctacaacgaccccatcgatgtcaaagacagaaagctccttggcagtgagaacgtatcgtcgacatcgttccagctcatggattacaacaacatcccaacgctcaacaaggccatgttctggggtactctcgtgggcgagtttatccctaccgccacgggaatttgggaatttggcctcagtgtctttggcactgccgacctttatattgataatgagctcgtgattgaaaatacaacacatcagacgcgtggtaccgcctttttcggaaagggaacgacggaaaaagtcgctaccaggaggatggtggccggcagcacctacaagctgcgtctcgagtttgggtctgccaacacgaccaagatggagacgaccggtgttgtcaactttggcggcggtgccgtacacctgggtgcctgtctcaaggtcgacccacaggagatgattgcgcgggccgtcaaggccgcagccgatgccgactacaccatcatctgcacgggactcagcggcgagtgggagtctgagggttttgaccggcctcacatggacctgccccctggtgtggacaccatgatctcgcaagttcttgacgccgctcccaatgctgtagtcgtcaaccagtcaggcaccccagtgacaatgagctgggctcataaagcaaaggccattgtgcaggcttggtatggtggtaacgagacaggccacggaatctccgatgtgctctttggcaacgtcaacccgtcggggaaactctccctatcgtggccagtcgatgtgaagcacaacccagcatatctcaactacgccagcgttggtggacgggtcttgtatggcgaggatgtttacgttggctacaagttctacgacaaaacggagagggaggttctgtttccttttgggcatggcctgtcttacgctaccttcaagctcccagattctaccgtgaggacggtccccgaaaccttccacccggaccagcccacagtagccattgtcaagatcaagaacacgagcagtgtcccgggcgcccaggtcctgcagctatacatttcggccccaaactcgcctacacatcgcccggtcaaggagctgcacggattcgaaaaggtgtatcttgaagctggcgaggagaaggaggtacaaatacccattgaccagtacgctactagcttctgggacgagattgagagcatgtggaagagcgagaggggcatttatgatgtgcttgtaggattctcgagtcaggaaatctcgggcaaggggaagctgattgtgcctgaaacgcgattctggatggggctgtag

SEQ ID NO: 29 - DNA, 2812 bp, Artificial Sequence
  Synthesized BGL6 Beta-Glucosidase
gatcacacccctcccacccttctcttttcaaggttgtccccttctcccacggctttatgtacttcccactctmtaattcgctctttccattccaagccaagcaacatctgtgagcagctcatccttcccaatatgggcgaatggcaggagcagatgatgggttttgacgtggaggatgttctgtctcagctgagccaaaatgagaagattgctctcttgtccggcattgatttctggcatacttatcccataccaaagtacaacgtcccttcagtccgcctaacggacggtcctaacggcatacgaggcacaaagttttttgctggcattcctgctgcctgcctgccatgtgggacggccctggcctctacctgggataagcagctgctgaagaaggctgggaagctgctcggtgatgagtgcatcgcaaaaggcgcccactgctggctgggcccaacaatcaatactccccgatctcctctgggggggcgcggcttcgagtcattttcggaagatccgtacctgtccggcatccttgctgcatctatgattctcggctgtgaaagcacaggtgtcatctctgccgtcaaacactttgtcgccaacgaccaggagcacgagcggcgagcggtcgactgtctcatcacccagcgggctctccgggaggtctatctgcgacccttccagatcgtagcccgagatgcaaggcccggcgcattgatgacatcctacaacaaggtcaatggcaagcacgtcgctgacagcgccgagttccttcagggcattctccggactgagtggaattgggatcctctcattgtcagcgactggtacggcacctacaccactattgatgccatcaaagccggccttgatctcgagatgccgggcgtttcacgatatcgcggcaaatacatcgagtctgctctgcaggcccgtttgctgaagcagtccactatcgatgagcgcgctcgccgcgtgctcaggttcgcccagaaggccagccatctcaaggtctccgaggtagagcaaggccgtgacttcccagaggatcgcgtcctcaaccgtcagatctgcggcagcagcattgtcctactgaagaatgagaactccatcttacctctccccaagtccgtcaagaaggtcgcccttgttggatcccacgtgcgtctaccggctatctcgggaggaggcagcgcctctcttgtcccttactatgccatatctctatacgatgccgtctctgaggtactagccggtgccacgatcacgcacgaggtcggtgcctatgcccaccaaatgctgcccgtcatcgacgcaatgatcagcaacgccgtaatccacttctacaacgaccccatcgatgtcaaagacagaaagctccttggcagtgagaacgtatcgtcgacatcgttccagctcatggattacaacaacatcccaacgctcaacaaggccatgttctggggtactctcgtgggcgagtttatccctaccgccacgggaatttgggaatttggcctcagtgtctttggcactgccgacctttatattgataatgagctcgtgattgaaaatacaacacatcagacgcgtggtaccgcctttttcggaaagggaacgacggaaaaagtcgctaccaggaggatggtggccggcagcacctacaagctgcgtctcgagtttgggtctgccaacacgaccaagatggagacgaccggtgttgtcaactttggcggcggtgccgtacacctgggtgcctgtctcaaggtcgacccacaggagatgattgcgcgggccgtcaaggccgcagccgatgccgactacaccatcatctgcacgggactcagcggcgagtgggagtctgagggttttgaccggcctcacatggacctgccccctggtgtggacaccatgatctcgcaagttcttgacgccgctcccaatgctgtagtcgtcaaccagtcaggcaccccagtgacaatgagctgggctcataaagcaaaggccattgtgcaggcttggtatggtggtaacgagacaggccacggaatctccgatgtgctctttggcaacgtcaacccgtcggggaaactctccctatcgtggccagtcgatgtgaagcacaacccagcatatctcaactacgccagcgttggtggacgggtcttgtatggcgaggatgtttacgttggctacaagttctacgacaaaacggagagggaggttctgtttccttttgggcatggcctgtcttacgctaccttcaagctcccagattctaccgtgaggacggtccccgaaaccttccacccggaccagcccacagtagccattgtcaagatcaagaacacgagcagtgtcccgggcgcccaggtcctgcagctatacatttcggccccaaactcgcctacacatcgcccggtcaaggagctgcacggattcgaaaaggtgtatcttgaagctggcgaggagaaggaggtacaaatacccattgaccagtacgctactagcttctgggacgagattgagagcatgtggaagagcgagaggggcatttatgatgtgcttgtaggattctcgagtcaggaaatctcgggcaaggggaagctgattgtgcctgaaacgcgattctggatggggctgtagattcaacacgtgagcaaaagcgattgcggaaagtaccagaaaagccaagggagtcaaaggatgggaacttgtgtcaatagaagatatgcatgatgggcatttgggatggtgtttggcattatgcaaagaagcaaagatggagtgataaaaaaaaaaaaaaaaa

SEQ ID NO: 30 - DNA, 2298 bp, Artificial Sequence
  Synthesized Beta-glucosidase Bgl7 Trichoderma spp
atgcggctgtgtgacttatccagccttgcgagctgggtcctcgtgacagtagctctgccttcgagcggcgctgctgccaaaggcgtctcgcaaataccttcaacacattcaagtcaaagcaaaggaaatggaccgtgggctcacgcgtatcgtcgcgccgagaagttagtgcgacaaatgacactcgaagaaaaggccaacatcacgcgcggattcaccggcgacaatgtctgtgccggcaacactggctctgttcctcgcctgggatggcccggcatgtgtgtccacgatgccggcaacggagttcgcgcaaccgacttggtcaattcttatccctctggcatccacgtcggggcgagctgggatcgaaacctgacgtacgagagggggcttcatatgggcggggagttcaaagcaaaaggagtcaacgtcccactcggtcccaatgctggcccgctagggcgaacacctctgggtggtcgaaactgggagggtttctccatcgatccgtatctctctggccaattgaacgcagagacaatcactggaatgcaagatgccggagtgattgcgaacatcaagcatttcatcgccaacgaacaagagacgcttcggcgtccctactttggtgtcgaagctgtttctgcaaatatcgatgacagaaccctacacgaatactatctctggccctttatggatagtgtgcatgctggcgtgggatccgtcatgtgctcctataacaggatcaacaacacgtacggatgcatgaacgacaagcttatgaacggaattctcaaggctgaattgggctttcaaggtttcgtcatgcttgactggaatgctcagcacgatctgcaaagcgccaatgccggactcgacatggtgatgcccctcggtggttcttggggcaagaatctgacagatgctgttgcaaacgggacggtcagcgagtctcggattacggacatggccacgaggatcattgctgcatggtacttagtcggtcaagatggcaacaactttccagtaccgggcatcggcttgaaacagctcacgaaaccgcacgagcaagtcgacgcacgcgatcccgcatcgaagcccgtgcttctggagggcgccattgcaggacacgttctagtcaagaacgaaaacaatgcgctaccgttcaacaagaagctaaccatgatctccgtctttggctacgatgctacgatcccacgcacaaagaataccgacattcttttccagctcggatatacctcttcgccggagatggctcaggccgtacttggcaatgaggcgcatttcgaccaggcagcaaagggagggacaattatgactggcgggcgagctggcgcaaacgctccatcatacatcgacgatccgcttgctgctatccaacgtcgagcccgcaaagatgatacttgggtaaattgggacctggactccttcaatccggaagtcaatgctgcttcagatgcttgcttggtcttcatcaatgccatcgcaacagagggctgggaccgtgacggcctccatgacgattttagtgacggccttgtcttgaatgtagccgccaactgctccaacacgattgtcgtcgttcacgccgcgggcactcgcctggttgaccaatggattgagcatcccaatgttactgccgccgtcatcgcgcatcttccaggccaggacagcggtagagccctcgtgaagcttctttatggcgaagccaacttctctggcaaacttccctatacaattgccaagaacgagagcgattactcagtttacaccccatgccagcgacgctctcccgaagacaccgatccccagtgcgatttcaccgaaggcgtctatctcgattatcgcgcttttgatgcgaacaacatgactccccgcttcgagttcggatacgggctcagctacacgtcgttcaattactcagctctctccatcaaaaaggcaaagggccttcggcagtcaaggtgtaccgacgatctttggcaagccgctgcacaagtcaccgcaagcatcaccaacagtggcggcatgtctggaagtgaggttgcgcagctgtacttggccattccaaatagcccgccaaagcaattgcgcggattcaacaaactgttgctgcgtccacatgagtctggaactgttcactttggactcacgaagcgagacttaagtgtttgggatgttgtttctcagtcgtgggttattcaggagggtgagtacaaggtatttgttggggcgagcagccgcgatattcgactcagtggaaaactgcatatttag

SEQ ID NO: 31 - DNA, 2377 bp, Artificial Sequence
  Synthesized BGL7 Beta-Glucosidase
gtagtagcaaacacgtcgcccatcaacatgcggctgtgtgacttatccagccttgcgagctgggtcctcgtgacagtagctctgccttcgagcggcgctgctgccaaaggcgtctcgcaaataccttcaacacattcaagtcaaagcaaaggaaatggaccgtgggctcacgcgtatcgtcgcgccgagaagttagtgcgacaaatgacactcgaagaaaaggccaacatcacgcgcggattcaccggcgacaatgtctgtgccggcaacactggctctgttcctcgcctgggatggcccggcatgtgtgtccacgatgccggcaacggagttcgcgcaaccgacttggtcaattcttatccctctggcatccacgtcggggcgagctgggatcgaaacctgacgtacgagagggggcttcatatgggcggggagttcaaagcaaaaggagtcaacgtcccactcggtcccaatgctggcccgctagggcgaacacctctgggtggtcgaaactgggagggtttctccatcgatccgtatctctctggccaattgaacgcagagacaatcactggaatgcaagatgccggagtgattgcgaacatcaagcatttcatcgccaacgaacaagagacgcttcggcgtccctactttggtgtcgaagctgtttctgcaaatatcgatgacagaaccctacacgaatactatctctggccctttatggatagtgtgcatgctggcgtgggatccgtcatgtgctcctataacaggatcaacaacacgtacggatgcatgaacgacaagcttatgaacggaattctcaaggctgaattgggctttcaaggtttcgtcatgcttgactggaatgctcagcacgatctgcaaagcgccaatgccggactcgacatggtgatgcccctcggtggttcttggggcaagaatctgacagatgctgttgcaaacgggacggtcagcgagtctcggattacggacatggccacgaggatcattgctgcatggtacttagtcggtcaagatggcaacaactttccagtaccgggcatcggcttgaaacagctcacgaaaccgcacgagcaagtcgacgcacgcgatcccgcatcgaagcccgtgcttctggagggcgccattgcaggacacgttctagtcaagaacgaaaacaatgcgctaccgttcaacaagaagctaaccatgatctccgtctttggctacgatgctacgatcccacgcacaaagaataccgacattcttttccagctcggatatacctcttcgccggagatggctcaggccgtacttggcaatgaggcgcatttcgaccaggcagcaaagggagggacaattatgactggcgggcgagctggcgcaaacgctccatcatacatcgacgatccgcttgctgctatccaacgtcgagcccgcaaagatgatacttgggtaaattgggacctggactccttcaatccggaagtcaatgctgcttcagatgcttgcttggtcttcatcaatgccatcgcaacagagggctgggaccgtgacggcctccatgacgattttagtgacggccttgtcttgaatgtagccgccaactgctccaacacgattgtcgtcgttcacgccgcgggcactcgcctggttgaccaatggattgagcatcccaatgttactgccgccgtcatcgcgcatcttccaggccaggacagcggtagagccctcgtgaagcttctttatggcgaagccaacttctctggcaaacttccctatacaattgccaagaacgagagcgattactcagtttacaccccatgccagcgacgctctcccgaagacaccgatccccagtgcgatttcaccgaaggcgtctatctcgattatcgcgcttttgatgcgaacaacatgactccccgcttcgagttcggatacgggctcagctacacgtcgttcaattactcagctctctccatcaaaaaggcaaagggccttcggcagtcaaggtgtaccgacgatctttggcaagccgctgcacaagtcaccgcaagcatcaccaacagtggcggcatgtctggaagtgaggttgcgcagctgtacttggccattccaaatagcccgccaaagcaattgcgcggattcaacaaactgttgctgcgtccacatgagtctggaactgttcactttggactcacgaagcgagacttaagtgtttgggatgttgtttctcagtcgtgggttattcaggagggtgagtacaaggtatttgttggggcgagcagccgcgatattcgactcagtggaaaactgcatatttagggagcatagcttattgagcgaattcgtttctcttttcaaaaaaaaaaaaaaa

SEQ ID NO: 32 - DNA, 2298 bp, Artificial Sequence; identical to SEQ ID NO: 30
  Synthesized Beta-glucosidase Cel3E (Bgl3E) Trichoderma spp

SEQ ID NO: 33 - DNA, 2103 bp, Artificial Sequence
  Synthesized Beta-glucosidase Cel3D (Bgl3D) Trichoderma spp
atgattctcggctgtgaaagcacaggtgtcatctctgccgtcaaacactttgtcgccaacgaccaggagcacgagcggcgagcggtcgactgtctcatcacccagcgggctctccgggaggtctatctgcgacccttccagatcgtagcccgagatgcaaggcccggcgcattgatgacatcctacaacaaggtcaatggcaagcacgtcgctgacagcgccgagttccttcagggcattctccggactgagtggaattgggatcctctcattgtcagcgactggtacggcacctacaccactattgatgccatcaaagccggccttgatctcgagatgccgggcgtttcacgatatcgcggcaaatacatcgagtctgctctgcaggcccgtttgctgaagcagtccactatcgatgagcgcgctcgccgcgtgctcaggttcgcccagaaggccagccatctcaaggtctccgaggtagagcaaggccgtgacttcccagaggatcgcgtcctcaaccgtcagatctgcggcagcagcattgtcctactgaagaatgagaactccatcttacctctccccaagtccgtcaagaaggtcgcccttgttggatcccacgtgcgtctaccggctatctcgggaggaggcagcgcctctcttgtcccttactatgccatatctctatacgatgccgtctctgaggtactagccggtgccacgatcacgcacgaggtcggtgcctatgcccaccaaatgctgcccgtcatcgacgcaatgatcagcaacgccgtaatccacttctacaacgaccccatcgatgtcaaagacagaaagctccttggcagtgagaacgtatcgtcgacatcgttccagctcatggattacaacaacatcccaacgctcaacaaggccatgttctggggtactctcgtgggcgagtttatccctaccgccacgggaatttgggaatttggcctcagtgtctttggcactgccgacctttatattgataatgagctcgtgattgaaaatacaacacatcagacgcgtggtaccgcctttttcggaaagggaacgacggaaaaagtcgctaccaggaggatggtggccggcagcacctacaagctgcgtctcgagtttgggtctgccaacacgaccaagatggagacgaccggtgttgtcaactttggcggcggtgccgtacacctgggtgcctgtctcaaggtcgacccacaggagatgattgcgcgggccgtcaaggccgcagccgatgccgactacaccatcatctgcacgggactcagcggcgagtgggagtctgagggttttgaccggcctcacatggacctgccccctggtgtggacaccatgatctcgcaagttcttgacgccgctcccaatgctgtagtcgtcaaccagtcaggcaccccagtgacaatgagctgggctcataaagcaaaggccattgtgcaggcttggtatggtggtaacgagacaggccacggaatctccgatgtgctctttggcaacgtcaacccgtcggggaaactctccctatcgtggccagtcgatgtgaagcacaacccagcatatctcaactacgccagcgttggtggacgggtcttgtatggcgaggatgtttacgttggctacaagttctacgacaaaacggagagggaggttctgtttccttttgggcatggcctgtcttacgctaccttcaagctcccagattctaccgtgaggacggtccccgaaaccttccacccggaccagcccacagtagccattgtcaagatcaagaacacgagcagtgtcccgggcgcccaggtcctgcagctatacatttcggccccaaactcgcctacacatcgcccggtcaaggagctgcacggattcgaaaaggtgtatcttgaagctggcgaggagaaggaggtacaaatacccattgaccagtacgctactagcttctgggacgagattgagagcatgtggaagagcgagaggggcatttatgatgtgcttgtaggattctcgagtcaggaaatctcgggcaaggggaagctgattgtgcctgaaacgcgattctggatggggctgtag

SEQ ID NO: 34 - DNA, 2502 bp, Artificial Sequence
  Synthesized b-Glucosidase AN2227.2 Aspergillus nidulans
atgcctcaattggatgtggacaagacgattgaagaacttcgtctcggagagaagatcgatcttgtgtcagggatcgacttctggcatactgcatctgtccccaggcttaacattcccagcctgcgcatgtccgatggccccaatggagtccgcggaacgcggttttttaacggtgtgccagcagcatgtttcccctgcgctactgccctgggagcaacctgggacactgaattactgcacaaggtagggcatttgatgggggaagaggccattgccaagggcgcgcacgtcatattgggtcccacaatcaatacgcaacgatcgccgctgggcggccgagggttcgagtctttcgccgaagatggtgttcttgccggccacttggctggctactgttccaagggaatccaggaaaagggcgttgccgcgtgcctgaagcactttgtgtgcaatgaccaggagcatgagcgtttggcggtcgattcgattgttaccgaccgggctacacgcgagatatacctcttgccgttccagattgcgatgaggatttgcaaaacggccacagttatgacggcttataacaagattaacggtacccatgtgagtgagaataagaagtacatcacagacattttgcgcaaggagtggggatgggacggactagttatgagtgactgtacgtcagaatcaataattgccggtctcgacatagagatgccaggtaagacacgctggcggggagacgccctggcacacgccgtatcatccaacaaagtccacgagtttgtcctggatgaacgtgtacgcaacgttctgaaccttgtaaattacgttgagccactgggtatccccgaaaacgcagaggaaaaggttcttaaccgccccgaagaccaagccctactccgacgggctgcggccgagtctatcgtgctcttgaagaacgaggataacatcctacctttcaacaaggagaagtccatcgccgtgatcggaccaaacgccaagatcgccgcttactgtggaggtggctctgcatccttggatgcgtactacacaatcacacccttcgaaggcgtatcagcacagagcaagggtgaggttcacttcgcccaaggttcatactcatacaaggaccttccgttgatcggccacctcctgaagaccgacgacggcaaaacgggcttcaagttccgcgtttacgacgagcccgcatccagtagcaaccgcgaactcctccacgagctacaccttgtctcatcccagggcttcctcatggactatcgccaccctaagatcaagtcttacctttactacgtcgacatggagggctatttcactcccgaagagtccggtgtctacgacttcggcgttgttgtcgtcggaacggggaaactcctcgtggatgatgaagtcgtcgttgataacaccaagaaccagcgtctcggctctgccttctttggaaacggcaccgttgaagaaaagggctccaaggaactcatggctggccagaaatacaaaattaccttccagttcgggacagcacccacctcggacatcgacacccgcggggtggtcatcttcgggcctggaggcttccgcttcggcgctgcccggcgccagacgcaggaagagctcatttccaaagctgtcgaagtcgcctcaaaagcagaccaggttgtcgtcttcgctggtctaacaagcgaatgggaaacagaagggtacgatcgtccggacatggatcttcccccgggttcagacgaactgatttccaaaatcctggaggtgaaacccaacgccgccatcgtcatccagagcggtacgcccgtgacaatgccctgggcgcccaaagccaaggctcttctgcaggcttggttcgggggcaatgagtgtggtaacggcatcgcagatgtgctttatggaaacgtcaacccctctggaaagctgcctttgaccttccccgtccgtttgcaagacaaccctagttacctgaacttccgctcagagcgaggccgcgtcctctacggcgaagacatctatgttgggtaccggtactacgagaaagcccaactcccgccgttattccccttcggccatggcctgtcctacacgactttcacacgcgagaaactggagttgaacacttctcctgaaaaagacaaactgcaagacggagaacccataaccgccagggtgaccgtcacaaacacgggcaaagtcgccggcgccgaaacagtgcagctgtgggttgtgccgcccccgacagaggtgaacagacctgtccgcgaactaaagggttttgcgaaggtgcacctggaacctggagagtcaaaagatgtcgagattgtggttgaaaagaagctggcgacgagctggtgggatgagaaacgggaagcctgggcatcagagaaaggagtttattgggtgcaagttacggggacgggagagggggttctcactgcagagtttgaggttaagaagacgaggttctggaccggattgtga

SEQ ID NO: 35 - DNA, 2517 bp, Artificial Sequence
  Synthesized b-Glucosidase AN2612.2 Aspergillus nidulans
atgggagagatatctcctcgtggtggggatttcgatattgattacattctcgagaatgcctcgtctctggagaagatttccctccttgcgggccatgacttttggcatacagctcccctaccgcgcttcaacgttccctcggtcagagtgagcgatggtcccaacggcgttcgaggcaccaagttcttcgacggcgtccgcgccgcctgtcttccttgtggcaccggcctggccgccacatgggaccagtctttgcttttcgatgccggtgttctcatcggccaagagtgcctagccaaaggtgctcactgctggctaggacccacggtgtgtatccaacggtcgccccttgggggcagaggtttcgagtctttcgcggaggacccatatgccaccggcaaacttgccgccgcctacatccgaggtgcccagtccaccggcgtgatatccactatcaagcatttcgcagcaaacgaccaggagcatgagagaattagcgtcaatgccgtcatgagcgagcgagcgttgcgcgaggttcatttgttgccctttcagatcgctatcgccgatgcagctccgggcgcagtcatgacatgctacaacaagatcaacgggcaacatgtttcggagagcaaggagatgttggatggtctcttgcgcaaggaatggggctggaagggcctcatcatgagtgactggtttggaacgtactcgaccgccgaggctctgaatgccggccttgacctggagatgcctggccccacaaggcttcgaggaccgttactggagcttgcgatttcgagtcgcaaagtctcccgctcaacgcttgacgagcgcgccaggaccgtgctcgaattcgtgaagcgagccaacaaggccgaggtttcgacagtcgagagcacacgagactttcccgaggatcgtaggttgaaccgcaaacttgctgccgatagcattgtcctgcttaagaacgagtcggggcttttgccactaaacctgaaggcgttgaagagtgccgccttgatcgggccaaacatgaagaccgcggcgttctgcgggggaggttctgcgtccctccagccatactacagcatctcgccgtaccagggaatcatgaaccagctccctccgggcgtcgagattatctacgagacaggcgctagttcgtatgtttttatcccggagctggaggcgtcagaagtgcgcacgccggaaggccagcctgggcttcgaatgcgcttctaccgagaaccaccctccgtcaaagaacggcgcgtggttgaggaaaccatcctacaggagtcttcgtggcagctgatgggcttctccaacccacaattggaccggctcttctacgcagacattgaagccgagttgattgctcctgccactggtcctttcgaattcggccttgctgtctatggttctggcagcctctttatcgacgaccagcttataatcgataacactactgtgcaacgagggggcaatttcttcttcggcaaaggcacccgggaggaaaaggccaccgtggatcttgttaagggtcagttgtacaagattagggtggaattcgctagtggcccatcgtccaagctcatgaaaccgggggttgtgaatttcggtggcggcgctggtcgcctgggaatggttcaggccatcgatccggagctggctatcgcccgggccgtcgaggccgcgaagcgtgcagacgtcacgatactgggggttggactgacgcgagaccacgaatccgagggattcgatcggtctcacatggatctgcctcctgccgttgcctccttggtcacagctgtccttgacgtggcgcctgacgccattctcatgacccaaagcggcacgcctttcaacatgcttccttgggcagataatgtgaagacccatctacatgcatggtttggcggcaatgagctaggcaatggcattgcggatgtgttgttcggggtcgtcaatcccagtggaaaacttcctctttctttcccgcggcgaatcgaagacaccccgacatatctcaattttggaagcgagagagggcaagtaacatatggcgagggcatttacgttggttaccgatactacgaaaaagtcctccgagatgtgctctatccctttgggcacggtctgtcgtacacttcctttgcttattccgatttcgctgtcgacaccgcgtccgctacacttaatgttcgtaactcgggagacgtggctggagcagaagtcgtccagctgtatatcgctgccgatgcaacaacgtcgtccatcgcacgtccagtcaaggagctcaaaggatttgcaaaggtgactttgcagccaggtgagacgtgcagtgtgagcataccatttgatcggttcactactgcattttgggatcaggaagcacacgtgtggacgtgcgaaaaagggcagtatagagtcatggttgggtcgagcagccaaaacattctgctggaaggcgtgttggagataaaggagacgacgacctggtcaggcctgtga

SEQ ID NO: 36 - DNA, 2538 bp, Artificial Sequence
  Synthesized b-Glucosidase AN0712.2 Aspergillus nidulans
atgagcttccaaaaggtcgatcctgcgcagatagagagcgtgctctctaagctgactctggaagagaagatctccctgctggcggggaagaacttctgggaaacacaagactatccagaaaagggggtgccacctgtcaagacttcagatggtcccaacggggcgcgcggtgccacattcaagggaggagtaacagctgcctgtttcccagcgtcctctctcttagctgcgacgtgggatctcgacgcggcaaagcacattggagaagcattggctgacgagacgcgctcgaaaggcgcaagggttctattagcacccacagtctgcattcatcgccatcctctgggaggccgcaacttcgaatcgttcagtgaagatcctttccttgcaggaaagctcgcggcgcagtatattaaaggccttcagggcaacggcgtcgctgcaaccatcaagcactatgccgcgaacgagcaagagacctgccgattcacagtcaacgaacatatcacagaacgagcactccgagaaatctacctgaaaccattcgagatcgccatcaaagagtcgaacccgctggcagttatgaccgcctacaacattgtgaatggaacccacgctgactcgaacaattttctcttgcgagatgtgctcaggggagaatggggatggaagggtcttgttatgagtgattggggcggtaccaactccacggccgacgctctgaatgctggtcttgacctagaaatgcccggtcctacaaggtggcgcaaggtagacgaagtgcttgcagtcgtaaagtctggtgccgtgttggaggagactatcgacgagcgcgcgcgcaacgttctggagcttcttgctaagctgaattgtttcgaaaatcctactattccagaagagaaggccatcaatcgtcctgaacaccagaaattaatccgctccgtgggaagtcaaggcctcgtcctcctcaaaaacgagggcgatgttcttcctctgagaaaagagatattgaccaacaagaaggtagctcttctgggctttgcgcgagaagctctcattcacggcggtggcagtgcttcagtcaatgcacattatcgagtcactccagaagagggtctccgcgccgctttaggcgacactgttgagtttgaatatgccaagggtgcacacactttccgccagctgcccttaatgagcgacaacgttgttaacctagaaggccaacctggttggaccctagacttttttgcggacgaggagcccaacggcgagccaggctcatcgatctcgtccgagcaacccagctatataccgctctttgtcaaggaatcatggggaagtgtccgagccagcgcccatttcacacccacgcaaagcgggaaacactattttggcatgtctgggttgggccggtccaaactcttgatcgacggcgaggtcatctacgaacagaagctgaattgtccggactccatgggattcttactcggcggcgtggaagagccagaaatccagtattcattcgaagctggaaagacctatgcggtcgaagtggtttcggtgaaacccaccagcaagggcggtctcgctctgctggacggcttcattggtttccgtctcgggttcatgaccgaggaagagcacaatagagatttgctgtctgaagccgtcgatgtagccaaacgtagtgatatcgcaattgtcttcactggccatactccagattgggagacggaagggcaggaccagataagcttccacctaccgtccaacggttcccaggaccggctcgtggcagctgttggagccgccaaccctaacaccgttgtcgtgaactgcactggcgtagcggttgctatgccttggttggacaaggtcaaggcggttgttcaagcgtggttcccaggacaggaagccggcaatgcaattgctgacgtactcaccggcgctgtgaacccttctggacgtctacctgtgtccttccctcgggcgatcgaggatgcccctgcacacggtaacttccccggtgactacacagacgggaaggacaaccggcgccatctcgaagttacatacaaggaaggtgtcttcgtgggctaccggcactatgatctctctgaagccaaccgcgccaaggtcctgttcccgttcggctacggtctatcctacaccacttttacacatgcgaatcacaaagcaagtgctacgagcaggaacaccgtcgaagtggccgtggatgtcactaacgtcggcacctgtgctggagctgatgttgttcaagtctacgctggtgcaaaactggcagtcccagagaacccagtcaaagagctagttggtttcgccaaggtacacctcaagccgggggagaccaagactgccaatatcacattcgaagtgcgccaactgactcacttcacggagaggagcggcaagtgggagctggaatcaggcgactatgaaatatcaatcggtcagtccgtgagggatatcactggcaaggtggagattggtctggaagcccagaattataaaccttga

SEQ ID NO: 37 - DNA, 1668 bp, Artificial Sequence
  Synthesized b-Glucosidase AN1551.2 Aspergillus nidulans
atgaggggagctatcctggccaccgcggccgccttcgccggcaccgccgttgcggacatgcacatgcgccgccatgcccacgagggtcttcaccaccgtgccctccacgcctcttccgctgttcccgaggaggagtgcggctgcaccaccgaggtcatcacctactggggcgagcctacaactatccccctctcggttcctaccagcaccgtgacctctgagaccaccgagactgttcactctaccagctacagcaccgtcacggtcactgcgacttcctctgccgctccggtcgagactccttccgagactccctctccaacccccgaggtcactctccccaccgccggcgtgaccagctactcggagactggtacctacaccatccctgctaccaccatcaccgtcaccgataccaccactgtctgcggcgctactaccactgagctccccagcggtacccacacctacggtggtgtcaccaccattgtcgagaccgccacgaccatcacctgcccctatgccaccgtcaagcccactggcagcaccgtcaccagcgtcatcgagaccacgacctacgtctgcccctcggctggtacctacaccattgccccgaccaccaccttcgtccctacctccaccgtcgttgtctaccccaccccggaaaccgtcacccctggcacctacaccaaccctggtaccaccatcaccgtgactcgcactgaggacgtctatgtctgcccttacaccaacggcaatgtccccaccagcgtccctgccctgcccaccacctctgctgcctcgaccactactgctgttcccagcagcagcaccaccacctcgtctgccaccagcgtccccaccggtgctagcggaaacaagatgggtatgactttcaccccctacaacaacgacggaagctgcatggccaagaacgatgttcttgagcaggttggtcttatcaaggggaagggattctcccacgttcgtgtttacggaaccgactgccacaccctcgagtacgttggagctgcttgcagcacccacggcctcaagatgatcctcggtgtcaatgtcgagggctccaccggttttgatggtgctcgttcccagttcaaggacattaccaactggggccagtgggatcttgtctctctcattgttgtcggtaacgaagtcgtcactagcaacatcgcgtccgctgcccagctcgccagcttcgtctccgagggcgccagcgctttctctgccgccggctacactggccaggttaccaccgctgagcctatcgacgtctggctcagtaacggtgctactctctgccctgtcgttgatatcctgggtgcgaaccttcaccccttcttcaaccccgagttcaccgccgctgaggccggtactctcgtcagcaaccagatcaaggatctgaagcaggtctgcactggcaaggacgtcattaacctcgagaccggctggcccaacgccggtagtgctaacggcaaggctattcccggtcagtcccagcaaaccactgccatcaagtccctcgtcgagaaggttggcgatgtttccgttttcttctcctacgccgatgacggctggaagtctaagttcgctaccagcgacaaatacaatgttgagcagcactggggatgcatcgaccaattttaa

SEQ ID NO: 38 - DNA, 1857 bp, Artificial Sequence
  Synthesized b-Glucosidase AN1804.2 Aspergillus nidulans
atgagggttgattcaacagttctggccctggtggctctagccaccgactgtctcggcttggctatcaagagcaacgagcctgagctgctacgtcgtgacgcccttcccatttataagaacgcatcatactgtgttgatgagcgtgtgagagacctgttaagtcgcatgacacttgaagagaaagcaggccagctcttccacaagcagctttctgagggacctctcgacgacgacagcagcggcaacagcactgaaacaatgattggcaagaagcacatgacacacttcaatctcgccagcgatatcaccaatgctacgcagacggccgagttcatcaacctgattcagaagcgagcattacagactcgacttgggattcccatcacaatctcgactgatcctcgccattcgttcactgaaaacgtcggcaccgggttccaggccggtgtgttttcacaatggccagagagtctaggtctcgctgcgttgagggaccctcagcttgtcagagaatttgcggaagtagcaagggaggaatacttggctgttggcattcgtgctgcgctgcacccgcaggttgacttgtccacagagccccgatgggcccgtatcagcggtacctggggagaaaacagtacgctgaccagcgagcttattgtcgagtacatcaagggcttccaaggagaaggcaagcttggaccaaaatctgtcaaaactgtcaccaagcacttccctggcggcggcccgatggagaacggcgaggattcgcacttctactatggcaagaaccagacttaccctggaaacaatatcgatgagcacctcataccattcaaggcagcccttgccgcgggtgcaaccgagatcatgccttattactctcgccctatcggcacgaactgggaggctgttgggttctccttcaacaaggagatagtcaccgacttgctccgtggggagctcggatttgatggtatcgtcttgacagactggggtcttatcacggacacttatatcggaaaccagtacatgcccgcgcgtgcctggggcgtggagtatcttagcgagctccaacgcgcagcaagaatcctcgacgcaggttgcgaccaattcggtggcgaggaacggcccgaactcattgttcagcttgtcagagagggcaccatatcagaagatcgaatcgacgtctctgttgctcgactgctcaaggagaagtttctccttggccttttcgataacccatttgtcaatgcctcagctgcgaacaatattgtcggaaacgagcactttgtcaatttaggccgagatgcgcagagaagatcctacacgcttctcaccaacaatcagacaatccttcctctcgccaaacctggtgagggtacaagattctacatcgaaggcttcgattccgccttcatgtccgccaggaactataccgtcgtcaacaccactgaagaagccgactttgccctgctccggtacaatgcgccgtatgaaccacgtaacggtacctttgaggccaatttccacgcaggttctctcgcatttaacgccactgagaaggcacgccaggccaagatctactcatccctcccgactattgtagacatcatcttggaccggcctgccgttattcctgaggtagtcgagcaagcgcaggccgtacttgcgagttacgggagcgacagcgaagccttcttggacgttgtgtttggagtcagcaagccggagggcaaactcccgttcgatttgccccggtcaatggacgctgtcgaggcgcaggcagaggatctgccattcgatacggagaatcctgtcttcaggtatggtcatggcctggagtacgaggataactga

SEQ ID NO: 39 - DNA, 2685 bp, Artificial Sequence
  Synthesized a-/b-Glucosidase AN7345.2 Aspergillus nidulans
atggccggaactctcctctcatggactcccctggctctagctctgatggctagagctctcagtcaattgcctttgaccgactgtcctggatacagggtgataaacgtcgaagaacgacctcgcggactgacagcggatctgacactcgctggtacgccgtgcaacgtgtatggagttgatatcgagaacctgaggctcgaaaccgactatgatacgaatcaaagacttcatgtcaaaatctacgatgcggatgagaacgtctaccaagtgcccgactctgtcttccctaggcccgttgtcaatgaccaagcctgtgctgatgaaaacaccccggagctgaggttttcgtacgcagaagatcctttctccttcgccgtctcccgagcgagcaatgacgaaacactcttcaatactacaggacataatttaatatttcaatcccaatatgtgaatctccgcacctctctcccacaaaaccctaatctctacggacttggcgagcacagcgatccccttcgcctgaacactatcaattatacccgcacactctggaaccgggatgcttataccatccccgcggggacgaatctttatggggcgcacccgatgtatatagaccaccggggcgaggcaggaacgcacggagttttccttttgaattcaaacgggatggacatcaagattgacaaaaactctgataatatccagttcctggaatataatatcctggggggcgttctagacttttatttctttgctgggccaagtccgaaagacgtgagcgtgcagtatgcggaggttgcaggcttaccagccatggttccatactggggtcttggatttcatcaatgccgttacggttacagagatatcttcgaagtcgctgcggtagtacataactatagcgaagcgagaattcccctcgaaacaatgtggaccgatatcgactacatggatcaccgcaaagtttttacactcgatcgagagcgattcccattggatactgtccgagctttggtgcaatatcttcaccagcgagaccaacattatattgttatggttgatcccgcggtagctcactctgagaacggcgctttcacacgagggctagagaaggatgtgttcatgcggaagcaagatggaacgctatatcaaggcgctgtctggcccggtgcgacagtttttcccgactggttccatccaaacacttcagactattggatcaacgaattcgcgttgttcttcaatgcagaatcaggagttgacatcgatgccctgtggatcgacatgaacgaggccgctaacttttgtgactggccctgtacagaccctgtcgcctatgcggaggagaataacctccctccggagccgccagctgtcaggccaaatccgagcagccttccagggtttccggctgagtttcagcctgtaaacagcaataacaataacagtagcagaaaacgcgagacgcaagttgttattgcagccaggcaaggatttgtcaaagtcggaaacgacaatggcaatggcaggaggctgggtctgcagggccgcgaactgatcgatccgccctacaagatcgccaacgcagctgggtcgctgagcaataagactatgaatacagacattttccatgccaatgggcttgctgagtatgatactcacaatctatacgggacaatgatgagttcgctttcacgagacgccatgctctacagacgcccagagaaacggcccctagtgataacgcgcagcacctttgccggtgcaggctcctacgtcggtcactggctcggcgacaacgctagcacctggaccaaataccgcatctccatcgcccaaatgctcgccttcgcatccatattccagatccccatggtcggttctgacgcatgtggttttacaggcaatacgactgaggagctgtgctcgcgctgggcaaccctggccgcttttaaccccttctttcgcaatcacaatgagtacgggatggtctcacaagagttttacaggtggaactctgttgccgaggcagcaaggaaggcaataagcatccgatacagtttgcttgactatctttacacggagttccatgaacagacggtcacgggcgagccgtttttgctcccacttttctttgtctatccaaatgaccccaatgtggtgggtattgactcacagttcttctatggggatgcgattcttgtcagtcctgttattgaagaaggtaagacggaggttcatgcttatttcccgggtgatttgttctatgattggtacacaggtcttcctcttcgtggaaatggtgaggtgataacattgacggacattgggtacactgacatcccgttacatgtacgtggtgggaaaattgtgcctgtacggaccgggtctgcggggatgaatacgacgactgaggtgaggaagagcgggtttcggttggtcattgccccaggactcgatggtagggctgcaggccgcctgtacattgatgatggggaatcgttggagcagactgccatggtggatgtggtctttacctatgaggacggtagggtgagcgttgatggcgtgtttacgctacagactgatctacgtgttgaggctgtgactgtgttcggggacaatgtggtcgaaaggactatcgacctgcctctttccggaccgggaggtgttgagctgtag

SEQ ID NO: 40 - DNA, 2435 bp, Artificial Sequence
  Synthesized Beta-glucosidase Orpinomyces
aaataattaaattaagatatatataataaataaataaaatgaagactcttactgttttctctgctttattagctgttactgctgctaagaagtgcattgttaagagcgatgctgctgttgcttctgaagctgaagaagtcactgctgaacttactgctccagaagattctggtgttgaatctggtgaagatgatgaattattagatttatctaccattgactacggagatgatgttgacatgtctactgttaagaagcttccagctgacttcaaatggggtgctgctactgctgcttaccaagttgaaggtgcctgggatgaagaaggtcgtggtgaatctgtctgggatcacttcactcatctttacccaaagaatgtcgaatctggtgacagatccaaggacttctccactaatggtaacattgcttgtgattcttaccacaagttcgacgaagatgttaaaatgttaaagctcatgaatgctaaatactaccgtttctctatttcatggccacgtcttttcccagatggtcaagccagaaaggttgacggtaaatggaacgtcaatgaaaagggtgctgaatactacgatatggttatcaatactcttcttaaaaacgatattgttccattcgttactctttaccactgggatcttccatacgctctccacgaaaagtatggtggttggttagattaccactcccaagatgatttcgccaaatacgccgaattctgtttcgaacgttttggtgaccgtgtcaagaactggattactattaacgaaccatgggttaactgtgtttctggttaccgtcttggtccaggtaaggctccatacagatgtactggtgaagctccacgtaagctccaaaactccaccgatcttgacttagaaggaggttgttcctacgaaattggtccaactcaatactctaagaactctgaacctcttccagctaaccgtgttccacaaaagttagaagatgtctggtgttcccacaatattcttcttggtcacgctaaggctgttaaggtctaccgtgaaaaattccaaaagaagcaaaagggtcttattggtattaccgttgatggtgaagctcaaattccatgggttgaaccaggtatgaccaagaaggaatacgaaaacaacttaaagtacgccaacttagctgctgaattccgtattggttggtactctgacccaccaatggttggtgactatccaaagtccgttaaggaaagaatgggtaaggacttaccagaattcactgaagaagaaaagaagatcttaaagggatcttcctctgacttcttaggttggaacacctacactgctcactgggctgctcaagctaagaacgaagatggttcttacattcaaccaccaactgccgaagaagctaacttcgacaactccaagaaggatatgtgggatgataactgtaagggacgtggtgatggttggacttgtattccaccaactcttggttcccaagctggttcttcctggaacactaagttcgctccaactatccgtgttggtcttaactggttctccaagcgttacgaaggtttaattaagaacggtatcgttattactgaaaacggttgtgcccaaccaaactacaaggttgctcgtgctaatgatgaagttactaagaagtacttcgaatctattggtcaaccaaagtatgctgatacttacaaggaagaagatattgaaagagaagacaacttagaaggtactcttatgcacgatacctaccgtattgactggtacgaccaataccttaagaaccttcgtcttgcctacgccgtcgataacatcgatgtccgtggttacatggcctggtctttacttgataactttgaatgggaaaacggttacgaaactcgttttggtatgacttacattgacttctacaatgacaaggaaatgaagcgtgttccaaaggattcccttgaacatcttggtcaatggtacctcgaaaatgttgaacaaaactaaatttcttaaaaatttataataatattttattacaattataaataaatatattaataatggaattattttattcacttcttttgctataagtagtgaaataaattaattttataattatataaatttatagaataaatcttttttgaatcattaaaattaaaataaataatatacaaattttaatgaataataatgattattattaaatattctaaagaagatttataatttttaagaataaatataaagcaagaaaacaaatataattaaaaaaaataaaaattaaatataaaataaaaataaaataataaagctttgtgtttaaaataaaatagagtagtaaaagctattcgctattcttaataaatataaaaatataaaataaagttaaaaatttaaataaaataaaaaatattaataaaa

(19) United States
(12) Patent Application Publication
(10) Pub. No.: US 2011/0165635 A1
(43) Pub. Date: Jul. 7, 2011

(54) METHODS AND MATERIALS FOR PROCESSING A FEEDSTOCK

(75) Inventors: Gregory P. Copenhaver, Chapel Hill, NC (US); Daphne Preuss, Chicago, IL (US); Jennifer Mach, Chicago, IL (US)

(73) Assignee: CHROMATIN, INC., Chicago, IL (US)

(21) Appl. No.: 12/989,038

(22) PCT Filed: Apr. 21, 2009
(86) PCT No.: PCT/US2009/041260

§ 371 Date: Mar. 11, 2011

Related U.S. Application Data
(60) Provisional application No. 61/046,705, filed on Apr. 21, 2008.

(51) Int. Cl. C12P 17/04; C12P 1/00; C12P 7/04; C12P 7/16; C12P 7/06; C12P 5/00; C12P 5/02; C12P 3/00; C12P 1/02; C12N 5/10; C12N 1/15; C12N 1/13

(52) U.S. Cl. 435/126; 435/41; 435/157; 435/160; 435/161; 435/166; 435/167; 435/168; 435/171; 435/419; 435/254.11; 435/257.2

(57) ABSTRACT

The present disclosure relates generally to methods for processing a feedstock. Specifically, methods are provided for processing a feedstock by mixing the feedstock with an additive organism that comprises one or more transgenes coding for one or more enzymes. The expressed enzymes may be capable of breaking down cellulosic and lignocellulosic materials and converting them to a biofuel.

### FIELD

The present disclosure relates generally to methods for processing a feedstock. Specifically, methods are provided for processing a feedstock by mixing the feedstock with an additive organism that comprises one or more transgenes coding for one or more enzymes. Enzymes released from the additive organism may be used to manufacture a biofuel or another hydrocarbon or co-product by converting the feedstock into sugars that can be fermented or chemically converted or by extracting oils that may be processed into biodiesel.

### BACKGROUND

The use of biofuels are considered a means for reducing greenhouse gas emissions and increasing energy security by providing an alternative to fossil fuels. Biofuels may be produced from the conversion of a biomass (e.g., trees, grasses, agricultural crops or other biological material) into liquid or gaseous fuels (e.g., ethanol, propanol, butanol, methanol, methane, 2,5-dimethylfuran, dimethyl ether, biodiesel, biogasoline, paraffins, other hydrocarbons or co-products or hydrogen) by converting the biomass into sugars that can be fermented or chemically converted to form a biofuel, or otherwise extracting oils from the biomass.

The manufacture of a biofuel from a biomass requires accessibility to plant constituents (e.g., cellulosic materials need to be broken-down). As such, biomass may require a pre-treatment step that uses heat, chemicals and/or purified enzyme additives. These treatments are often expensive, inefficient, and produce by-products that are inhibitory of downstream processing or are toxic.

### SUMMARY

The present disclosure relates generally to methods for processing a feedstock (e.g., a biomass) by mixing the feedstock with an additive organism (e.g., a transgenic organism including but not limited to a plant, alga, or fungus) that comprises one or more transgenes coding for one or more enzymes (e.g., gene stack), including methods for processing a feedstock by preparing an additive organism that comprises one or more transgenes coding for one or more enzymes and mixing the feedstock with the additive organism. An additive organism may produce one or more enzymes from one or more transgenes and may optionally produce one or more enzymes not from transgenes.

Such methods may additionally include the step of incubating the mixture under conditions wherein the feedstock is processed by the activity of one or more enzymes on the feedstock.

The present disclosure provides methods for processing a feedstock by preparing an additive organism that comprises one or more transgenes coding for one or more enzymes (e.g., gene stack), disrupting cells of the additive organism, mixing the feedstock with the disrupted cells, and incubating the mixture under conditions wherein the feedstock is processed by the activity of the one or more enzymes on the feedstock.

The present disclosure provides methods for processing a feedstock by preparing an additive organism that comprises one or more transgenes coding for one or more enzymes (e.g., gene stack), mixing the feedstock with the additive organism, and incubating the mixture under conditions wherein the feedstock is processed by the activity of the one or more enzymes on the feedstock. In some embodiments, the additive organism can secrete one or more enzymes encoded by one or more transgenes (along with optionally other enzymes produced by the additive organism) into its environment.

The present disclosure also provides methods for converting a feedstock to a biofuel by mixing the feedstock with an additive organism that comprises one or more transgenes coding for one or more enzymes (e.g., gene stack), converting the feedstock into sugars, and fermenting or chemically converting the sugars to produce a biofuel or other hydrocarbon.

The present disclosure provides methods for converting a feedstock to a biofuel by mixing the feedstock with an additive organism that comprises one or more transgenes coding for one or more enzymes (e.g., gene stack), extracting one or more oils from the feedstock, and converting the oils to a biofuel.

The present disclosure provides method for generating revenue from a biofuel manufacturing process by mixing the feedstock with an additive organism that comprises one or more transgenes coding for one or more enzymes (e.g., gene stack), converting the feedstock into sugars and selling the sugars to a buyer (e.g., a supplier, a distributor, a manufacturer, a dealer, a reseller, a wholesaler, a retailer and/or a consumer), or fermenting or chemically converting the sugars to produce a biofuel, and selling the biofuel to a buyer (e.g., a supplier, a distributor, a manufacturer, a dealer, a reseller, a wholesaler, a retailer and/or a consumer).

The present disclosure also provides methods for generating revenue from a biofuel manufacturing process by mixing the feedstock with an additive organism that comprises one or more transgenes coding for one or more enzymes (e.g., gene stack), extracting one or more oils from the feedstock, converting the oils to a biofuel, and selling the biofuel to a buyer (e.g., a supplier, a distributor, a manufacturer, a dealer, a reseller, a wholesaler, a retailer and/or a consumer).

The present disclosure provides an additive organism for processing a feedstock, that comprises one or more transgenes coding for one or more enzymes (e.g., gene stack), wherein the enzymes include, for example, those enzymes listed in Table 1.

The present disclosure also provides methods of preparing an additive organism by introducing one or more transgenes (e.g., gene stack) into the additive organism, wherein the enzymes include, for example, those enzymes listed in Table 1.

The present disclosure also provides an additive organism for processing a feedstock, that comprises one or more transgenes (e.g., gene stack) that when transcribed produce an RNA product that is capable of inhibiting (RNAi) the production of one or more enzymes, wherein the enzymes include but are not limited to those listed in Table 1. Inhibitory RNA products include, for example, antisense RNA and microRNAs.

The present disclosure also provides methods of preparing an additive organism by introducing one or more transgenes (e.g., gene stack) into the additive organism that when transcribed produce an RNA product that is capable of inhibiting (RNAi) the production of one or more enzymes, wherein the enzymes include but are not limited to those listed in Table 1. Inhibitory RNA products include, for example, antisense RNA and microRNAs.

The present disclosure also provides methods for processing a feedstock by expressing one or more transgenes (e.g., gene stack) coding for one or more enzymes in the additive organism, and mixing a feedstock with the additive organism.

In some embodiments, the one or more transgenes are present in a nucleic acid construct that integrates into a chromosome in the additive organism. Integrative constructs include, for example, those that integrate into nuclear chromosomes, mitochondrial chromosomes, chloroplast chromosomes or any other non-nuclear portion of the genome. In some embodiments, the one or more transgenes are present in a nucleic acid construct that does not integrate into the chromosomes of the additive organism including, for example, a mini-chromosome, artificial chromosome, plasmid, episome, or synthetic chromosome. In some embodiments, the minichromosome or other nucleic acid construct further comprises an inducible promoter. In some embodiments, the promoter is induced by heat. In some embodiments, the promoter is induced by a decrease in pH. In some embodiments, the promoter is induced by an increase in pH. In some embodiments the promoter may be induced by the exogenous application of a compound. In some embodiments, the mini-chromosome or other nucleic acid construct may further comprise a tissue-specific promoter. In some embodiments, the promoter may be expressed only in the seeds. In some embodiments, the promoter may be expressed only in the leaves.

In some embodiments, the genes are introduced into the organism by direct uptake, glass bead agitation, agitation with silicon carbide or aluminum borate fibers (e.g., “whiskers”), microparticle bombardment, biologically mediated delivery (e.g., including but not limited to Agrobacterium), liposome mediated delivery or electroporation.

In some embodiments, the additive organism comprises one or more transgenes (e.g., gene stack) each of which separately comprises a polynucleotide sequence selected from the group consisting of SEQ ID NOs: 1-40 (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 and/or SEQ ID NO: 40).

In some embodiments, the additive organism comprises three transgenes (e.g., gene stack) each of which separately comprises one of the polynucleotide sequences set forth by SEQ ID NOs: 1-40. In further embodiments, the transgenes each separately comprise one of the polynucleotide sequences set forth by SEQ ID NOs: 1, 19 and 27. In further embodiments, the transgenes each separately comprise one of the polynucleotide sequences set forth by SEQ ID NOs: 15, 17 and 27.

In some embodiments, the additive organism comprises four transgenes (e.g., gene stack) each of which separately comprises one of the polynucleotide sequences set forth by SEQ ID NOs: 1-40. In further embodiments, the transgenes each separately comprise one of the polynucleotide sequences set forth by SEQ ID NOs: 7, 16, 18 and 28. In further embodiments, the transgenes each separately comprise one of the polynucleotide sequences set forth by SEQ ID NOs: 2, 16, 18 and 25.

In some embodiments, the additive organism comprises five transgenes (e.g., gene stack) each of which separately comprises one of the polynucleotide sequences set forth by SEQ ID NOs: 1-40. In further embodiments, the transgenes each separately comprise one of the polynucleotide sequences set forth by SEQ ID NOs: 3, 5, 16, 18 and 25. In further embodiments, the transgenes each separately comprise one of the polynucleotide sequences set forth by SEQ ID NOs: 6, 16, 18, 26 and 27.

In some embodiments, the additive organism comprises six transgenes (e.g., gene stack) each of which separately comprises one of the polynucleotide sequences set forth in SEQ ID NOs: 1-40. In further embodiments, the transgenes each separately comprise one of the polynucleotide sequences set forth by SEQ ID NOs: 8, 9, 10, 21, 22 and 35.

In some embodiments, the additive organism comprises 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 transgenes (e.g., gene stack) each of which separately comprises at least one and preferably more than one of the polynucleotide sequences set forth in SEQ ID NOs: 1-40 and optionally may include SEQ ID NOs: 1, 19 and 27; SEQ ID NOs: 15, 17 and 27; SEQ ID NOs: 2, 16, 18 and 25; SEQ ID NOs: 7, 16, 18 and 28; SEQ ID NOs: 6, 16, 18, 26 and 27; SEQ ID NOs: 3, 5, 16, 18 and 25; and/or SEQ ID NOs: 8, 9, 10, 21, 22 and 35.

In some embodiments, the feedstock is selected from the group consisting of: lignocellulosic material, recycled materials, forestry waste, industrial waste materials, livestock waste, and municipal wastes, oilseeds, starch-rich seeds, starch-rich plant material, algae, animal waste and vegetable oil. In some embodiments, the feedstock is genetically modified. In other embodiments, the feedstock is not genetically modified.

In some embodiments, the additive organism is added before treatment of the feedstock. In some embodiments, the additive organism is added after treatment of the feedstock. In some embodiments, the treatment comprises a thermochemical, chemical and/or biochemical component. In some embodiments, the thermo component is heat at 140° C. to 200° C. Other thermo treatment conditions contemplated by the present disclosure, include, for example, temperatures such as 210° to 220° C., 220° to 230° C., 230° to 240° C., 240° to 250° C., 250° to 260° C., 260° to 270° C., 270° to 280° C., 280° to 290° C., 290° to 300° C. or higher. In some embodiments, the chemical component is a dilute acid, including, for example, sulfuric acid.

In some embodiments, the feedstock may be pre-treated, including, for example by steam explosion, ammonia fiber explosion, acid or alkaline treatment, or physical disruption. In some embodiments, the physical disruption is by chopping, grinding, sonicating, pressing, or exposure to vacuum. In some embodiments, the physical disruption is by exposure to freezing or exposure to high temperatures. In some embodiments, the physical disruption is by the addition of chemical compounds. In some embodiments, the physical disruption is by the addition of enzymes. In some embodiments, the enzymes are cellulase, hemicellulase, pectinase, ligninase, expansins, or alpha glucosidase.

In some embodiments, the biofuel is ethanol, propanol, butanol, methanol, methane, 2,5-dimethylfuran, dimethyl ether, biodiesel (e.g., short chain acid alkyl esters), biogasoline, paraffins (e.g., alkanes) or hydrogen.

In some embodiments, the additive organism is modified to produce enzymes that result in the lysis of their own cells upon the administration of an eliciting signal. In some embodiments, the eliciting signals include exposure to specific temperatures, pH levels or exposure to chemical elicitors. In some embodiments, the additive organism is Lemna minor, Chlamdomonas reinhardii, Agaricus bisporus, Pistia Stratiotes, Dunaliella, or Chlorella.

In some embodiments, the enzyme is from a plant, protist, fungi, bacterium, archaea or animal. In some embodiments, the enzyme breaks down glucans. In some embodiments, the enzyme is selected from the group consisting of: endo-β(1,4)-glucanase, cellobiohydrolase, β-glucosidase, α/β-glucosidase, mixed-linked glucanase, endo-β(1,3)-glucanase, exo-β(1,3)-glucanase, and β-(1,6)-glucanase

In some embodiments, the enzyme breaks down xyloglucans, xylans or mannans. In some embodiments, the enzyme is selected from the group consisting of: hemi-cellulases/xylanases, endo-1,4-β-xylanases, β-xylosidases, glycosyl hydrolase, α-l-arabinofuranosidases, α-glucuronidases, xyloglucan-specific endoglucanase, oligoxyloglucan reducing end-specific xyloglucanase, α-fucosidase, α-xylosidase, endo-β(1,4)-xylanase, β-xylosidase, β-xylosidase/a-arabinosidase, acetylxylan esterase, ferulic acid esterase, α-glucuronidase, endo-β(1,4)-mannanase, β-mannosidase, and α-galactosidase.

In some embodiments, the enzyme breaks down cell wall components. In some embodiments, the enzyme is selected from the group consisting of: ligninases, acetylesterases, pectinases, pectin lyase, pectate lyase, endo-polygalacturonase, exo-polygalacturonase, pectin methyl esterase, rhamnogalacturonase, rhamnogalacturonan lyase, rhamnogalacturonan acetylesterase, α-L-rhamnosidase, endo-α(1,5)-arabinosidase, α-L-arabinofuranosidase, endo-β(1,4)-galactanase, xylogalacturonase, and β-galactosidase.

In some embodiments, the enzyme removes one or more inhibitors of fermentation. In some embodiments, the enzyme is nicotinamide adenine dinucleotide phosphate (NADPH)-dependent alcohol dehydrogenase.

In some embodiments, the enzyme improves fermentation.

In some embodiments, the enzyme produces nutrients for yeast growth. In some embodiments, the nutrients are vitamin B or lipids.

The present disclosure also provides a transgenic plant, alga or fungus (including, for example, a unicellular fungus such as a yeast) comprising one or more transgenes (e.g., gene stack) each of which separately comprise a polynucleotide selected from the group consisting of SEQ ID NOs: 1-40 (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 and/or SEQ ID NO: 40).

The present disclosure provides a transgenic plant, alga or fungus comprising three transgenes (e.g., gene stack) each of which separately comprise one of the polynucleotide sequences set forth in SEQ ID NOs: 1-40. In further embodiments, the transgenes each separately comprise one of the polynucleotide sequences set forth by SEQ ID NOs: 1, 19 and 27.

The present disclosure also provides a transgenic plant, alga or fungus comprising four transgenes (e.g., gene stack) each of which separately comprise one of the polynucleotide sequences set forth in SEQ ID NOs: 1-40. In further embodiments, the transgenes each separately comprise one of the polynucleotide sequences set forth by SEQ ID NOs: 7, 16, 18 and 28. In further embodiments, the transgenes each separately comprise one of the polynucleotide sequences set forth by SEQ ID NOs: 2, 16, 18 and 25.

The present disclosure provides a transgenic plant, alga or fungus comprising five transgenes (e.g., gene stack) each of which separately comprise one of the polynucleotide sequences set forth in SEQ ID NOs: 1-40. In further embodiments, the transgenes each separately comprise one of the polynucleotide sequences set forth by SEQ ID NOs: 3, 5, 16, 18 and 25. In further embodiments, the transgenes each separately comprise one the polynucleotide sequences set forth by SEQ ID NOs: 6, 16, 18, 26 and 27.

The present disclosure also provides a transgenic plant, alga or fungus comprising six transgenes (e.g., gene stack) each of which separately comprise one of the polynucleotide sequences set forth in SEQ ID NOs: 1-40. In further embodiments, the transgenes each separately comprise one of the polynucleotide sequences set forth by SEQ ID NOs: 8, 9, 10, 21, 22 and 35.

The present disclosure also provides a gene stack comprising one or more transgenes each of which separately comprise a polynucleotide sequence selected from the group consisting of SEQ ID NOs: 1-40.

The present disclosure also provides a multi-enzyme preparation comprising the protein products of one or more transgenes (e.g., gene stack) each of which separately comprise a polynucleotide sequence selected from the group consisting of SEQ ID NOs: 1-40 (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 and/or SEQ ID NO: 40).

The present disclosure also provides methods for degrading a feedstock to fermentable sugars, said method comprising contacting the feedstock with an effective amount of a multi-enzyme preparation derived from an additive organism, wherein one or more enzymes in the multi-enzyme preparation is encoded by a polynucleotide selected from the group consisting SEQ ID NOs: 1-40 (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 and/or SEQ ID NO: 40).

### DETAILED DESCRIPTION

The present disclosure provides methods for processing a feedstock (e.g. a biomass) by mixing the feedstock with one or more additive organisms (e.g., a transgenic organism, including, but not limited to a plant, alga or fungus) that comprises one or more transgenes coding for one or more enzymes (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 75 or more) including, for example, one more more gene stacks. Enzymes produced in an additive organism can be used to convert a feedstock to a biofuel (e.g., ethanol or biodiesel). Methods as described herein may increase the yields and/or reduce the costs of producing biofuels from feedstocks. For example, one advantage of the disclosed methods is that they allow for the production of biofuels from a diverse array of feedstocks without the need for expensive treatments that are typically needed to improve accessibility to plant constituents, including, for example, thermochemical, chemical and/or biochemical treatments. These methods can deliver enzymes that can be more active than those produced by other known methods and have the flexibility to deliver synergistic combinations of enzymes to a feedstock. The presently disclosed methods may reduce, including eliminate, costs associated with a biofuel manufacturing process by reducing, including eliminating, the need to pre-treat a feedstock, reducing, including, for example, eliminating, the costs associated with producing enzymes in microbial fermentation, reducing, including, for example, eliminating the regulatory costs associated with expressing an enzyme within a feedstock, and expanding the range of feedstock materials that are economically feasible to use.

Methods are provided for processing a feedstock by preparing an additive organism that comprises one or more transgenes coding for one or more enzymes; and mixing the feedstock with the additive organism. Optionally, the methods may include a treatment step prior to mixing the additive organism with the feedstock or after mixing the additive organism and feedstock. It is further contemplated that one or more additive organisms each comprising the same or different transgenes may be mixed with the feedstock. Optionally, the one or more transgenes may be present in a minichromosome. It is further contemplated that the additive organism(s) could be added to either a single type of feedstock or a mixture of feedstock material.

Methods are also provided for processing a feedstock by preparing an additive organism that comprises one or more transgenes coding for one or more enzymes; mixing the feedstock with the additive organism; and incubating the mixture under conditions wherein the feedstock is processed by activity of the one or more enzymes on the feedstock. Optionally, the methods may include a treatment step prior to mixing the additive organism with the feedstock or after mixing the additive organism and feedstock. It is further contemplated that one or more additive organisms each comprising the same or different transgenes may be mixed with the feedstock. Optionally, the one or more transgenes may be present in a minichromosome, a plasmid, an episome, a synthetic chromosome or they may be integrated into the genome of the additive organism.

Methods are provided for processing a feedstock by preparing an additive organism that comprises one or more transgenes coding for one or more enzymes, disrupting cells of the additive organism, mixing the feedstock with the disrupted cells, and incubating the mixture under conditions wherein the feedstock is processed by activity of the additive organism on the feedstock, including, for example, where the additive organism produces one or more enzymes from the transgene(s) or optionally produces one or more additional enzymes not from the transgene(s). Optionally, the methods may include a treatment step prior to mixing the additive organism with the feedstock or after mixing the additive organism and feedstock. It is further contemplated that one or more additive organisms each comprising the same or different transgenes may be mixed with the feedstock. Optionally, the one or more transgenes may be present in a minichromosome, a plasmid, an episome, a synthetic chromosome or they may be integrated into the genome of the additive organism.

The present disclosure provides methods for processing a feedstock by preparing an additive organism that comprises one or more transgenes coding for one or more enzymes (e.g., gene stack), mixing the feedstock with the additive organism, and incubating the mixture under conditions wherein the feedstock is processed by the activity of the one or more enzymes on the feedstock. In some embodiments, the additive organism can secrete one or more enzymes encoded by one or more transgenes (along with optionally other enzymes produced by the additive organism) into its environment.

Methods are provided for converting a feedstock to a biofuel by mixing the feedstock with an additive organism that comprises one or more transgenes coding for one or more enzymes; converting the feedstock into sugars; and fermenting the sugars to produce a biofuel. Optionally, the methods may include a treatment step prior to mixing the additive organism with the feedstock or after mixing the additive organism and feedstock. It is further contemplated that one or more additive organisms each comprising the same or different transgenes may be mixed with the feedstock. Optionally, the one or more transgenes may be present in a minichromosome, a plasmid, an episome, a synthetic chromosome or they may be integrated into the genome of the additive organism.

Methods are provided for converting a feedstock to a biofuel by mixing the feedstock with an additive organism that comprises one or more transgenes coding for one or more enzymes; extracting one or more oils from the feedstock; and converting the oils to a biofuel. Optionally, the methods may include a treatment step prior to mixing the additive organism with the feedstock or after mixing the additive organism and feedstock. It is further contemplated that one or more additive organisms each comprising the same or different transgenes may be mixed with the feedstock. Optionally, the one or more transgenes may be present in a minichromosome, a plasmid, an episome, a synthetic chromosome or they may be integrated into the genome of the additive organism.

Methods are provided for generating revenue from a biofuel manufacturing process by mixing the feedstock with an additive organism that comprises one or more transgenes coding for one or more enzymes; converting the feedstock into sugars; selling the sugar; or fermenting the sugars or chemically converting the sugars to produce a biofuel; and selling the biofuel to a buyer, including, for example, a consumer, a supplier, a distributor, a manufacturer, a dealer, a reseller, a wholesaler, a retailer or a customer. Optionally, the methods may include a treatment step prior to mixing the additive organism with the feedstock or after mixing the additive organism and feedstock. It is further contemplated that one or more additive organisms each comprising the same or different transgenes may be mixed with the feedstock. Optionally, the one or more transgenes may be present in a minichromosome, a plasmid, an episome, a synthetic chromosome or they may be integrated into the genome of the additive organism.

Methods are provided for generating revenue from a biofuel manufacturing process by mixing the feedstock with an additive organism that comprises one or more transgenes coding for one or more enzymes; extracting one or more oils from the feedstock; converting the oils to a biofuel; and selling the biofuel to a buyer. Optionally, the methods may include a treatment step prior to mixing the additive organism with the feedstock or after mixing the additive organism and feedstock. It is further contemplated that one or more additive organisms each comprising the same or different transgenes may be mixed with the feedstock. Optionally, the one or more transgenes may be present in a minichromosome, a plasmid, an episome, a synthetic chromosome or they may be integrated into the genome of the additive organism.

Methods are provided for processing a feedstock by preparing transgenic plant that comprises one or more transgenes coding for one or more enzymes; and mixing the feedstock with the transgenic plant. Optionally, the methods may include a treatment step prior to mixing the additive organism with the feedstock or after mixing the additive organism and feedstock. It is further contemplated that one or more transgenic plants each comprising the same or different transgenes may be mixed with the feedstock. It is further contemplated that a mixture of additive organisms including, for example, one or more plants and/or one or more non-plants may be used. Optionally, the one or more transgenes may be present in a minichromosome, a plasmid, an episome, a synthetic chromosome or they may be integrated into the genome of the additive organism.

Methods are also provided for processing a feedstock by preparing a transgenic plant that comprises one or more transgenes coding for one or more enzymes; mixing the feedstock with the transgenic plant; and incubating the mixture under conditions wherein the feedstock is processed by activity of the one or more enzymes on the feedstock. Optionally, the methods may include a treatment step prior to mixing the additive organism with the feedstock or after mixing the additive organism and feedstock. It is further contemplated that one or more transgenic plants each comprising the same or different transgenes may be mixed with the feedstock. It is further contemplated that a mixture of additive organisms including, for example, one or more plants and/or one or more non-plants may be used. Optionally, the one or more transgenes may be present in a minichromosome, a plasmid, an episome, a synthetic chromosome or they may be integrated into the genome of the additive organism.

Methods are provided for processing a feedstock by preparing a transgenic plant that comprises one or more transgenes coding for one or more enzymes, disrupting cells of the transgenic plant, mixing the feedstock with the disrupted cells, and incubating the mixture under conditions wherein the feedstock is processed by activity of the one or more enzymes on the feedstock. Optionally, the methods may include a treatment step prior to mixing the additive organism with the feedstock or after mixing the additive organism and feedstock. It is further contemplated that one or more transgenic plants each comprising the same or different transgenes may be mixed with the feedstock. It is further contemplated that a mixture of additive organisms including, for example, one or more plants and/or one or more non-plants may be used. Optionally, the one or more transgenes may be present in a minichromosome, a plasmid, an episome, a synthetic chromosome or they may be integrated into the genome of the additive organism.

Methods are provided for converting a feedstock to a biofuel by mixing the feedstock with a transgenic plant that comprises one or more transgenes coding for one or more enzymes; converting the feedstock into sugars; and fermenting the sugars to produce a biofuel. Optionally, the methods may include a treatment step prior to mixing the additive organism with the feedstock or after mixing the additive organism and feedstock. It is further contemplated that one or more transgenic plants each comprising the same or different transgenes may be mixed with the feedstock. It is further contemplated that a mixture of additive organisms including, for example, one or more plants and/or one or more non-plants may be used. Optionally, the one or more transgenes may be present in a minichromosome, a plasmid, an episome, a synthetic chromosome or they may be integrated into the genome of the additive organism.

Methods are provided for converting a feedstock to a biofuel by mixing the feedstock with a transgenic plant that comprises one or more transgenes coding for one or more enzymes; extracting one or more oils from the feedstock; and converting the oils to a biofuel. Optionally, the methods may include a treatment step prior to mixing the additive organism with the feedstock or after mixing the additive organism and feedstock. It is further contemplated that one or more transgenic plants each comprising the same or different transgenes may be mixed with the feedstock. It is further contemplated that a mixture of additive organisms including, for example, one or more plants and/or one or more non-plants may be used. Optionally, the one or more transgenes may be present in a minichromosome, a plasmid, an episome, a synthetic chromosome or they may be integrated into the genome of the additive organism.

Methods are provided for generating revenue from a biofuel manufacturing process by mixing the feedstock with a transgenic plant that comprises one or more transgenes coding for one or more enzymes; converting the feedstock into sugars, selling the sugars to a customer or fermenting or chemically converting the sugars to produce a biofuel; and selling the biofuel to a buyer. Optionally, the methods may include a treatment step prior to mixing the additive organism with the feedstock or after mixing the additive organism and feedstock. It is further contemplated that one or more transgenic plants each comprising the same or different transgenes may be mixed with the feedstock. It is further contemplated that a mixture of additive organisms including, for example, one or more plants and/or one or more non-plants may be used Optionally, the one or more transgenes may be present in a minichromosome, a plasmid, an episome, a synthetic chromosome or they may be integrated into the genome of the additive organism.

Methods are provided for generating revenue from a biofuel manufacturing process by mixing the feedstock with a transgenic plant that comprises one or more transgenes coding for one or more enzymes; extracting one or more oils from the feedstock; converting the oils to a biofuel; and selling the biofuel to a buyer. Optionally, the methods may include a treatment step prior to mixing the additive organism with the feedstock or after mixing the additive organism and feedstock. It is further contemplated that one or more transgenic plants each comprising the same or different transgenes may be mixed with the feedstock. It is further contemplated that a mixture of additive organisms including, for example, one or more plants and/or one or more non-plants may be used. Optionally, the one or more transgenes may be present in a minichromosome, a plasmid, an episome, a synthetic chromosome or they may be integrated into the genome of the additive organism.

Methods are provided for processing a feedstock by preparing transgenic fungus that comprises one or more transgenes coding for one or more enzymes; and mixing the feedstock with the transgenic fungus. Optionally, the methods may include a treatment step prior to mixing the additive organism with the feedstock or after mixing the additive organism and feedstock. It is further contemplated that one or more transgenic fungi each comprising the same or different transgenes may be mixed with the feedstock. It is further contemplated that a mixture of additive organisms including, for example, one or more fungi and/or one or more non-fungi may be used. Optionally, the one or more transgenes may be present in a minichromosome, a plasmid, an episome, a synthetic chromosome or they may be integrated into the genome of the additive organism.

Methods are also provided for processing a feedstock by preparing a transgenic fungus that comprises one or more transgenes coding for one or more enzymes; mixing the feedstock with the transgenic fungus; and incubating the mixture under conditions wherein the feedstock is processed by activity of the one or more enzymes on the feedstock. Optionally, the methods may include a treatment step prior to mixing the additive organism with the feedstock or after mixing the additive organism and feedstock. It is further contemplated that one or more transgenic fungi each comprising the same or different transgenes may be mixed with the feedstock. It is further contemplated that a mixture of additive organisms including, for example, one or more fungi and/or one or more non-fungi may be used. Optionally, the one or more transgenes may be present in a minichromosome, a plasmid, an episome, a synthetic chromosome or they may be integrated into the genome of the additive organism.

Methods are provided for processing a feedstock by preparing a transgenic fungus that comprises one or more transgenes coding for one or more enzymes, disrupting cells of the transgenic fungus, mixing the feedstock with the disrupted cells, and incubating the mixture under conditions wherein the feedstock is processed by activity of the one or more enzymes on the feedstock. Optionally, the methods may include a treatment step prior to mixing the additive organism with the feedstock or after mixing the additive organism and feedstock. It is further contemplated that one or more transgenic fungi each comprising the same or different transgenes may be mixed with the feedstock. It is further contemplated that a mixture of additive organisms including, for example, one or more fungi and/or one or more non-fungi may be used. Optionally, the one or more transgenes may be present in a minichromosome, a plasmid, an episome, a synthetic chromosome or they may be integrated into the genome of the additive organism.

Methods are provided for converting a feedstock to a biofuel by mixing the feedstock with a transgenic fungus that comprises one or more transgenes coding for one or more enzymes; converting the feedstock into sugars; and fermenting or chemically converting the sugars to produce a biofuel. Optionally, the methods may include a treatment step prior to mixing the additive organism with the feedstock or after mixing the additive organism and feedstock. It is further contemplated that one or more transgenic fungi each comprising the same or different transgenes may be mixed with the feedstock. It is further contemplated that a mixture of additive organisms including, for example, one or more fungi and/or one or more non-fungi may be used. Optionally, the one or more transgenes may be present in a minichromosome, a plasmid, an episome, a synthetic chromosome or they may be integrated into the genome of the additive organism.

Methods are provided for converting a feedstock to a biofuel by mixing the feedstock with a transgenic fungus that comprises one or more transgenes coding for one or more enzymes; extracting one or more oils from the feedstock; and converting the oils to a biofuel. Optionally, the methods may include a treatment step prior to mixing the additive organism with the feedstock or after mixing the additive organism and feedstock. It is further contemplated that one or more transgenic fungi each comprising the same or different transgenes may be mixed with the feedstock. It is further contemplated that a mixture of additive organisms including, for example, one or more fungi and/or one or more non-fungi may be used. Optionally, the one or more transgenes may be present in a minichromosome, a plasmid, an episome, a synthetic chromosome or they may be integrated into the genome of the additive organism.

Methods are provided for generating revenue from a biofuel manufacturing process by mixing the feedstock with a transgenic fungus that comprises one or more transgenes coding for one or more enzymes; converting the feedstock into sugars, selling the sugars to a customer; fermenting the sugars to produce a biofuel; and selling the biofuel to a buyer. Optionally, the methods may include a treatment step prior to mixing the additive organism with the feedstock or after mixing the additive organism and feedstock. It is further contemplated that one or more transgenic fungi each comprising the same or different transgenes may be mixed with the feedstock. It is further contemplated that a mixture of additive organisms including, for example, one or more fungi and/or one or more non-fungi may be used. Optionally, the one or more transgenes may be present in a minichromosome, a plasmid, an episome, a synthetic chromosome or they may be integrated into the genome of the additive organism.

Methods are provided for generating revenue from a biofuel manufacturing process by mixing the feedstock with a transgenic fungus that comprises one or more transgenes coding for one or more enzymes; extracting one or more oils from the feedstock; converting the oils to a biofuel; and selling the biofuel to a buyer. Optionally, the methods may include a treatment step prior to mixing the additive organism with the feedstock or after mixing the additive organism and feedstock. It is further contemplated that one or more transgenic fungi each comprising the same or different transgenes may be mixed with the feedstock. It is further contemplated that a mixture of additive organisms including, for example, one or more fungi and/or one or more non-fungi may be used. Optionally, the one or more transgenes may be present in a minichromosome, a plasmid, an episome, a synthetic chromosome or they may be integrated into the genome of the additive organism.

Methods are provided for processing a feedstock by preparing transgenic alga that comprises one or more transgenes coding for one or more enzymes; and mixing the feedstock with the transgenic alga. Optionally, the methods may include a treatment step prior to mixing the additive organism with the feedstock or after mixing the additive organism and feedstock. It is further contemplated that one or more transgenic alga each comprising the same or different transgenes may be mixed with the feedstock. It is further contemplated that a mixture of additive organisms including, for example, one or more alga and/or one or more non-alga may be used. Optionally, the one or more transgenes may be present in a minichromosome, a plasmid, an episome, a synthetic chromosome or they may be integrated into the genome of the additive organism.

Methods are also provided for processing a feedstock by preparing a transgenic alga that comprises one or more transgenes coding for one or more enzymes; mixing the feedstock with the transgenic alga; and incubating the mixture under conditions wherein the feedstock is processed by activity of the one or more enzymes on the feedstock. Optionally, the methods may include a treatment step prior to mixing the additive organism with the feedstock or after mixing the additive organism and feedstock. It is further contemplated that one or more transgenic alga each comprising the same or different transgenes may be mixed with the feedstock. It is further contemplated that a mixture of additive organisms including, for example, one or more alga and/or one or more non-alga may be used. Optionally, the one or more transgenes may be present in a minichromosome, a plasmid, an episome, a synthetic chromosome or they may be integrated into the genome of the additive organism.

Methods are provided for processing a feedstock by preparing a transgenic alga that comprises one or more transgenes coding for one or more enzymes, disrupting cells of the transgenic alga, mixing the feedstock with the disrupted cells, and incubating the mixture under conditions wherein the feedstock is processed by activity of the one or more enzymes on the feedstock. Optionally, the methods may include a treatment step prior to mixing the additive organism with the feedstock or after mixing the additive organism and feedstock. It is further contemplated that one or more transgenic alga each comprising the same or different transgenes may be mixed with the feedstock. It is further contemplated that a mixture of additive organisms including, for example, one or more alga and/or one or more non-alga may be used. Optionally, the one or more transgenes may be present in a minichromosome, a plasmid, an episome, a synthetic chromosome or they may be integrated into the genome of the additive organism.

Methods are provided for converting a feedstock to a biofuel by mixing the feedstock with a transgenic alga that comprises one or more transgenes coding for one or more enzymes; converting the feedstock into sugars; and fermenting the sugars to produce a biofuel. Optionally, the methods may include a treatment step prior to mixing the additive organism with the feedstock or after mixing the additive organism and feedstock. It is further contemplated that one or more transgenic alga each comprising the same or different transgenes may be mixed with the feedstock. It is further contemplated that a mixture of additive organisms including, for example, one or more alga and/or one or more non-alga may be used. Optionally, the one or more transgenes may be present in a minichromosome, a plasmid, an episome, a synthetic chromosome or they may be integrated into the genome of the additive organism.

Methods are provided for converting a feedstock to a biofuel by mixing the feedstock with a transgenic alga that comprises one or more transgenes coding for one or more enzymes; extracting one or more oils from the feedstock; and converting the oils to a biofuel. Optionally, the methods may include a treatment step prior to mixing the additive organism with the feedstock or after mixing the additive organism and feedstock. It is further contemplated that one or more transgenic alga each comprising the same or different transgenes may be mixed with the feedstock. It is further contemplated that a mixture of additive organisms including, for example, one or more alga and/or one or more non-alga may be used. Optionally, the one or more transgenes may be present in a minichromosome, a plasmid, an episome, a synthetic chromosome or they may be integrated into the genome of the additive organism.

Methods are provided for generating revenue from a biofuel manufacturing process by mixing the feedstock with a transgenic alga that comprises one or more transgenes coding for one or more enzymes; converting the feedstock into sugars; fermenting the sugars to produce a biofuel; and selling the biofuel to a buyer. Optionally, the methods may include a treatment step prior to mixing the additive organism with the feedstock or after mixing the additive organism and feedstock. It is further contemplated that one or more transgenic alga each comprising the same or different transgenes may be mixed with the feedstock. It is further contemplated that a mixture of additive organisms including, for example, one or more alga and/or one or more non-alga may be used. Optionally, the one or more transgenes may be present in a minichromosome, a plasmid, an episome, a synthetic chromosome or they may be integrated into the genome of the additive organism.

Methods are provided for generating revenue from a biofuel manufacturing process by mixing the feedstock with a transgenic alga that comprises one or more transgenes coding for one or more enzymes; extracting one or more oils from the feedstock; converting the oils to a biofuel; and selling the biofuel to a buyer. Optionally, the methods may include a treatment step prior to mixing the additive organism with the feedstock or after mixing the additive organism and feedstock. It is further contemplated that one or more transgenic alga each comprising the same or different transgenes may be mixed with the feedstock. It is further contemplated that a mixture of additive organisms including, for example, one or more alga and/or one or more non-alga may be used. Optionally, the one or more transgenes may be present in a minichromosome, a plasmid, an episome, a synthetic chromosome or they may be integrated into the genome of the additive organism.

An additive organism is provided for processing a feedstock, that comprises one or more transgenes coding for one or more enzymes, wherein the enzymes include, for example, those enzymes listed in Table 1. Methods are also provided for preparing an additive organism by introducing one or more transgenes into the additive organism, wherein the enzymes include, for example, those enzymes listed in Table 1. Optionally, the one or more transgenes may be present in a minichromosome, a plasmid, an episome, a synthetic chromosome or they may be integrated into the genome of the additive organism.

A transgenic plant is provided for processing a feedstock, that comprises one or more transgenes coding for one or more enzymes, wherein the enzymes include, for example, those enzymes listed in Table 1. Methods are also provided for preparing a transgenic plant by introducing one or more transgenes present in a minichromosome into the additive organism, wherein the enzymes include, for example, those enzymes listed in Table 1. Optionally, the one or more transgenes may be present in a minichromosome, a plasmid, an episome, a synthetic chromosome or they may be integrated into the genome of the additive organism.

In some embodiments, the one or more transgenes are present in a nucleic acid construct that integrates into the chromosomes of the additive organism. Integrative constructs include, for example, those constructs that integrate into nuclear chromosomes, mitochondrial chromosomes, chloroplast chromosomes or any other non-nuclear portion of the genome. In some embodiments, the one or more transgenes are present in a nucleic acid construct that does not integrate into the chromosomes of the additive organism including, for example, a mini-chromosome. In some embodiments, the minichromosome or other nucleic acid construct further comprises an inducible promoter. In some embodiments, the promoter is induced by heat. In some embodiments, the promoter is induced by a decrease in pH. In some embodiments, the promoter is induced by an increase in pH. In some embodiments the promoter is induced by the exogenous application of a compound. In some embodiments, the mini-chromosome or other nucleic acid construct may further comprise a tissue-specific promoter. In some embodiments, the promoter is expressed only in the seeds. In some embodiments, the promoter is expressed only in the leaves.

In some embodiments, the transgenes are introduced into the organism by direct nucleic acid uptake, glass bead agitation, agitation with silicon carbide or aluminum borate fibers (e.g., “whiskers”), biologically-mediated transformation (e.g. Agrobacterium-mediated transformation), protoplast-mediated transformation, microparticle bombardment or electroporation.

In some embodiments, the feedstock is selected from the group consisting of: lignocellulosic material, recycled materials, forestry waste, industrial waste materials, livestock waste, and municipal wastes, oilseeds, algae, animal waste and vegetable oil. In some embodiments, the feedstock is genetically modified.

In some embodiments, the methods further comprise treatment of the feedstock. In some embodiments, the treatment comprises a thermochemical, chemical and/or biochemical component. In some embodiments, the thermo component is heat at 140° C. to 200° C. In some embodiments, the biochemical component is an acid, including, for example, dilute sulfuric acid.

In some embodiments, the additive organism is added before treatment of the feedstock. In some embodiments, the additive organism is added after treatment of the feedstock. In some embodiments, the treatment is selected from the group consisting of: steam explosion, ammonia fiber explosion, acid or alkaline treatment, or physical disruption. In some embodiments, the physical disruption is by chopping, grinding, sonicating, pressing, or exposure to vacuum. In some embodiments, the physical disruption is by exposure to freezing or exposure to high temperatures. In some embodiments, the physical disruption is by the addition of chemical compounds. In some embodiments, the physical disruption is by the addition of enzymes. In some embodiments, the enzymes are cellulase, hemicellulase, pectinase, ligninase, expansins, or alpha glucosidase. In some embodiments, the additive organism is modified to produce enzymes that result in the lysis of their own cells upon the administration of an eliciting signal. In some embodiments, the eliciting signals include exposure to specific temperatures or exposure to chemical elicitors.

In some embodiments, the biofuel is ethanol, propanol, butanol, methanol, methane, 2,5-dimethylfuran, dimethyl ether, biodiesel (short chain acid alkyl esters), paraffins (alkanes), biogasoline, co-products or hydrogen.

The enzyme(s) can be from a plant, protist, fungi, bacterium, archaea or animal. In some embodiments one or more enzymes are used to convert polymers (e.g., starch and cellulose) into single sugars. In some embodiments, the enzymes are amylases and/or cellulases (e.g., endocellulase, cellobiohydrolase I and II, beta-glucosidase. In some embodiments, the enzymes are from three enzymes classes from the core of the T. reesei cellulose-degrading system (e.g., exoglucanases, endoglucanases, and β-glucosidases.

In some embodiments, the enzyme breaks down glucans. In some embodiments, the enzyme is selected from the group consisting of: endo-β(1,4)-glucanase, cellobiohydrolase, β-glucosidase, α-/β-glucosidase, mixed-linked glucanase, endo-β(1,3)-glucanase, exo-β(1,3)-glucanase, and β-(1,6)-glucanase.

In some embodiments, the enzyme breaks down xyloglucans, xylans or mannans. In some embodiments, the enzyme is selected from the group consisting of: hemi-cellulases/xylanases, endo-1,4-β-xylanases, β-xylosidases, glycosyl hydrolase, α-l-arabinofuranosidases, α-glucuronidases, xyloglucan-specific endoglucanase, oligoxyloglucan reducing end-specific xyloglucanase, a-fucosidase, a-xylosidase, endo-b(1,4)-xylanase, b-xylosidase, b-xylosidase/a-arabinosidase, acetylxylan esterase, ferulic acid esterase, α-glucuronidase, endo-b(1,4)-mannanase, b-mannosidase, and a-galactosidase.

In some embodiments, the enzyme breaks down cell wall components. In some embodiments, the enzyme is selected from the group consisting of: ligninases, acetylesterases, pectinases, pectin lyase, pectate lyase, endo-polygalacturonase, exo-polygalacturonase, pectin methyl esterase, rhamnogalacturonase, rhamnogalacturonan lyase, rhamnogalacturonan acetylesterase, a-L-rhamnosidase, endo-a(1,5)-arabinosidase, a-L-arabinofuranosidase, endo-b(1,4)-galactanase, xylogalacturonase, and b-galactosidase.

In some embodiments, the enzyme removes one or more inhibitors of fermentation. In some embodiments, the enzyme is nicotinamide adenine dinucleotide phosphate (NADPH)-dependent alcohol dehydrogenase. In some embodiments, enzymes to detoxify major inhibitors furfural and 5-hydroxymethylfurfural (HMF) include, for example, unspecified reductases and the nicotinamide adenine dinucleotide phosphate (NADPH)-dependent alcohol dehydrogenase.

In some embodiments, the enzyme is an endocellulase, including, for example, 11B E I beta-1,4-endoglucanase precursor, Acidothermus cellulolyticus (SEQ ID NO: 1); Endoglucanase I (EGI, Cel7B), Trichoderma reesei/Hypocrea jecorina (SEQ ID NO: 2); Endoglucanase II (EGII, Cel5A), Trichoderma reesei/Hypocrea jecorina (SEQ ID NO: 3); Endoglucanase II (EGII, Cel5A), Penicillium janthinellum (SEQ ID NO: 4); Endoglucanase III (Cel12A), Trichoderma longibrachiatum (SEQ ID NO: 5); Endoglucanase IV (Cel61A), Trichoderma reesei/Hypocrea jecorina (SEQ ID NO: 6); Endoglucanase V (Cel45A), Trichoderma reesei/Hypocrea jecorina (SEQ ID NO: 7); Endo-1,4-β-glucanase A (eglA), Aspergillus nidulans (SEQ ID NO: 8); Endo-1,4-β-glucanase B (eglB), Aspergillus niger (SEQ ID NO: 9); Endo-1,4-β-glucanase C (eglC), Aspergillus niger (SEQ ID NO: 10); Endo-b(1,4)-glucanase AN1602.2, Aspergillus nidulans (SEQ ID NO: 11); Endo-b(1,4)-glucanase AN5214.2, Aspergillus nidulans (SEQ ID NO: 12); Endo-b(1,4)-glucanase AN1285.2, Aspergillus nidulans (SEQ ID NO: 13); Endo-b(1,4)-glucanase AN3418.2, Aspergillus nidulans (SEQ ID NO: 14); Avicelase (AvillI), Acidothermus cellolyticus (SEQ ID NO: 15).

In some embodiments, the enzyme is an exocellulase, including, for example, Cellobiohydrolase I (CBHI), Trichoderma reesii (SEQ ID NO: 16); Cellobiohydrolase I (CBHI) (Gux1B), Neurospora crassa (SEQ ID NO: 17); Cellobiohydrolase II (CBHII), Trichoderma reesii (SEQ ID NO: 18); GuxA, Acidothermus cellolyticus (SEQ ID NO: 19); Cellobiohydrolase AN0494.2, Aspergillus nidulans (SEQ ID NO: 20); Cellobiohydrolase AN5282.2, Aspergillus nidulans (SEQ ID NO: 21); Cellobiohydrolase AN5176.2, Aspergillus nidulans (SEQ ID NO: 22).

In some embodiments, the enzyme is a beta-glucosidase, including for example, Beta-glucosidase Bgl1 (Bgl3A), Trichoderma spp (SEQ ID NO: 23); Beta-glucosidase Bgl2 (Bgl1A) Trichoderma spp (SEQ ID NO: 24); Beta-glucosidase Bgl3 Cel3b (Bgl3B), Trichoderma spp (SEQ ID NO: 25); Beta-glucosidase Bgl4 (Bgl3C), Trichoderma spp (SEQ ID NO: 26); Beta-glucosidase Bgl5 Cel1b (Bgl1B), Trichoderma spp (SEQ ID NO: 27); Beta-glucosidase Bgl6, Trichoderma spp (SEQ ID NO: 28); BGL6 Beta-Glucosidase (SEQ ID NO: 29); Beta-glucosidase Bgl7, Trichoderma spp (SEQ ID NO: 30); BGL7 Beta-glucosidase (SEQ ID NO: 31); Beta-glucosidase Cel3E (Bgl3E), Trichoderma spp (SEQ ID NO: 32); Beta-glucosidase Cel3D (Bgl3D), Trichoderma spp (SEQ ID NO: 33); b-Glucosidase AN2227.2, Aspergillus nidulans (SEQ ID NO: 34); b-Glucosidase AN2612.2, Aspergillus nidulans (SEQ ID NO: 35); b-Glucosidase AN0712.2, Aspergillus nidulans (SEQ ID NO: 36); b-Glucosidase AN1551.2, Aspergillus nidulans (SEQ ID NO: 37); b-Glucosidase AN1804.2, Aspergillus nidulans (SEQ ID NO: 38); a-/b-Glucosidase AN7345.2, Aspergillus nidulans (SEQ ID NO: 39); Beta-glucosidase, Orpinomyces (SEQ ID NO: 40).

In some embodiments, the enzyme can be used in biodiesel production. In some embodiments, the enzyme is a lipase (e.g., triacylglycerolhydrolase). In some embodiments, the enzyme is a degumming enzyme (e.g., phospholipase A, phospholipase B, phospholipase C, phospholipase D and/or patatin).

In some embodiments, the enzyme can be used to increase the value of by-products from the biofuels process. In some embodiments, the enzyme is phytase.

In some embodiments, the enzyme can be used to deodorize biofuels. In some embodiments, the enzyme is a protease, peroxidase and/or polyphenol oxidase.

In some embodiments, the enzyme improves fermentation.

In some embodiments, the enzyme produces nutrients for yeast growth. In some embodiments, the nutrients are vitamin B or lipids.

An additive organism is provided that comprises one or more transgenes (e.g., gene stack) each of which separately comprises a polynucleotide sequence selected from the group consisting of SEQ ID NOs: 1-40 (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 and/or SEQ ID NO: 40).

A transgenic plant, alga or fungus is provided that comprises one or more transgenes each of which separately comprise a polynucleotide selected from the group consisting of SEQ ID NOs: 1-40 (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 and/or SEQ ID NO: 40).

As referred to herein, the one or more transgenes may comprise a polynucleotide with a sequence that varies by one or nucleotides from the polynucleotide sequences set forth in SEQ ID NOs: 1-40, wherein those sequences encode biological equivalents (e.g., enzymatic equivalents). In some embodiments, the one or more transgenes may comprise polynucleotides that are capable of hybridizing, preferably under stringent hybridization and wash conditions, to nucleotide sequences set forth in SEQ ID NOS: 1-40.

Stringency of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

Stringent conditions or high stringency conditions, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5.times.SSC (0.75 M NaCl, 0.075 M sodium citrate), 50 mM sodium phosphate (pH 6.8), 0.1% sodium pyrophosphate, 5.times. Denhardt's solution, sonicated salmon sperm DNA (50.mu.g/ml), 0.1% SDS, and 10% dextran sulfate at 42° C., with washes at 42° C. in 0.2.times.SSC (sodium chloride/sodium citrate) and 50% formamide at 55° C., followed by a high-stringency wash consisting of 0.1.times.SSC containing EDTA at 55° C.

Moderately stringent conditions may be identified as described by Sambrooket et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5.times.SSC (150 mM NaCl, 15 mM trisodium citrate), 50 mM sodium phosphate (pH 7.6), 5.times. Denhardt's solution, 10% dextran sulfate, and 20 mg/ml denatured sheared salmon sperm DNA, followed by washing the filters in 1.times.SSC at about 37-50° C. The skilled artisan will recognize how to adjust the temperature, ionic strength, etc. as necessary to accommodate factors such as probe length and the like.

A transgenic plant, alga or fungus is also provided that comprises three transgenes each of which separately comprise one of the polynucleotide sequences set forth in SEQ ID NOs: 1-40. In further embodiments, the transgenes each separately comprise one of the polynucleotide sequences set forth by SEQ ID NOs: 1, 19 and 27.

A transgenic plant, alga or fungus is also provided that comprises four transgenes each of which separately comprise one of the polynucleotide sequences set forth in SEQ ID NOs: 1-40. In further embodiments, the transgenes each separately comprise one of the polynucleotide sequences set forth by SEQ ID NOs: 7, 16, 18 and 28. In further embodiments, the transgenes each separately comprise one of the polynucleotide sequences set forth by SEQ ID NOs: 2, 16, 18 and 25.

A transgenic plant, alga or fungus is also provided that comprises five transgenes each of which separately comprise one of the polynucleotide sequences set forth in SEQ ID NOs: 1-40. In further embodiments, the transgenes each separately comprise one of the polynucleotide sequences set forth by SEQ ID NOs: 3, 5, 16, 18 and 25. In further embodiments, the transgenes each separately comprise one the polynucleotide sequences set forth by SEQ ID NOs: 6, 16, 18, 26 and 27.

A transgenic plant, algae or fungus is also provided that comprises six transgenes each of which separately comprise one of the polynucleotide sequences set forth in SEQ ID NOs: 1-40. In further embodiments, the transgenes each separately comprise one of the polynucleotide sequences set forth by SEQ ID NOs: 8, 9, 10, 21, 22 and 35.

A transgenic plant, alga or fungus is also provided that comprises 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 transgenes each of which separately comprises at least one and preferably more than one of the polynucleotide sequences set forth in SEQ ID NOs: 1-40 and optionally may include SEQ ID NOs: 1, 19 and 27; SEQ ID NOs: 15, 17 and 27; SEQ ID NOs: 2, 16, 18 and 25; SEQ ID NOs: 7, 16, 18 and 28; SEQ ID NOs: 6, 16, 18, 26 and 27; SEQ ID NOs: 3, 5, 16, 18 and 25; and/or SEQ ID NOs: 8, 9, 10, 21, 22 and 35.

A gene stack is also provided that comprises one or more transgenes each of which separately comprise a polynucleotide sequence selected from the group consisting of SEQ ID NOs: 1-40 (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 and/or SEQ ID NO: 40).

A multi-enzyme preparation is provided that comprises the protein products of one or more transgenes each of which separately comprise a polynucleotide sequence selected from the group consisting of SEQ ID NOs: 1-40 (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 and/or SEQ ID NO: 40).

Methods are also provided for degrading a feedstock to fermentable sugars by contacting the feedstock with an effective amount of a multi-enzyme preparation derived from an additive organism, wherein one or more enzymes in the multi-enzyme preparation is encoded by a polynucleotide selected from the group consisting SEQ ID NOs: 1-40 (e.g., SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 and/or SEQ ID NO: 40).

### Construction of an Additive Organism

An additive organism may be constructed to comprise one or more transgenes. As used herein, the term “additive organism” refers to an organism that has been genetically engineered to express one or more transgenes (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 75 or more). In some embodiments, the additive organism comprises four or more transgenes. The additive organism may be a small, fast-growing organism capable of producing large quantities of recombinant protein and optionally being grown in containment. Examples of additive organisms may include small aquatic plants (e.g., Lemna), microalgae (e.g., Chiorella or Spirulina), macroalgae (e.g., Elodea), fungi (e.g. Agaricus), and protist. The additive organism can be any whole plant, plant part or organs including any part the plant (e.g., leaves, stems, roots, stalks or harvested seeds). Alternatively, the additive organism can be any whole alga, alga part or organ including, for example, any phase of the algal life cycle (e.g., zoospore, isogamete, zygote, sporophyte or gametophyte). Alternatively, the additive organism can be any whole fungus, including a unicellular fungus (e.g., a yeast), fungus part or organ including, for example, any phase of the fungal life cycle (e.g., spore, hyphae, imperfect stage, sclerotia, primordial, mycelia or fruit body).

Gene stacking can be used to produce multiple enzymes from the additive organism. Given that different feedstocks may require different combinations or ratios of specific enzymes, the additive organism may produce multiple enzymes. For example, breaking down cellulose uses a combination of at least four enzymes (one that breaks cellulose chains in their interior, two that remove pairs of sugar molecules from the ends of the sugar chains, and one that breaks the released sugar pairs into simple fermentable sugars. This can be achieved by expressing each of these enzymes in a single genetically modified additive organism. Alternatively, a mixture of two or more modified additive organisms can be used.

Gene stacking can be accomplished by several mechanisms. For example, to construct an organism with multiple transgenes, one large transgene construct can be built containing many genes. Alternatively, multiple transgenes can be introduced as separate events and combined in the same strain by crossing, or introduced by multiple transformations in series. For the former strategy, multigene constructs can be introduced as BiBACs (see, e.g., C. Hamilton, et al. (1996) PNAS USA 93: 9975-9979), or other large constructs that integrate into the host chromosome, or as engineered chromosomes, which remain autonomous from the host genome (S. Carlson, et al. (2007) PLoS Genet. 3: 1965-1974). For example, beta-carotene content has been manipulated in several species, notably Golden rice, by addition of three enzyme genes in a mini-pathway (see, e.g., X. Ye, et al. (2000) Science 287: 303-305), either by transformation with a large construct containing the three required genes, or by transformation with two independent constructs.

In some embodiments, a gene stack may comprise two or more polynucleotides encoding two or more enzymes, including polynucleotides that are one or more of SEQ ID NOs: 1-40 or biologically equivalent (e.g., enzymatically equivalent polynucleotides).

In some embodiments, a gene stack may comprise Endoglucanase I (EGI, Cel7B), Trichoderma reesei/Hypocrea jecorina (GenBank Accession Number M15665) (SEQ ID NO: 2); Cellobiohydrolase I (CBHI), Trichoderma reesii (GenBank Accession Number P62694) (SEQ ID NO: 16); Cellobiohydrolase II (CBHII), Trichoderma reesii (GenBank Accession Number M16190) (SEQ ID NO: 18); and Beta-glucosidase Bgl3 Cel3b (Bgl3B), Trichoderma spp, (GenBank Assession Number AY281374) (SEQ ID NO: 25).

In some embodiments, a gene stack may comprise Endoglucanase II (EGII, Cel5A), Trichoderma reesei/Hypocrea jecorina (GenBank Accession Number M19373) (SEQ ID NO: 3); Endoglucanase III (Cel12A), Trichoderma longibrachiatum (GenBank Accession Number AB003694) (SEQ ID NO: 5); Cellobiohydrolase 1 (CBHI), Trichoderma reesii (GenBank Accession Number P62694) (SEQ ID NO: 16); Cellobiohydrolase II (CBHII), Trichoderma reesii (GenBank Accession Number M16190) (SEQ ID NO: 18); and Beta-glucosidase Bgl3 Cel3b (Bgl3B), Trichoderma spp (GenBank Accession Number AY281374) (SEQ ID NO: 25).

In some embodiments, a gene stack may comprise Endoglucanase IV (Cel61A), Trichoderma reesei/Hypocrea jecorina (GenBank Accession Number Y11113) ((SEQ ID NO: 6); Cellobiohydrolase I (CBHI), Trichoderma reesii (GenBank Accession Number P62694) (SEQ ID NO: 16); Cellobiohydrolase II (CBHII), Trichoderma reesii (GenBank Accession Number M16190) (SEQ ID NO: 18); Beta-glucosidase Bgl4 (Bgl3C), Trichoderma spp (GenBank Accession Number AY281375) (SEQ ID NO: 26); Beta-glucosidase Bgl5 Cel1b (Bgl1B), Trichoderma spp (GenBank Accession Number AY281377) (SEQ ID NO: 27).

In some embodiments, a gene stack may comprise Endoglucanase V (Cel45A), Trichoderma reesei/Hypocrea jecorina (GenBank Accession Number Z33381) (SEQ ID NO: 7); Cellobiohydrolase I (CBHI), Trichoderma reesii (GenBank Accession Number P62694) (SEQ ID NO: 16); Cellobiohydrolase II (CBHII), Trichoderma reesii (Genbank Accession Number M16190) (SEQ ID NO: 18); and Beta-glucosidase Bgl6, Trichoderma spp (GenBank Accession Number 115264208) (SEQ ID NO: 28).

In some embodiments, a gene stack may comprise beta-1,4-endoglucanase, Acidothermus cellulolyticus (GenBank Accession Number U33212.1) (SEQ ID NO: 1); GuxA, Acidothermus cellolyticus (GenBank Accession Number AX700036) (SEQ ID NO: 19); Beta-glucosidase Bgl5 Cel1b (Bgl1B), Trichoderma spp (GenBank Accession Number AY281377) (SEQ ID NO: 27).

In some embodiments, a gene stack may comprise Avicelase (AvillI), Acidothermus cellolyticus (GenBank Accession Number AX700058) (SEQ ID NO: 15); Cellobiohydrolase I (CBHI) (Gux1B), Neurospora crassa (GenBank Accession Number X77778) (SEQ ID NO: 17); and Beta-glucosidase Bgl5 Cel1b (Bgl1B), Trichoderma spp (GenBank Accession Number AY281377) (SEQ ID NO: 27).

In some embodiments, a gene stack may comprise Endo-1,4-β-glucanase A (eglA), Aspergillus nidulans (Genbank Accession Number AB009402) (SEQ ID NO: 8); Endo-1,4-β-glucanase B (eglB), Aspergillus niger (GenBank Accession Number AJ224452) (SEQ ID NO: 9); Endo-1,4-β-glucanase C (eglC), Aspergillus niger (GenBank Accession Number AY040839) (SEQ ID NO: 10); Cellobiohydrolase, Aspergillus nidulans (GenBank Accession Number AN5282.2) (SEQ ID NO: 21); Cellobiohydrolase, Aspergillus nidulans (GenBank Accession Number AN5176.2) (SEQ ID NO: 22); b-Glucosidase, Aspergillus nidulans (GenBank Accession Number AN2612.2) (SEQ ID NO: 35).

Methods for construction of an additive organism (e.g., a transgenic plant, alga or fungus) may include the synthesis of a transformation construct, preparation of transgenic cells, and regeneration of tissue or whole organisms. Propagation of the transgenic additive organism can include, for example, sexual or asexual (vegetative) methods. Exemplary methods are detailed below.

1. Transformation Constructs

Polynucleotides coding for one or more enzymes may be introduced into a cell as a construct comprising expression control elements necessary for efficient expression. Enzymes produced in the additive organism may be modified or chosen to minimize problems with expression and undesirable agronomic effects. Expression of certain proteins can have undesirable agronomic effects on crop plants, for example, crops producing cell-wall degrading enzymes may lodge (e.g., fall over). Enzymes may be controlled by an inducible promoter which may be inactive until the additive organism is added to the biofuels process (e.g., inactive at physiological conditions, then activated by heat or pH), or sequestered by subcellular localization. Additionally, enzymes may be controlled by a tissue-specific promoter which may be active only in specific tissues (e.g. seeds or leaves).

Any enzyme known in the art is contemplated for use in the present disclosure (see, e.g., Carbohydrate Active Enzymes Database (http://www.cazy.org); P. M. Coutinho et al. (1999) in H. J. Gilbert, G. Davies, B. Henrissat and B. Svensson eds., The Royal Society of Chemistry, Cambridge, pp. 3-12; B. Henrissat (1991) Biochem. J. 280:309-316; B. Henrissat et al. (1993) Biochem. J. 293:781-788; B. Henrissat et al. (1996) Biochem. J. 316:695-696; G. Davies et al. (1995) Structure 3:853-859; B. Henrissat et al. (1997) Curr. Op. Struct. Biol. 7:637-644; J. A. Campbell et al. (1997) Biochem. J. 326:929-939; P. M. Coutinho et al. (2003) J. Mol. Biol. 328:307-317; P. M. Coutinho et al. (1999) in H. J. Gilbert, G. Davies, B. Henrissat and B. Svensson eds., The Royal Society of Chemistry, Cambridge, pp. 3-12; and A. B. Boraston et al. (2004) Biochem. J. 382:769-781. A list of exemplary enzymes is provided in Table 1.

TABLE 1
Exemplary Enzymes

| | |
|---|---|
| 1) | Glycoside Hydrolase Family 1 |
| | beta-glucosidase |
| | beta-galactosidase |
| | beta-mannosidase |
| | beta-glucuronidase |
| | beta-D-fucosidase |
| | phlorizin hydrolase |
| | 6-phospho-beta-galactosidase |
| | 6-phospho-beta-glucosidase |
| | strictosidine beta-glucosidase |
| | lactase |
| | amygdalin beta-glucosidase |
| | prunasin beta-glucosidase |
| | raucaffricine beta-glucosidase |
| | thioglucosidase |
| | beta-primeverosidase |
| | isoflavonoid 7-O-beta-apiosyl-beta-glucosidase |
| | hydroxyisourate hydrolase |
| | beta-glycosidase |
| 2) | Glycoside Hydrolase Family 2 |
| | beta-galactosidase |
| | beta-mannosidase |
| | beta-glucuronidase |
| | mannosylglycoprotein endo-beta-mannosidase |
| | exo-beta-glucosaminidase |
| 3) | Glycoside Hydrolase Family 3 |
| | beta-glucosidase |
| | xylan 1,4-beta-xylosidase |
| | beta-N-acetylhexosaminidase |
| | glucan 1,3-beta-glucosidase |
| | glucan 1,4-beta-glucosidase |
| | exo-1,3-1,4-glucanase |
| | alpha-L-arabinofuranosidase |
| 4) | Glycoside Hydrolase Family 4 |
| | maltose-6-phosphate glucosidase |
| | alpha-glucosidase |
| | alpha-galactosidase |
| | 6-phospho-beta-glucosidase |
| | alpha-glucuronidase |
| 6) | Glycoside Hydrolase Family 5 |
| | chitosanase |
| | beta-mannosidase |
| | Cellulase |
| | glucan 1,3-beta-glucosidase |
| | licheninase |
| | glucan endo-1,6-beta-glucosidase |
| | mannan endo-1,4-beta-mannosidase |
| | Endo-1,4-beta-xylanase |
| | cellulose 1,4-beta-cellobiosidase |
| | endo-1,6-beta-galactanase |
| | beta-1,3-mannanase |
| | xyloglucan-specific endo-beta-1,4-glucanase |
| 6) | Glycoside Hydrolase Family 6 |
| | endoglucanase |
| | cellobiohydrolase |
| 7) | Glycoside Hydrolase Family 7 |
| | endoglucanase |
| | reducing end-acting cellobiohydrolase |
| 8) | Glycoside Hydrolase Family 8 |
| | Chitosanase |
| | Cellulose |
| | Licheninase |
| | endo-1,4-beta-xylanase |
| | reducing-end-xylose releasing exo-oligoxylanase |
| 9) | Glycoside Hydrolase Family 9 |
| | Endoglucanase |
| | Cellobiohydrolase |
| | beta-glucosidase |
| 10) | Glycoside Hydrolase Family 10 |
| | Xylanase |
| | endo-1,3-beta-xylanase |
| 11) | Glycoside Hydrolase Family 11 |
| | Xylanase |
| 12) | Glycoside Hydrolase Family 12 |
| | endoglucanase |
| | xyloglucan hydrolase |
| | beta-1,3-1,4-glucanase |
| | xyloglucan endotransglycosylase |
| 13) | Glycoside Hydrolase Family 13 |
| | alpha-amylase |
| | pullulanase |
| | cyclomaltodextrin glucanotransferase |
| | cyclomaltodextrinase |
| | trehalose-6-phosphate hydrolase |
| | oligo-alpha-glucosidase |
| | maltogenic amylase |
| | neopullulanase |
| | alpha-glucosidase |
| | maltotetraose-forming alpha-amylase |
| | isoamylase |
| | glucodextranase |
| | maltohexaose-forming alpha-amylase |
| | branching enzyme |
| | trehalose synthase |
| | 4-alpha-glucanotransferase |
| | maltopentaose-forming alpha-amylase |
| | amylosucrase |
| | sucrose phosphorylase |
| | malto-oligosyltrehalose trehalohydrolase |
| | isomaltulose synthase |
| 14) | Glycoside Hydrolase Family 14 |
| | beta-amylase |
| 15) | Glycoside Hydrolase Family 15 |
| | Glucoamylase |
| | Glucodextranase |
| | alpha, alpha-trehalase |
| 16) | Glycoside Hydrolase Family 16 |
| | xyloglucan: xyloglucosyltransferase |
| | keratan-sulfate endo-1,4-beta-galactosidase |
| | Glucan endo-1,3-beta-D-glucosidase |
| | endo-1,3(4)-beta-glucanase |
| | Licheninase |
| | Agarase |
| | kappa-carrageenase |
| | Xyloglucanase |
| 17) | Glycoside Hydrolase Family 17 |
| | glucan endo-1,3-beta-glucosidase |
| | glucan 1,3-beta-glucosidase |
| | licheninase |
| | beta-1,3-glucan transglycosidase |
| 18) | Glycoside Hydrolase Family 18 |
| | Chitinase |
| | endo-beta-N-acetylglucosaminidase |
| 19) | Glycoside Hydrolase Family 19 |
| | Chitinase |
| 20) | Glycoside Hydrolase Family 20 |
| | beta-hexosaminidase |
| | lacto-N-biosidase |
| 21) | Glycoside Hydrolase Family 22 |
| | lysozyme type C |
| | lysozyme type i |
| | alpha-lactalbumin |
| 22) | Glycoside Hydrolase Family 23 |
| | lysozyme type G |
| | peptidoglycan lytic transglycosylase |
| 23) | Glycoside Hydrolase Family 24 |
| | Lysozyme |
| 24) | Glycoside Hydrolase Family 25 |
| | Lysozyme |
| 25) | Glycoside Hydrolase Family 26 |
| | beta-mannanase |
| | beta-1,3-xylanase |
| 26) | Glycoside Hydrolase Family 27 |
| | alpha-galactosidase |
| | alpha-N-acetylgalactosaminidase |
| | isomalto-dextranase |
| 27) | Glycoside Hydrolase Family 28 |
| | polygalacturonase |
| | exo-polygalacturonase |
| | exo-polygalacturonosidase |
| | rhamnogalacturonase |
| | endo-xylogalacturonan hydrolase |
| | rhamnogalacturonan alpha-L-rhamnopyranohydrolase |
| 28) | Glycoside Hydrolase Family 29 |
| | alpha-L-fucosidase |
| 29) | Glycoside Hydrolase Family 30 |
| | Glucosylceramidase |
| | beta-1,6-glucanase |
| | beta-xylosidase |
| 30) | Glycoside Hydrolase Family 31 |
| | alpha-glucosidase |
| | alpha-1,3-glucosidase |
| | sucrase-isomaltase |
| | alpha-xylosidase |
| | alpha-glucan lyase |
| | Isomaltosyltransferase |
| 31) | Glycoside Hydrolase Family 32 |
| | invertase |
| | inulinase |
| | 2,6-beta-fructan 6-levanbiohydrolase |
| | levanase |
| | exo-inulinase |
| | sucrose: sucrose 1-fructosyl transferase |
| | fructan fructan 1-fructosyltransferase |
| | fructan beta-(2,1)-fructosidase |
| | fructan beta-(2,6)-fructosidase |
| 32) | Glycoside Hydrolase Family 33 |
| | sialidase or neuraminidase |
| | trans-sialidase |
| 33) | Glycoside Hydrolase Family 34 |
| | sialidase or neuraminidase |
| 34) | Glycoside Hydrolase Family 35 |
| | beta-galactosidase |
| 35) | Glycoside Hydrolase Family 36 |
| | alpha-galactosidase |
| | alpha-N-acetylgalactosaminidase |
| | stachyose synthase |
| | raffinose synthase |
| 36) | Glycoside Hydrolase Family 37 |
| | alpha, alpha-trehalase |
| 37) | Glycoside Hydrolase Family 38 |
| | alpha-mannosidase |
| | alpha-mannosidase |
| 38) | Glycoside Hydrolase Family 39 |
| | alpha-L-iduronidase |
| | beta-xylosidase |
| 39) | Glycoside Hydrolase Family 42 |
| | beta-galactosidase |
| 40) | Glycoside Hydrolase Family 43 |
| | beta-xylosidase |
| | beta-1,3-xylosidase |
| | alpha-L-arabinofuranosidase |
| | arabinanase |
| | xylanase |
| | galactan 1,3-beta-galactosidase |
| 41) | Glycoside Hydrolase Family 44 |
| | endoglucanase |
| | xyloglucanase |
| 42) | Glycoside Hydrolase Family 45 |
| | Endoglucanase |
| 43) | Glycoside Hydrolase Family 46 |
| | Chitosanase |
| 44) | Glycoside Hydrolase Family 47 |
| | alpha-mannosidase |
| 45) | Glycoside Hydrolase Family 48 |
| | endoglucanase |
| | chitinase |
| | cellobiohydrolases |
| 46) | Glycoside Hydrolase Family 49 |
| | dextranase |
| | isopullulanase |
| | dextran 1,6-alpha-isomaltotriosidase |
| 47) | Glycoside Hydrolase Family 50 |
| | beta-agarase |
| 48) | Glycoside Hydrolase Family 51 |
| | alpha-L-arabinofuranosidase |
| | Endoglucanase |
| 49) | Glycoside Hydrolase Family 52 |
| | beta-xylosidase |
| 50) | Glycoside Hydrolase Family 53 |
| | endo-1,4-beta-galactanase |
| 51) | Glycoside Hydrolase Family 54 |
| | alpha-L-arabinofuranosidase |
| | beta-xylosidase |
| 52) | Glycoside Hydrolase Family 55 |
| | exo-1,3-glucanase |
| | endo-1,3-glucanase |
| 53) | Glycoside Hydrolase Family 56 |
| | Hyaluronidase |
| 54) | Glycoside Hydrolase Family 57 |
| | alpha-amylase |
| | 4-alpha-glucanotransferase |
| | alpha-galactosidase |
| | amylopullulanase |
| | branching enzyme |
| 55) | Glycoside Hydrolase Family 58 |
| | endo-N-acetylneuraminidase or endo-sialidase |
| 56) | Glycoside Hydrolase Family 59 |
| | endo-N-acetylneuraminidase or endo-sialidase |
| 57) | Glycoside Hydrolase Family 61 |
| | Endoglucanase |
| 58) | Glycoside Hydrolase Family 62 |
| | alpha-L-arabinofuranosidase |
| 59) | Glycoside Hydrolase Family 63 |
| | processing alpha-glucosidase |
| 60) | Glycoside HydrolasesFamily 64 |
| | beta-1,3-glucanase |
| 61) | Glycoside Hydrolase Family 65 |
| | trehalase |
| | maltose phosphorylase |
| | trehalose phosphorylase |
| | kojibiose phosphorylase |
| 62) | Glycoside Hydrolase Family 66 |
| | cycloisomaltooligosaccharide glucanotransferase |
| | dextranase |
| 63) | Glycoside Hydrolase Family 67 |
| | alpha-glucuronidase |
| | xylan alpha-1,2-glucuronosidase |
| 64) | Glycoside Hydrolase Family 68 |
| | levansucrase |
| | beta-fructofuranosidase |
| | Inulosucrase |
| 65) | Glycoside Hydrolase Family 70 |
| | dextransucrase |
| | alternansucrase |
| 66) | Glycoside Hydrolase Family 71 |
| | alpha-1,3-glucanase |
| 67) | Glycoside Hydrolase Family 72 |
| | beta-1,3-glucanosyltransglycosylase |
| 68) | Glycoside Hydrolase Family 73 |
| | beta-1,4-N-acetylmuramoylhydrolase |
| 69) | Glycoside Hydrolase Family 74 |
| | endoglucanase |
| | oligoxyloglucan reducing end-specific cellobiohydrolase |
| | xyloglucanase |
| | Glycoside |
| 70) | Glycoside Hydrolase Family 75 |
| | Chitosanase |
| 71) | Glycoside Hydrolase Family 76 |
| | alpha-1,6-mannanase |
| 72) | Glycoside Hydrolase Family 77 |
| | amylomaltase or 4-alpha-glucanotransferase |
| 73) | Glycoside Hydrolase Family 78 |
| | alpha-L-rhamnosidase |
| 74) | Glycoside Hydrolase Family 79 |
| | endo-beta-glucuronidase/heparanase |
| 75) | Glycoside Hydrolase Family 80 |
| | Chitosanase |
| 76) | Glycoside Hydrolase Family 81 |
| | beta-1,3-glucanase |
| 77) | Glycoside Hydrolase Family 82 |
| | iota-carrageenase |
| 78) | Glycoside Hydrolase Family 83 |
| | hemagglutinin-neuraminidase |
| 79) | Glycoside Hydrolases Family 84 |
| | N-acetyl beta-glucosaminidase |
| | Hyaluronidase |
| 80) | Glycoside Hydrolase Family 85 |
| | endo-beta-N-acetylglucosaminidase |
| 81) | Glycoside Hydrolase Family 86 |
| | beta-agarase |
| 82) | Glycoside Hydrolase Family 87 |
| | mycodextranase |
| | alpha-1,3-glucanase |
| 83) | Glycoside Hydrolase Family 88 |
| | d-4,5 unsaturated beta-glucuronyl hydrolase |
| 84) | Glycoside Hydrolase Family 89 |
| | alpha-N-acetylglucosaminidase |
| 85) | Glycoside Hydrolase Family 90 |
| | Endorhamnosidase |
| 86) | Glycoside Hydrolase Family 91 |
| | inulin fructotransferase |
| 87) | Glycoside Hydrolase Family 92 |
| | alpha-1,2-mannosidase |
| 88) | Glycoside Hydrolase Family 93 |
| | exo-1,5-alpha-L-arabinanase |
| 89) | Glycoside Hydrolases Family 94 |
| | cellobiose phosphorylase |
| | cellodextrin phosphorylase |
| | chitobiose phosphorylase |
| | cyclic beta-1,2-glucan synthase |
| 90) | Glycoside Hydrolase Family 95 |
| | alpha-1,2-L-fucosidase |
| | alpha-L-fucosidase |
| 91) | Glycoside Hydrolase Family 96 |
| | alpha-agarase |
| 92) | Glycoside Hydrolase Family 97 |
| | alpha-glucosidase |
| 93) | Glycoside Hydrolase Family 98 |
| | endo-beta-galactosidase |
| 94) | Glycoside Hydrolase Family 99 |
| | glycoprotein endo-alpha-1,2-mannosidase |
| 95) | Glycoside Hydrolase Family 100 |
| | alkaline and neutral invertase |
| 96) | Glycoside Hydrolase Family 101 |
| | endo-alpha-N-acetylgalactosaminidase |
| 97) | Glycoside Hydrolase Family 102 |
| | peptidoglycan lytic transglycosylase |
| 98) | Glycoside Hydrolase Family 103 |
| | peptidoglycan lytic transglycosylase |
| 99) | Glycoside Hydrolase Family 104 |
| | peptidoglycan lytic transglycosylase |
| 100) | Glycoside Hydrolase Family 105 |
| | unsaturated rhamnogalacturonyl hydrolase |
| 101) | Glycoside Hydrolase Family 106 |
| | alpha-L-rhamnosidase |
| 102) | Glycoside Hydrolase Family 107 |
| | sulfated fucan endo-1,4-fucanase |
| 103) | Glycoside Hydrolase Family 108 |
| | N-acetylmuramidase |
| 104) | Glycoside Hydrolase Family 109 |
| | alpha-N-acetylgalactosaminidase |
| 105) | Glycoside Hydrolase Family 110 |
| | alpha-galactosidase |
| | alpha-1,3-galactosidase |
| 106) | Glycoside Hydrolase Family 111 |
| | keratan sulfate hydrolase (endo-beta-N-acetylglucosaminidase) |
| 107) | Glycoside Hydrolase Family 112 |
| | lacto-N-biose phosphorylase or galacto-N-biose phosphorylase |
| 108) | GlycosylTransferase Family 1 |
| | UDP-glucuronosyltransferase |
| | 2-hydroxyacylsphingosine 1-beta-galactosyltransferase |
| | N-acylsphingosine galactosyltransferase |
| | flavonol 3-O-glucosyltransferase |
| | indole-3-acetate beta-glucosyltransferase |
| | sterol glucosyltransferase |
| | ecdysteroid UDP-glucosyltransferase |
| | zeaxanthin glucosyltransferase |
| | zeatin O-beta-glucosyltransferase |
| | zeatin O-beta-xylosyltransferase |
| | limonoid glucosyltransferase |
| | sinapate 1-glucosyltransferase |
| | anthocyanin 3-O-galactosyltransferase |
| | anthocyanin 5-O-glucosyltransferase |
| | anthocyanidin 3-O-glucosyltransferase |
| | dTDP-beta-2-deoxy-L-fucose alpha-L-2-deoxyfucosyltransferase |
| | UDP-beta-L-rhamnose alpha-L-rhamnosyltransferase |
| | UDP-glucose 4-hydroxybenzoate 4-O-beta-glucosyltransferase |
| | flavonol L-rhamnosyltransferase |
| 109) | GlycosylTransferase Family 2 |
| | cellulose synthase |
| | chitin synthase |
| | dolichyl-phosphate beta-D-mannosyltransferase |
| | dolichyl-phosphate beta-glucosyltransferase |
| | N-acetylglucosaminyltransferase |
| | N-acetylgalactosaminyltransferase |
| | hyaluronan synthase |
| | chitin oligosaccharide synthase |
| | beta-1,3-glucan synthase |
| | beta-1,4-mannan synthase |
| | beta-mannosylphosphodecaprenol-mannooligosaccharide alpha-1,6- |
| | mannosyltransferase |
| | alpha-1,3-L-rhamnosyltransferase |
| 110) | GlycosylTransferase Family 3 |
| | glycogen synthase |
| 111) | GlycosylTransferase Family 4 |
| | sucrose synthase |
| | sucrose-phosphate synthase |
| | alpha-glucosyltransferase |
| | lipopolysaccharide N-acetylglucosaminyltransferase |
| | GDP-Man alpha-mannosyltransferase |
| | 1,2-diacylglycerol 3-glucosyltransferase |
| | diglucosyl diacylglycerol synthase |
| | digalactosyldiacylglycerol synthase |
| | trehalose phosphorylase |
| | phosphatidylinositol alpha-mannosyltransferase |
| | UDP-Gal alpha-galactosyltransferase |
| | Xylosyltransferase |
| 112) | GlycosylTransferase Family 5 |
| | UDP-Glc: glycogen glucosyltransferase |
| | ADP-Glc: starch glucosyltransferase |
| | NDP-Glc: starch glucosyltransferase |
| | UDP-Glc: alpha-1,3-glucan synthase |
| | UDP-Glc: alpha-1,4-glucan synthase |
| 113) | GlycosylTransferase Family 6 |
| | alpha-1,3-galactosyltransferase |
| | alpha-1,3 N-acetylgalactosaminyltransferase |
| | alpha-galactosyltransferase |
| | globoside alpha-N-acetylgalactosaminyltransferase |
| 114) | GlycosylTransferase Family 7 |
| | lactose synthase |
| | beta-N-acetylglucosaminyl-glycopeptide beta-1,4-galactosyltransferase |
| | N-acetyllactosamine synthase |
| | beta-1,4-N-acetylglucosaminyltransferase |
| | xylosylprotein beta-4-galactosyltransferase |
| 115) | GlycosylTransferase Family 8 |
| | lipopolysaccharide alpha-1,3-galactosyltransferase |
| | UDP-Glc: (glucosyl) lipopolysaccharide alpha-1,2-glucosyltransferase |
| | lipopolysaccharide glucosyltransferase 1 |
| | glycogenin glucosyltransferase |
| | inositol 1-alpha-galactosyltransferase (galactinol synthase) |
| | homogalacturonan alpha-1,4-galacturonosyltransferase |
| 116) | GlycosylTransferase Family 9 |
| | lipopolysaccharide N-acetylglucosaminyltransferase |
| | heptosyltransferase |
| 117) | GlycosylTransferase Family 10 |
| | galactoside alpha-1,3/1,4-L-fucosyltransferase |
| | galactoside alpha-1,3-L-fucosyltransferase |
| | glycoprotein alpha-1,3-L-fucosyltransferase |
| 118) | GlycosylTransferase Family 11 |
| | galactoside alpha-1,2-L-fucosyltransferase |
| 119) | GlycosylTransferase Family 12 |
| | [N-acetylneuraminyl]-galactosylglucosylceramide N- |
| | acetylgalactosaminyltransferase |
| 120) | GlycosylTransferase Family 13 |
| | alpha-1,3-mannosyl-glycoprotein beta-1,2-N-acetylglucosaminyltransferase |
| 121) | GlycosylTransferase Family 14 |
| | beta-1,3-galactosyl-O-glycosyl-glycoprotein beta-1,6-N- |
| | acetylglucosaminyltransferase |
| | N-acetyllactosaminide beta-1,6-N-acetylglucosaminyltransferase |
| | protein O-beta-xylosyltransferase |
| 122) | GlycosylTransferase Family 15 |
| | glycolipid 2-alpha-mannosyltransferase |
| | GDP-mannose: alpha-1,2-mannosyltransferase |
| 123) | GlycosylTransferase Family 16 |
| | alpha-1,6-mannosyl-glycoprotein beta-1,2-N-acetylglucosaminyltransferase |
| 124) | GlycosylTransferase Family 17 |
| | beta-1,4-mannosyl-glycoprotein beta-1,4-N-acetylglucosaminyltransferase |
| 125) | GlycosylTransferase Family 18 |
| | alpha-1,3(6)-mannosylglycoprotein beta-1,6-N-acetyl-glucosaminyltransferase |
| 126) | GlycosylTransferase Family 19 |
| | lipid-A-disaccharide synthase |
| 127) | GlycosylTransferase Family 20 |
| | alpha, alpha-trehalose-phosphate synthase [UDP-forming] |
| 128) | GlycosylTransferase Family 21 |
| | UDP-glucose: ceramide beta-glucosyltransferase |
| 129) | GlycosylTransferase Family 22 |
| | dolichyl-phosphate-mannose alpha-mannosyltransferase |
| 130) | GlycosylTransferase Family 23 |
| | N-acetyl-beta-D-glucosaminide alpha-1,6-fucosyltransferase |
| 131) | GlycosylTransferase Family 24 |
| | UDP-glucose glycoprotein alpha-glucosyltransferase |
| 132) | GlycosylTransferase Family 25 |
| | lipopolysaccharide biosynthesis protein; beta-1,4-galactosyltransferase |
| | beta-1,3-glucosyltransferase |
| | beta-1,2-glucosyltransferase |
| | beta-1,2-galactosyltransferase |
| 133) | GlycosylTransferase Family 26 |
| | UDP-ManNAcA beta-N-acetyl mannosaminuronyltransferase |
| | UDP-ManNAc beta-N-acetyl-mannosaminyltransferase |
| | UDP-Glc beta-1,4-glucosyltransferase |
| 134) | GlycosylTransferase Family 27 |
| | polypeptide alpha-N-acetylgalactosaminyltransferase |
| 135) | GlycosylTransferase Family 28 |
| | 1,2-diacylglycerol 3-beta-galactosyltransferase |
| | 1,2-diacylglycerol 3-beta-glucosyltransferase |
| | Undecaprenyldiphospho-muramoylpentapeptide beta-N- |
| | acetylglucosaminyltransferase |
| 136) | GlycosylTransferase Family 29 |
| | sialyltransferase |
| | beta-galactoside alpha-2,6-sialyltransferase |
| | alpha-N-acetylgalactosaminide alpha-2,6-sialyltransferase |
| | beta-galactoside alpha-2,3-sialyltransferase |
| | N-acetyllactosaminide alpha-2,3-sialyltransferase |
| | (alpha-N-acetyl-neuraminyl-2,3-beta-galactosyl-1,3)-N-acetylgalactosaminide alpha- |
| | 2,6-sialyltransferase |
| | alpha-N-acetyl-neuraminide alpha-2,8-sialyltransferase |
| | lactosylceramide alpha-2,3-sialyltransferase |
| 137) | GlycosylTransferase Family 30 |
| | alpha-3-deoxy-D-manno-octulosonic-acid (KDO) transferase |
| 138) | GlycosylTransferase Family 31 |
| | N-acetyllactosaminide beta-1,3-N-acetylglucosaminyltransferase |
| | Glycoprotein-N-acetylgalactosamine 3-beta-galactosyltransferase |
| | fucose-specific beta-1,3-N-acetylglucosaminyltransferase |
| | globotriosylceramide beta-1,3-GalNAc transferase |
| | chondroitin synthase (beta-1,3-GlcUA and beta-1,4-GalNAc transferase |
| | chondroitin beta-1,3-glucuronyltransferase |
| | chondroitin beta-1,4-N-acetylgalactosaminyltransferase |
| 139) | GlycosylTransferase Family 32 |
| | alpha-1,6-mannosyltransferase |
| | alpha-1,4-N-acetylglucosaminyltransferase |
| | alpha-1,4-N-acetylgalactosaminyltransferase |
| 140) | GlycosylTransferase Famly 33 |
| | GDP-mannose: chitobiosyldiphosphodolichol beta-mannosyltransferase |
| 141) | GlycosylTransferase Family 34 |
| | UDP-galactose: galactomannan alpha-1,6-galactosyltransferase |
| | UDP-xylose: xyloglucan alpha-1,6-xylosyltransferase |
| | alpha-1,2-galactosyltransferase |
| 142) | GlycosylTransferase Family 35 |
| | glycogen and starch phosphorylase |
| 143) | GlycosylTransferase Family 37 |
| | galactoside 2-L-fucosyltransferase |
| 144) | GlycosylTransferase Family 38 |
| | Polysialyltransferase |
| 145) | GlycosylTransferase Family 39 |
| | Dolichyl-phosphate-mannose-protein mannosyltransferase |
| 146) | GlycosylTransferase Family 40 |
| | beta-1,3-galactofuranosyltransferases |
| 147) | GlycosylTransferase Family 41 |
| | UDP-N-acetylglucosamine: peptide N-acetylglucosaminyltransferase |
| 148) | GlycosylTransferase Family 42 |
| | alpha-2,3-sialyltransferase |
| 149) | GlycosylTransferase Family 43 |
| | beta-glucuronyltransferase |
| | UDP-Xyl: xylan beta-1,4-xylosyltransferase |
| 150) | GlycosylTransferase Family 44 |
| | UDP-glucose glucosyltransferase |
| | UDP-GlcNAc GlcNAc-transferase |
| 151) | GlycosylTransferase Family 45 |
| | alpha-GlcNAc transferase |
| 152) | GlycosylTransferase Family 46 |
| | Glycosyltransferases |
| 153) | GlycosylTransferase Family 47 |
| | heparan beta-glucuronyltransferase |
| | xyloglucan beta-galactosyltransferase |
| | heparan synthase |
| | arabinan alpha-L-arabinosyltransferase |
| 154) | GlycosylTransferase Family 48 |
| | 1,3-beta-glucan synthase |
| 155) | GlycosylTransferase Family 49 |
| | beta-1,3-N-acetylglucosaminyltransferase |
| 156) | GlycosylTransferase Family 50 |
| | Dol-P-Man alpha-1,4-marnnosyltransferase |
| 157) | GlycosylTransferase Family 51 |
| | murein polymerase |
| 158) | GlycosylTransferase Family 52 |
| | alpha-2,3-sialyltransferase |
| | alpha-glucosyltransferase |
| 159) | GlycosylTransferase Family 53 |
| | UDP-L-Ara: alpha-L-arabinosyltransferase |
| 160) | GlycosylTransferase Family 54 |
| | UDP-GlcNAc: alpha-1,3-D-mannoside beta-1,4-N-acetylglucosaminyltransferase |
| 161) | GlycosylTransferase Family 55 |
| | GDP-Man: mannosyl-3-phosphoglycerate synthase |
| 162) | GlycosylTransferase Family 56 |
| | TDP-Fuc4NAc: lipid II Fuc4NAc transferase |
| 163) | GlycosylTransferase Family 57 |
| | Dol-P-Glc alpha-1,3-glucosyltransferase |
| 164) | GlycosylTransferase Family 58 |
| | dolichol pyrophosphate-mannose alpha-1,3-mannosyltransferase |
| | dolichol pyrophosphate-Man5GlcNAc2 alpha-1,2-mannosyltransferase |
| 165) | GlycosylTransferase Family 59 |
| | Dol-P-Glc: alpha-1,2-glucosyltransferase |
| 166) | GlycosylTransferase Family 60 |
| | UDP-GlcNAc: hydroxyproline polypeptide alpha-N-acetylglucosaminyltransferase |
| 167) | GlycosylTransferase Family 61 |
| | beta-1,2-xylosyltransferase |
| 168) | GlycosylTransferase Family 62 |
| | alpha-1,2-mannosyltransferase |
| | alpha-1,6-mannosyltransferase |
| 169) | GlycosylTransferase Family 63 |
| | DNA beta-glucosyltransferase |
| 170) | GlycosylTransferase Family 64 |
| | heparan alpha-N-acetylhexosaminyltransferase |
| 171) | GlycosylTransferase Family 65 |
| | GDP-Fuc: protein O-alpha-fucosyltransferase |
| 172) | GlycosylTransferase Family 66 |
| | Oligosaccharyltransferase |
| 173) | GlycosylTransferase Family 67 |
| | phosphoglycan beta-1,3-galactosyltransferase |
| 174) | GlycosylTransferase Family 68 |
| | GDP-Fuc: protein O-alpha-fucosyltransferase |
| 175) | GlycosylTransferase Family 69 |
| | GDP-Man: alpha-1,3-mannosyltransferase |
| 176) | GlycosylTransferase Family 70 |
| | UDP-GlcA: beta-glucuronosyltransferase |
| 177) | GlycosylTransferase Family 71 |
| | alpha-mannosyltransferase |
| 178) | GlycosylTransferase Family 72 |
| | DNA alpha-glucosyltransferase |
| 179) | GlycosylTransferase Family 73 |
| | alpha-3-deoxy-D-manno-octulosonic-acid (KDO) transferase |
| 180) | GlycosylTransferase Family 74 |
| | alpha-1,2-L-fucosyltransferase |
| 181) | GlycosylTransferase Family 75 |
| | Self-glucosylating UDP-Glc beta-glucosyltransferase |
| 182) | GlycosylTransferase Family 76 |
| | Dol-P-Man: alpha-1,6-mannosyltransferase |
| 183) | GlycosylTransferase Family 77 |
| | alpha-xylosyltransferase |
| | alpha-1,3-galactosyltransferase |
| | arabinosyltransferase |
| 184) | GlycosylTransfetase Family 78 |
| | GDP-Man: alpha-mannosyltransferase (mannosylglycerate synthase) |
| 185) | GlycosylTransferase Family 79 |
| | GDP-Ara: phosphoglycan alpha-1,2-arabinopyranosyltransferase 1 |
| 186) | GlycosylTransferase Family 80 |
| | beta-galactoside alpha-2,6-sialyltransferase |
| | beta-galactoside alpha-2,3-sialyltransferase |
| 187) | GlycosylTransferase Family 81 |
| | GDP-Glc: glucosyl-3-phosphoglycerate synthase |
| 188) | GlycosylTransferase Family 82 |
| | UDP-GalNAc: beta-1,4-N-acetylgalactosaminyltransferase |
| 189) | GlycosylTransferase Family 83 |
| | undecaprenyl phosphate-L-Ara4N: 4-amino-4-deoxy-beta-L-arabinosyltransferase |
| | dodecaprenyl phosphate-beta-galacturonic acid: lipopolysaccharide core alpha- |
| | galacturonosyl transferase |
| 190) | GlycosylTransferase Family 84 |
| | cyclic beta-1,2-glucan synthase |
| 191) | GlycosylTransferase Family 85 |
| | beta-D-arabinofuranosyl monophosphoryldecaprenol: galactan alpha-D- |
| | arabinofuranosyltransferase |
| 192) | GlycosylTransferase Family 86 |
| | alpha-mannosyltransferase |
| 193) | GlycosylTransferase Family 87 |
| | polyprenol-P-Man alpha-1,2-mannosyltransferase |
| 194) | GlycosylTransferase Family 88 |
| | UDP-glucosyltransferase |
| 195) | GlycosylTransferase Family 89 |
| | beta-D-arabinofuranosyl-1-monophosphoryldecaprenol: arabinan beta-1,2- |
| | arabinofuranosyltransferase |
| 196) | GlycosylTransferase Family 90 |
| | UDP-Xyl: (mannosyl) glucuronoxylomannan/galactoxylomannan beta-1,2- |
| | xylosyltransferase |
| 197) | GlycosylTransferase Family 91 |
| | beta-1,2-mannosyltransferase |
| 198) | Polysaccharide Lyase Family 1 |
| | pectate lyase |
| | exo-pectate lyase |
| | pectin lyase |
| 199) | Polysaccharide Lyase Family 2 |
| | pectate lyase |
| | exo-polygalacturonate lyase |
| 200) | Polysaccharide Lyase Family 3 |
| | pectate lyase |
| 201) | Polysaccharide Lyase Family 4 |
| | rhamnogalacturonan lyase |
| 202) | Polysaccharide Lyase Family 5 |
| | alginate lyase |
| 203) | Polysaccharide Lyase Family 6 |
| | alginate lyase |
| | chondroitinase B |
| 204) | Polysaccharide Lyase Family 7 |
| | alginate lyase |
| | alpha-L-guluronate lyase |
| 205) | Polysaccharide Lyase Family 8 |
| | hyaluronate lyase |
| | chondroitin ABC lyase |
| | chondroitin AC lyase |
| | xanthan lyase |
| 206) | Polysaccharide Lyase Family 9 |
| | pectate lyase |
| | exopolygalacturonate lyase |
| 207) | Polysaccharide Lyase Family 10 |
| | pectate lyase |
| 208) | Polysaccharide Lyase Family 11 |
| | rhamnogalacturonan lyase |
| 209) | Polysaccharide Lyase Family 12 |
| | Heparin-sulfate lyase |
| 210) | Polysaccharide Lyase Family 13 |
| | heparin lyase |
| 211) | Polysaccharide Lyase Family 14 |
| | alginate lyase |
| | polysaccharide lyase acting on glucuronic acid |
| 212) | Polysaccharide Lyase Family 15 |
| | oligo-alginate lyase |
| 213) | Polysaccharide Lyase Family 16 |
| | hyaluronan lyase |
| 214) | Polysaccharide Lyase Family 17 |
| | alginate lyase |
| 215) | Polysaccharide Lyase Family 18 |
| | alginate lyase |
| 216) | Carbohydrate Esterase Family 1 |
| | acetyl xylan esterase |
| | cinnamoyl esterase |
| | feruloyl esterase |
| 217) | Carbohydrate Esterase Family 2 |
| | acetyl xylan esterase |
| 218) | Carbohydrate Esterase Family 3 |
| | acetyl xylan esterase |
| 219) | Carbohydrate Esterase Family 4 |
| | acetyl xylan esterase |
| | chitin deacetylase |
| | chitooligosaccharide deacetylase |
| | peptidoglycan GlcNAc deacetylase |
| | peptidoglycan N-acetylmuramic acid deacetylase |
| 220) | Carbohydrate Esterase Family 5 |
| | acetyl xylan esterase |
| | cutinase |
| 221) | Carbohydrate Esterase Family 6 |
| | acetyl xylan esterase |
| 222) | Carbohydrate Esterase Family 7 |
| | acetyl xylan esterase |
| | cephalosporin-C deacetylase |
| 223) | Carbohydrate Esterase Family 8 |
| | pectin methylesterase |
| 224) | Carbohydrate Esterase Family 9 |
| | N-acetylglucosamine 6-phosphate deacetylase |
| | N-acetylgalactosamine-6-phosphate deacetylase |
| | Carbohydrate Esterase Family 10 |
| | Arylesterase |
| | carboxyl esterase |
| | acetylcholinesterase |
| | cholinesterase |
| | sterol esterase |
| | brefeldin A esterase |
| | Carbohydrate Esterase Family 11 |
| | UDP-3-0-acyl N-acetylglucosamine deacetylase |
| | Carbohydrate Esterase Family 12 |
| | pectin acetylesterase |
| | rhamnogalacturonan acetylesterase |
| | acetyl xylan esteras |
| | Carbohydrate Esterase Family 13 |
| | pectin acetylesterase |
| | Carbohydrate Esterase Family 14 |
| | N-acetyl-1-D-myo-inosityl-2-amino-2-deoxy-alpha-D-glucopyranoside deacetylase |
| | diacetylchitobiose deacetylase |
| | Carbohydrate Esterase Family 15 |
| | 4-O-methyl-glucuronyl esterase |

Methods of operatively linking expression control elements to coding sequences, including wherein the coding sequences code for enzymes, are well known in the art (Maniatis, et al., Molecular Cloning, A Laboratory Manual, Cold Spring Harbor Press, N.Y., 1982; Sambrook et al., In: Molecular cloning: a laboratory manual, 2^{nd} Ed., Cold Spring Harbor Laboratory Press, Cold Spring Harbor, N.Y., 1989). Expression control sequences are DNA sequences involved in any way in the control of transcription or translation. Suitable expression control sequences and methods of using them are well known in the art. A promoter in particular may be used, with or without enhancer elements, 5′ untranslated region, transit or signal peptides for targeting of a protein or RNA product to a plant organelle, particularly to a chloroplast and 3′ untranslated regions such as polyadenylation sites. One skilled in the art will know that various enhancers, promoters, introns, transit peptides, targeting signal sequences, and 5′ and 3′ untranslated regions (UTRs) are useful in the design of effective plant expression vectors, such as those disclosed, for example, in U.S. Patent Application Publication 2003/01403641.

Examples of suitable promoters include, for example, those described in U.S. Pat. No. 6,437,217 (e.g., maize RS81 promoter), U.S. Pat. No. 5,641,876 (e.g., rice actin promoter), U.S. Pat. No. 6,426,446 (e.g., maize RS324 promoter), U.S. Pat. No. 6,429,362 (e.g., maize PR-1 promoter), U.S. Pat. No. 6,232,526 (e.g., maize A3 promoter), U.S. Pat. No. 6,177,611 (e.g., constitutive maize promoters), U.S. Pat. Nos. 5,322,938, 5,352,605, 5,359,142 and 5,530,196 (e.g., 35S promoter), U.S. Pat. No. 6,433,252 (e.g., maize L3 oleosin promoter), U.S. Pat. No. 6,429,357 (e.g., rice actin 2 promoter as well as a rice actin 2 intron), U.S. Pat. No. 5,837,848 (e.g., root specific promoter), U.S. Pat. No. 6,294,714 (e.g., light inducible promoters), U.S. Pat. No. 6,140,078 (e.g., salt inducible promoters), U.S. Pat. No. 6,252,138 (e.g., pathogen inducible promoters), U.S. Pat. No. 6,175,060 (e.g., phosphorus deficiency inducible promoters), U.S. Pat. No. 6,635,806 (e.g., gamma-coixin promoter), and U.S. patent application Ser. No. 09/757,089 (e.g., maize chloroplast aldolase promoter). Additional promoters that may find use are a nopaline synthase (NOS) promoter (Ebert et al., Proc. Natl. Acad. Sci. USA 84:5745-5749, 1987), the octopine synthase (OCS) promoter (which is carried on tumor-inducing plasmids of Agrobacterium tumefaciens), the caulimovirus promoters such as the cauliflower mosaic virus (CaMV) 19S promoter (Lawton et al., Plant Mol. Biol. 9:315-324, 1987), the CaMV 35S promoter (Odell et al., Nature, 313:810-812, 1985), the figwort mosaic virus 35S-promoter (Walker et al., Proc. Natl. Acad. Sci. USA, 84:6624-6628, 1987), the sucrose synthase promoter (Yang et al., Proc. Natl. Acad. Sci. USA. 87:4144-4148, 1990), the R gene complex promoter (Chandler et al., Plant Cell, 1:1175-1183, 1989), and the chlorophyll a/b binding protein gene promoter, etc. Particularly beneficial for use with the present disclosure may be CaMV35S (U.S. Pat. Nos. 5,322,938; 5,352,605; 5,359,142; and 5,530,196), FMV35S (U.S. Pat. Nos. 6,051,753; 5,378,619), a PCISV promoter (e.g. U.S. Pat. No. 5,850,019), and AGRtu.nos (GenBank Accession V00087; Depicker et al., J. Mol. Appl. Genet. 1:561, 1982; Bevan et al., NAR, 11:369, 1983) promoters.

Several promoters, including inducible promoters, are also available for expression of transgenes in fungi. These include, for example, the alcA promoter from Aspergillus nidulans (see, e.g., B. Felenbok et al. (2001) Prog. Nucleic Acid Res. Mol. Biol. 69:149-204.); the amyB promoter from Aspergillus oryzae (see, e.g., S. Tada et al. (1991) Mol. Gen. Genet. 229:301-306.); the thiA promoter from Aspergillus oryzae (see, e.g., J. Y. Shoji, et al. (2005) FEMS Microbiol Lett. 244(1):41-6); the Aspergillus amylase gene promoter (see, e.g., K. Sakaguchi et al. (1992) p. 54-99. In J. R. Kinghorn and G. Turner (ed.), Applied molecular genetics of filamentous fungi. Blackie, London, England.); the Aspergillus xylanase gene promoter (see, e.g., L. H. de Graaff et al. (1994) Mol. Microbiol. 12:479-490.); the Aspergillus arabinase gene promoter (see, e.g., M. J. Flipphi et al. (1994) Microbiology 140:2673-2682); the exIA promoter of Aspergillus awamori (see, e.g., B. C. Lokman, et al. (2003) J Biotechnol. 103(2)183-90); the cbh1 promoter of Trichoderma reesei (see, e.g., M. Ilmen, et al. Mol Gen Genet 253:303-314); the cbh2 promoter of Trichoderma reesei (see, e.g., H. Stang) et al. (1993) Curr Genet 23:115-122); the xyn1 promoter of Trichoderma reesei (see, e.g., R. L. Mach et al., (1996) Mol Microbiol 21:1273-1281); the xyn2 promoter of Trichoderma reesei (see, e.g., S Zeilinger, et al. (1996) J Biol Chem 271:25624-25629); the Agaricus bisporus glyceraldehyde-3-phosphate dehydrogenase promoter (see, e.g., T, Müller et al. (2006) Mycorrhiza. 16:437-42); the gpdll and trp2 promoters from Agaricus bisporus (see, e.g., C. Burns et al. (2006) Mol Biotechnol 32:129-38); and the Coprinopsis cinerea tub1, Lentinus edodes priA and Schizophyllum commune Sc3 promoters (see, e.g., S. Kilaru et al. (2006) Appl Microbiol Biotechnol 71:200-10). Promoters from one fungal species may be useful for expressing genes in other fungal species. Alternatively, promoters from plants, animals, algae or protists species may be useful in expressing genes in fungal species. Synthetic inducible promoters that use elements from heterologous systems have also been constructed in fungal systems and may be used to control expression of transgenes in additive fungal organisms. For example, a promoter that includes an element that binds the human estrogen receptor has been constructed in fungal systems that enables the control of gene expression by the application of estrogenic substances (see, e.g., R. Pachlinger et al. (2005) Appl Environ Microbiol. 71(2):672-8).

Several promoters, including inducible promoters, are also available for expression of transgenes in algae. These include, for example, the rbcL promoter of Chlamydomonas reinhardtii (see, e.g., K. Kato et al. (2007) J Biosci Bioeng. 104:207-13); the FOX1 gene promoter of Chlamydomonas reinhardtii (see, e.g., X. Deng et al. (2007) Eukaryot Cell. 6:2163-7); the promoter of the nitrate reductase of Dunaliella salina (see, e.g., J. Li et al. (2007) Gene 403:132-42); the promoter of the hsp70 gene of Volvox carteri (see, e.g., Q. Cheng et al. (2006) Gene 371:112-20); and the aphVlll promoters of Volvox carteri and Chlamydomonas reinhardtii (see, e.g., A, Hallmann et al. (2006) Plant Cell Rep. 25:582-91). Promoters from one algal species may be useful for expressing genes in other algal species. Alternatively, promoters from plants, animals, fungi or protists species may be useful in expressing genes in algal species. Synthetic inducible promoters that use elements from heterologous systems have also been constructed could be used to control expression of transgenes in additive algal organisms (see, e.g., R. Pachlinger et al. (2005) Appl Environ Microbiol. 71(2):672-8 and J. Gulick et al. (2005) Curr Protoc Mol Biol. Chapter 23:Unit 23.12.).

Expression of genes coding for one or more enzymes may benefit by fusion of the gene to a sequence coding for a transit peptide. Transit peptides generally refer to peptide molecules that when linked to a protein of interest directs the protein to a particular tissue, cell, subcellular location, or cell organelle. Exemplary transit peptides, include chloroplast transit peptides, mitochondrial transit peptides, nuclear targeting signals, apoplast targeting signals, endoplasmic reticulum retention signals (HDEL) and vacuolar signals.

A 5′ UTR that functions as a translation leader sequence is a DNA genetic element located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences include maize and petunia heat shock protein leaders (see, e.g., U.S. Pat. No. 5,362,865), plant virus coat protein leaders, plant rubisco leaders, among others (see, e.g., Turner and Foster, Molec. Biotechn., 3:225, 1995). In the present disclosure, 5′ UTRs that may in particular find benefit are GmHsp (see, e.g., U.S. Pat. No. 5,659,122), PhDnaK (U.S. Pat. No. 5,362,865), AtAntl, TEV (e.g., Carrington and Freed, J. Virology, 64:1590, 1990), and AGRtunos (see, e.g., GenBank Accession V00087; Bevan et al., NAR, 11:369, 1983).

The 3′ non-translated sequence, 3′ transcription termination region, or poly adenylation region means a DNA molecule linked to and located downstream of the coding region of a gene and includes polynucleotides that provide polyadenylation signal and other regulatory signals capable of affecting transcription, mRNA processing or gene expression. The polyadenylation signal functions in plants to cause the addition of polyadenylate nucleotides to the 3′ end of the mRNA precursor. The polyadenylation sequence can be derived from the natural gene, from a variety of plant genes, or from T-DNA genes. An example of a 3′ transcription termination region is the nopaline synthase 3′ region (see, e.g., nos 3′; Fraley et al. (1983) Proc. Natl. Acad. Sci. USA 80:4803,). The use of different 3′ nontranslated regions has been described (Ingelbrecht et al., Plant Cell, 1:671, 1989). Polyadenylation molecules from a Pisum sativum RbcS2 gene (see, e.g., Ps.RbcS2-E9; Coruzzi et al., EMBO J., 3:1671, 1984) and AGRtu.nos (see, e.g., Rojiyaa et al., (JP 1987201527-A), 1987, Genbank Accession E01312) may be of benefit for use with the present disclosure.

A polynucleotide molecule expression unit can be linked to a second polynucleotide molecule in an expression unit containing genetic elements for a screenable/scorable marker or for a gene conferring a desired trait. Commonly used genes for screening presumptively transformed cells include, for example, β-glucuronidase (GUS), β-galactosidase, luciferase, and chloramphenicol acetyltransferase (see, e.g., Jefferson (1987) Plant Mol. Biol. Rep., 5:387; Koncz et al., (1987)Proc. Natl. Acad. Sci., USA, 84:131; De Block et al., (1984) EMBO J., 3:1681), green fluorescent protein (GFP) (see, e.g., Chalfie et al. (1994) Science, 263:802; Haseloff et al. (1995) TIG, 11:328-329; and PCT application WO 97/41228).

An additive organism may further comprise one or more desirable characteristics associated with plant morphology, physiology, growth and development, yield, nutritional enhancement, disease or pest resistance, or environmental or chemical tolerance and may include genetic elements comprising herbicide resistance (see, e.g., U.S. Pat. Nos. 6,803,501; 6,448,476; 6,248,876; 6,225,114; 6,107,549; 5,866,775; 5,804,425; 5,633,435; 5,463,175), increased yield (see, e.g., U.S. Pat. Nos. RE38,446; 6,716,474; 6,663,906; 6,476,295; 6,441,277; 6,423,828; 6,399,330; 6,372,211; 6,235,971; 6,222,098; 5,716,837), insect control (see, e.g., U.S. Pat. Nos. 6,809,078; 6,713,063; 6,686,452; 6,657,046; 6,645,497; 6,642,030; 6,639,054; 6,620,988; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 5,959,091; 5,942,664; 5,942,658, 5,880,275; 5,763,245; 5,763,241), fungal disease resistance (see, e.g., U.S. Pat. Nos. 6,653,280; 6,573,361; 6,506,962; 6,316,407; 6,215,048; 5,516,671; 5,773,696; 6,121,436; 6,316,407; 6,506,962), virus resistance (see, e.g., U.S. Pat. Nos. 6,617,496; 6,608,241; 6,015,940; 6,013,864; 5,850,023; 5,304,730), nematode resistance (see, e.g., U.S. Pat. No. 6,228,992), bacterial disease resistance (see, e.g., U.S. Pat. No. 5,516,671), plant growth and development (see, e.g., U.S. Pat. Nos. 6,723,897; 6,518,488), starch production (see, e.g., U.S. Pat. Nos. 6,538,181; 6,538,179; 6,538,178; 5,750,876; 6,476,295), modified oils production (see, e.g., U.S. Pat. Nos. 6,444,876; 6,426,447; 6,380,462), high oil production (see, e.g., U.S. Pat. Nos. 6,495,739; 5,608,149; 6,483,008; 6,476,295), modified fatty acid content (see, e.g., U.S. Pat. Nos. 6,828,475; 6,822,141; 6,770,465; 6,706,950; 6,660,849; 6,596,538; 6,589,767; 6,537,750; 6,489,461; 6,459,018), high protein production (see, e.g., U.S. Pat. No. 6,380,466), fruit ripening (see, e.g., U.S. Pat. No. 5,512,466), enhanced animal and human nutrition (see, e.g., U.S. Pat. Nos. 6,723,837; 6,653,530; 6,5412,59; 5,985,605; 6,171,640), biopolymers (see, e.g., U.S. Pat. Nos. RE37,543; 6,228,623; 5,958,745 and U.S. Patent Publication No. US20030028917), environmental stress resistance (see, e.g., U.S. Pat. No. 6,072,103), pharmaceutical peptides and secretable peptides (see, e.g., U.S. Pat. Nos. 6,812,379; 6,774,283; 6,140,075; 6,080,560), improved processing traits (see, U.S. Pat. No. 6,476,295), improved digestibility (see, e.g., U.S. Pat. No. 6,531,648) low raffinose (see, e.g., U.S. Pat. No. 6,166,292), industrial enzyme production (see, e.g., U.S. Pat. No. 5,543,576), improved flavor (see, e.g., U.S. Pat. No. 6,011,199), nitrogen fixation (see, e.g., U.S. Pat. No. 5,229,114), hybrid seed production (see, e.g., U.S. Pat. No. 5,689,041), fiber production (see, e.g., U.S. Pat. Nos. 6,576,818; 6,271,443; 5,981,834; 5,869,720) and biofuel production (see, e.g., U.S. Pat. No. 5,998,700). Any of these or other genetic elements, methods, and transgenes may be used with the present disclosure as will be appreciated by those of skill in the art in view of the instant disclosure.

An expression unit may be provided as T-DNAs between right border (RB) and left border (LB) regions of a first plasmid together with a second plasmid carrying T-DNA transfer and integration functions in Agrobacterium. The constructs may also contain plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often Agrobacterium tumefaciens ABI, C58, or LBA4404. However, other strains known to those skilled in the art of plant transformation can function in the present disclosure.

2. Preparation of Transgenic Cells

Transforming plant cells can be achieved by any of the techniques known in the art for introduction of transgenes into cells (see, e.g., Miki et al., In: Methods in Plant Molecular Biology and Biotechnology, Glick and Thompson (Eds.) CRC Press, 67-88, 1993). Examples of such methods are believed to include virtually any method by which DNA can be introduced into a cell. Methods that have been described include electroporation as illustrated in U.S. Pat. No. 5,384,253; microprojectile bombardment as illustrated in U.S. Pat. Nos. 5,015,580; 5,550,318; 5,538,880; 6,160,208; 6,399,861; and 6,403,865; Agrobacterium-mediated transformation as illustrated in U.S. Pat. Nos. 5,635,055; 5,824,877; 5,591,616; 5,981,840; and 6,384,301; protoplast transformation as illustrated in U.S. Pat. No. 5,508,184, electroporation, chemically-assisted transformation, liposome-mediated transformation (see, e.g., A. Deshayes, et al. (1985) EMBO J. 4:2731-7.), transformation by aerosol beam (see, e.g., U.S. Pat. No. 5,240,842), carbon fiber, silicon carbide fiber or aluminum borate fiber (generally termed whiskers) (see, e.g., J. Brisibe, Exp. Bot. 51(343):187-196 (2000); Dunwell (1999) Methods Mol. Biol. 111:375-82; and U.S. Pat. No. 5,464,765), micro-injection (see, e.g., T. J. Reich, et al. (1986) Bio/Technology 4: 1001-1004) and viral-mediated transformation (see, e.g., S. B. Gelvin, (2005) Nat. Biotechnol. 23:684-5). Through the application of techniques such as these, the cells of virtually any plant species may be stably transformed and selected according to the present disclosure and these cells developed into transgenic plants. Such integrative transformation technologies can be used to target the genes encoding the desired genes into the nucleus, the chloroplast, the mitochondria or any other subcellular structure containing DNA. Alternatively, modification can be done using non-integrative technologies including the use of minichromosomes or other episomal vectors.

The most widely utilized method for introducing an expression vector into plants is based on the natural transformation system of Agrobacterium (see, for example, Horsch et al. (1985) Science, 227:1229). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant (see, e.g., Kado (1991) Crit. Rev. Plant. Sci., 10:1). Descriptions of Agrobacterium vector systems and methods for Agrobacterium-mediated gene transfer are provided by numerous references, including, for example, Moloney et al. (1989) Plant Cell Reports, 8:238; and U.S. Pat. Nos. 4,940,838 and 5,464,763. Other bacteria such as Sinorhizobium, Rhizobium, and Mesorhizobium that interact with plants naturally can be modified to mediate gene transfer to a number of diverse plants. These plant-associated symbiotic bacteria can be made competent for gene transfer by acquisition of both a disarmed Ti plasmid and a suitable binary vector (see, e.g., Brothers et al. (2005) Nature, 433:630).

Plant cells may be transformed with Agrobacterium by any method known in the art. For example, A first method may involve co-cultivation of Agrobacterium with cultured isolated protoplasts. This method may use an established culture system that allows culturing protoplasts and plant regeneration from cultured protoplasts. A second exemplary method may involve transformation of cells or tissues with Agrobacterium. This method requires (a) that the plant cells or tissues can be modified by Agrobacterium and (b) that the modified cells or tissues can be induced to regenerate into whole plants. A third exemplary method may involve transformation of seeds, apices or meristems with Agrobacterium. This method requires exposure of the meristematic cells of these tissues to Agrobacterium and micropropagation of the shoots or plan organs arising from these meristematic cells. A fourth exemplary method may involve exposing whole plants to Agrobacterium (see, e.g., Bent (2006) Methods Mol. Biol. 343: 87-103).

Procedures for growth, culture and inoculation for Agrobacterium are well known in the art. For Agrobacterium cultures, a liquid or semi-solid culture media can be used. The density of the Agrobacterium culture used for inoculation and the ratio of Agrobacterium cells to explant can vary from one system to the next, as can media, growth procedures, timing and lighting conditions.

Tranformation of dicotyledons using Agrobacterium is known in the art, and transformation of monocotyledons using Agrobacterium has been described (see, e.g., WO 94/00977; U.S. Pat. No. 5,591,616; and Negrotto et al. (2000) Plant Cell Reports 19: 798-803).

A number of wild-type and disarmed strains of Agrobacterium tumefaciens and Agrobacterium rhizogenes harboring Ti or Ri plasmids can be used for gene transfer into plants. Preferably, the Agrobacterium hosts contain disarmed Ti and Ri plasmids that do not contain the oncogenes that cause tumorigenesis or rhizogenesis. Exemplary strains include Agrobacterium tumefaciens strain C58, a nopaline-type strain that is used to mediate the transfer of DNA into a plant cell, octopine-type strains such as LBA4404 or succinamopine-type strains, e.g., EHA101 or EHA105. The use of these strains for plant transformation has been reported and the methods are familiar to those of skill in the art.

The efficiency of transformation by Agrobacterium may be enhanced by using a number of methods known in the art (see, e.g., U.S. Application No. 2004/0244075). For example, the inclusion of a natural wound response molecule such as acetosyringone (AS) to the Agrobacterium culture has been shown to enhance transformation efficiency with Agrobacterium tumefaciens (Shahla et al., (1987) Plant Molec. Biol. 8:291-298). Additionally or alternatively, transformation efficiency may be enhanced by wounding the target tissue to be modified or transformed. Wounding of plant tissue may be achieved, for example, by punching, maceration, abrasion, or bombardment with microprojectiles, (see e.g., Bidney et al., (1992) Plant Molec. Biol. 18:301-313). Additionally, the bacterial genera and tools available for gene delivery into plants that can be used to transfer genes into plants may be expanded (see, e.g., Broothaerts, et. al. (2005) Nature 433: 629-633).

Another technique that may be used to genetically transform plants involves the use of microprojectile bombardment. In an exemplary process, a nucleic acid containing the desired genetic elements to be introduced into the plant is deposited on or in small dense particles, e.g., tungsten, platinum, or preferably 1 micron gold particles, which are then delivered at a high velocity into the plant tissue or plant cells using a specialized biolistics device.

For the bombardment, cells in suspension may be concentrated on filters or solid culture medium. Alternatively, immature embryos, seedling explants, or any plant tissue or target cells may be arranged on solid culture medium. The cells to be bombarded are positioned at an appropriate distance below the microprojectile stopping plate.

Various biolistics protocols have been described that differ in the type of particle or the manner in which DNA is coated onto the particle. Any technique for coating microprojectiles that allows for delivery of transforming DNA to the target cells may be used. For example, particles may be prepared by functionalizing the surface of a gold oxide particle by providing free amine groups. DNA, having a strong negative charge, will then bind to the functionalized particles.

Parameters such as the concentration of DNA used to coat microprojectiles may influence the recovery of transformants containing a single copy of the transgene. For example, a lower concentration of DNA may not necessarily change the efficiency of the transformation but may instead increase the proportion of single copy insertion events. In this regard, ranges of approximately 1 ng to approximately 10 μg (10,000 ng), approximately 5 ng to 8 μg or approximately 20 ng, 50 ng, 100 ng, 200 ng, 500 ng, 1 μg, 2 μg, 5 μg, or 7 μg of transforming DNA may be used per each 1.0-2.0 mg of starting 1.0 micron gold particles.

Other physical and biological parameters may be varied, including, for example, manipulation of the DNA/microprojectile precipitate, factors that affect the flight and velocity of the projectiles, manipulation of the cells before and immediately after bombardment (including, for example, osmotic state, tissue hydration and the subculture stage or cell cycle of the recipient cells), the orientation of an immature embryo or other target tissue relative to the particle trajectory, and also the nature of the transforming DNA, such as linearized DNA or intact supercoiled plasmids. One may particularly wish to adjust physical parameters such as DNA concentration, gap distance, flight distance, tissue distance, and helium pressure.

The particles delivered via biolistics can be “dry” or “wet.” In the “dry” method, the mini-chromosome DNA-coated particles such as gold are applied onto a macrocarrier (e.g., a metal plate, or a carrier sheet made of a fragile material such as mylar) and dried. The gas discharge then accelerates the macrocarrier into a stopping screen, which halts the macrocarrier but allows the particles to pass through; the particles then continue their trajectory until they impact the tissue being bombarded. For the “wet” method, the droplet containing the mini-chromosome DNA-coated particles is applied to the bottom part of a filter holder, which is attached to a base which is itself attached to a rupture disk holder used to hold the rupture disk to the helium egress tube for bombardment. The gas discharge directly displaces the DNA/gold droplet from the filter holder and accelerates the particles and their DNA cargo into the tissue being bombarded. The wet biolistics method has been described in detail elsewhere but has not previously been applied in the context of plants (see, e.g., Mialhe et al. (1995) Mol Mar Biol Biotechnol. 4(4):275-83). The concentrations of the various components for coating particles and the physical parameters for delivery can be optimized using procedures known in the art.

A variety of plant cells/tissues are suitable for transformation, including, for example, immature embryos, scutellar tissue, suspension cell cultures, immature inflorescence, shoot meristem, epithelial peels, nodal explants, callus tissue, hypocotyl tissue, cotyledons, roots, and leaves, meristem cells, and gametic cells, including, for example, microspores, pollen, sperm and egg cells. It is contemplated that any cell from which a fertile plant may be regenerated may be useful as a recipient cell. Callus may be initiated from tissue sources including, for example, immature embryos, seedling apical meristems, microspore-derived embryos, roots, hypocotyls, cotyledons and the like. Those cells which are capable of proliferating as callus also are recipient cells for genetic transformation.

Any suitable plant culture medium can be used. Exemplary media include MS-based media (Murashige and Skoog (1962) Physiol. Plant, 15:473-497) or N6-based media(Chu et al. (1975) Scientia Sinica 18:659) supplemented with additional plant growth regulators including but not limited to auxins such as picloram (4-amino-3,5,6-trichloropicolinic acid), 2,4-D (2,4-dichlorophenoxyacetic acid), naphalene-acetic acid (NAA) and dicamba (3,6-dichloroanisic acid), cytokinins such as BAP (6-benzylaminopurine) and kinetin, and gibberellins. Other media additives can include, for example, amino acids, macroelements, iron, microelements, vitamins and organics, carbohydrates, undefined media components, including, for example, casein hydrolysates, an appropriate gelling agent such as a form of agar, a low melting point agarose or Gelrite if desired. Those of skill in the art are familiar with the variety of tissue culture media, which when supplemented appropriately, support plant tissue growth and development and are suitable for plant transformation and regeneration. These tissue culture media can either be purchased as a commercial preparation, or custom prepared and modified. Examples of such media include, for example, Murashige and Skoog (Mursahige and Skoog (1962) Physiol. Plant, 15:473-497), N6 (Chu et al. (1975) Scientia Sinica 18:659), Linsmaier and Skoog (Linsmaier and Skoog (1965) Physio. Plant., 18:100), Uchimiya and Murashige (Uchimiya and Murashige (1962) Plant Physiol. 15:473), Gamborg's B5 media (Gamborg et al. (1968) Exp. Cell Res. (1968) 50:151), D medium (Duncan et al. (1985) Planta, 165:322-332), McCown's Woody plant media (McCown and Lloyd (1981) HortScience 6:453), Nitsch and Nitsch (Nitsch and Nitsch (1969) Science 163:85-87), and Schenk and Hildebrandt (Schenk and Hildebrandt (1972) Can. J. Bot. 50:199-204) or derivations of these media supplemented accordingly. Those of skill in the art will appreciate that media and media supplements such as nutrients and growth regulators for use in transformation and regeneration and other culture conditions such as light intensity during incubation, pH, and incubation temperatures can be varied.

Those of skill in the art will appreciate the numerous modifications in selective regimes, media, and growth conditions that can be varied depending on the plant system and the selective agent. Typical selective agents include, for example, antibiotics such as geneticin (G418), kanamycin, paromomycin or other chemicals such as glyphosate or other herbicides. Consequently, such media and culture conditions disclosed in the present disclosure can be modified or substituted with nutritionally equivalent components, or similar processes for selection and recovery of transgenic events, and still fall within the scope of the present invention.

Many if not all of the same techniques used to deliver transgenic DNA to plants can be used for algae (see. e.g., Walker et al (2005) Plant Cell Rep. 24: 629-641). Exemplary techniques include transformation by agitation with glass beads (see, e.g., K. L. Kindle (1990) Proc Natl Acad Sci USA 87:1228-1232); particle bombardment (see, e.g., R. Debuchy et al. (1989) EMBO J. 8:2803-2809); electroporation (see, e.g., K. Shimogawara et al. (1998) Genetics 148:1821-1828); silicon-carbide whiskers (see, e.g., T. G. Dunahay (1993) Biotechniques 15:452-460); biologically mediated transformation including, for example, Agrobacterium mediated transformation (see, e.g., S. V. Kumar et al. (2004) Plant Science 166:731-738); and organellar (e.g., chloroplast, mitochondria) transformation (see, e.g., N. A. Doetsch et al. (2001) Curr Genet. 39:49-60; and B. L. Randolph-Anderson, et al. (1993) Mol Gen Genet. 236:235-244). Other exemplary techniques include aerosol-beam mediated transformation (see, e.g., U.S. Pat. No. 5,240,842), electronanospray (see, e.g., U.S. Pat. No. 6,399,362), viral mediated transformation (see, e.g., S. B. Gelvin (2005) Nat. Biotechnol. 23:684-5) and microinjection (see, e.g., T. J. Reich et al. (1986) Bio/Technology 4: 1001-1004) may be useful for algal transformation.

Many techniques are also available to transform fungi including, for example protoplast-mediated transformation (see, e.g., J. R. Fincham (1989) Microbiol Rev 53:148-70); electroporation (see, e.g., B. Ruiz-Diez (2002) J Appl Microbiol 92:189-95); particle bombardment (see, e.g., B. Ruiz-Diez (2002) J Appl Microbiol 92:189-95); biologically mediated transformation including, for example, Agrobacterium mediated transformation (see, e.g., C. B. Michielse et al. (2005) Curr Genet. 48:1-17); and liposome-mediated transformation (see, e.g., A. Poma et al. (2006) Appl Microbiol Biotechnol 72:437-41). Other techniques for fungal transformation are contemplated by the present disclosure, including, for example, aerosol-beam mediated transformation (see, e.g., U.S. Pat. No. 5,240,842), electronanospray (see, e.g., U.S. Pat. No. 6,399,362), viral mediated transformation (see, e.g., S. B. Gelvin (2005) Nat. Biotechnol. 23:684-5) and microinjection (see, e.g., T. J. et al. (1986) Bio/Technology 4: 1001-1004).

3. Plant Regeneration

Regenerating a transformed plant cell into a plant can be achieved by first culturing an explant on a shooting medium and subsequently on a rooting medium. An explant may be cultured on a callus medium before being transferred to a shooting medium. A variety of media and transfer requirements can be implemented and optimized for each plant system for plant transformation and recovery of transgenic plants. Consequently, such media and culture conditions can be modified or substituted with nutritionally equivalent components, or similar processes for selection and recovery of transgenic events.

Nutrient media may be prepared as a liquid, but this may be solidified by adding the liquid to materials capable of providing a solid support. Agar is commonly used for this purpose. Bactoagar, Hazelton agar, Gelrite, and Gelgro are specific types of solid support that are suitable for growth of plant cells in tissue culture. Some cell types will grow and divide either in liquid suspension or on solid media or on both media.

Recipient cell targets include, for example, meristem cells, callus, immature embryos and gametic cells such as microspores pollen, sperm and egg cells. Any cell from which a fertile transgenic plant may be regenerated may be used in certain embodiments. For example, immature embryos may be transformed followed by selection and initiation of callus and subsequent regeneration of fertile transgenic plants. Direct transformation of immature embryos obviates the need for long term development of recipient cell cultures. Meristematic cells (e.g., plant cells capable of continual cell division and characterized by an undifferentiated cytological appearance, normally found at growing points or tissues in plants such as root tips, stem apices, lateral buds, etc.) may also be used as a recipient plant cell. Because of their undifferentiated growth and capacity for organ differentiation and totipotency, a whole transformed plant could be recovered from a single transformed meristematic cell.

Somatic cells are of various types. Embryogenic cells are one example of somatic cells which may be induced to regenerate a plant through embryo formation. Non-embryogenic cells are those which typically will not respond in such a fashion.

Certain techniques may be used that enrich recipient cells within a cell population. For example, Type II callus development, followed by manual selection and culture of friable, embryogenic tissue, generally results in an enrichment of recipient cells for use in, for example, micro-projectile transformation.

In certain embodiments, recipient cells are selected following growth in culture. Cultured cells may be grown either on solid supports or in the form of liquid suspensions. In either instance, nutrients may be provided to the cells in the form of media, and environmental conditions controlled. There are many types of tissue culture media comprised of amino acids, salts, sugars, growth regulators and vitamins. Most of the media employed in the practice of the present disclosure will have some similar components, while the media can differ in composition and proportions of ingredients according to known tissue culture practices. For example, various cell types usually grow in more than one type of media, but will exhibit different growth rates and different morphologies, depending on the growth media. In some media, cells survive but do not divide. Media composition is also frequently optimized based on the species or cell type selected.

Various types of media suitable for culture of plant cells have been previously described. Examples of these media include, for example, the N6 medium described by Chu et al. (1975) Scientia Sinica, 18:659, and MS media (see, e.g., Murashige and Skoog, Physiol. Plant, (1962) 15:473-497). In some embodiments, it may be preferable to use a media with a somewhat lower ammonia/nitrate ratio such as N6 to promote generation of recipient cells by maintaining cells in a proembryonic state capable of sustained divisions. Woody Plant Medium (WPM) can also be used (see, for example, Lloyd and McCown (1981) Proc. Int. Plant Prop. Soc., 30:421).

The method of maintenance of cell cultures may contribute to their utility as sources of recipient cells for transformation. Manual selection of cells for transfer to fresh culture medium, frequency of transfer to fresh culture medium, composition of culture medium, and environment factors including, but not limited to, light quality and quantity and temperature are all factors in maintaining callus and/or suspension cultures that are useful as sources of recipient cells. Alternating callus between different culture conditions may be beneficial in enriching for recipient cells within a culture. For example, cells may be cultured in suspension culture, but transferred to solid medium at regular intervals. After a period of growth on solid medium, cells can be manually selected for return to liquid culture medium. Repeating this sequence of transfers to fresh culture medium may be used to enrich for recipient cells. Passing cell cultures through a 1.9 mm sieve may also be useful to maintain the friability of a callus or suspension culture and enriching for transformable cells when such cell types are used.

4. Culture and Regeneration of Transgenic Plants

Once a transgenic cell has been selected, the cell can be regenerated into a fertile transgenic plant using techniques well known in the art. The transformed plants can be subsequently analyzed to determine the presence or absence of a particular nucleic acid of interest in a DNA construct. Molecular analyses include, for example, Southern blots (see, e.g., Southern (1975) Mol. Biol. 98:503) or PCR analyses, immunodiagnostic approaches. Field evaluations can also be used. These and other well known methods can be performed to confirm the stability of the transformed plants produced by the methods disclosed. These methods are well known to those of skill in the art (see, e.g., Sambrook et al. (1989) In: Molecular cloning: a laboratory manual, 2^{nd} Ed., Cold Spring Harbor Laboratory Press, Cold Spring Harbor, N.Y.).

Transgenic plants comprising genes coding for one or more enzymes can be produced. In particular, economically important plants, including crops, trees, and other plants can be transformed with DNA constructs of the present disclosure so that they are dicamba tolerant, glyphosate tolerant or have increased tolerance. Plants that are currently considered tolerant to auxin-like herbicides may be transformed to increase their tolerance to the herbicide.

Once a transgenic plant containing a transgene is prepared, the transgene can be introduced into any plant sexually compatible with the first plant by crossing, without the need for ever directly transforming the second plant; alternatively, asexual offspring can be produced through cuttings or other vegetative cells of the transformed parent plant. As used herein the term “progeny” denotes the sexual or asexual offspring of any generation of a parent plant prepared in accordance with the present disclosure, wherein the progeny comprises a selected DNA construct prepared in accordance with the present disclosure. A “transgenic plant” may thus be of any generation. “Crossing” a plant to provide a plant line having one or more added transgenes or alleles relative to a starting plant line, as disclosed herein, is defined as the techniques that result in a particular sequence being introduced into a plant line by crossing a starting line with a donor plant line that comprises a transgene or allele of the present disclosure. To achieve this one could, for example, perform the following steps: (a) plant seeds of the first (starting line) and second (donor plant line that comprises a desired transgene or allele) parent plants; (b) grow the seeds of the first and second parent plants into plants that bear flowers; (c) pollinate a flower from the first parent plant with pollen from the second parent plant; and (d) harvest seeds produced on the first plant bearing the fertilized flower.

The present disclosure thus provides transgenic plant tissues comprising genes coding for one or more enzymes. The tissues may have been directly transformed with a gene coding for one or more enzymes or inherited the gene from a progenitor cell. Tissues provided by the present disclosure specifically include, for example, cells, embryos, immature embryos, meristematic cells, immature tassels, microspores, pollen, leaves, anthers, roots, root tips, flowers and seeds. Any such tissues, including, for example, any plant part, comprising a nucleic acid described herein, are thus provided by the present disclosure. Seeds in particular will find particular benefit for use, both for commercial or food uses in the form of grain, as well as for planting to grow additional crops.

5. Alga Regeneration

For unicellular alga, transformed cells may be cultured directly by vegetative (e.g., asexual) reproduction. For multicellular alga, such as the kelp Laminaria japonica, transformed cells can be regenerated into diploid sporophytes, but there is a long induction period for sporophyte regeneration. Alternatively, haploid gametophyte cells (male and female) can be isolated, transformed (e.g. by microparticle bombardment), and mated to form diploid sporophytes, as detailed in U.S. patent application Ser. No. 10/546,558. A variety of media and transfer requirements can be implemented and optimized for each system for algal transformation and recovery of transgenic alga. Consequently, such media and culture conditions can be modified or substituted with nutritionally equivalent components, or similar processes for selection and recovery of transgenic events.

Nutrient media is prepared as a liquid, but this may be solidified by adding the liquid to materials capable of providing a solid support. Agar is most commonly used for this purpose. Bactoagar, Hazelton agar, Gelrite, and Gelgro are specific types of solid support that are suitable for growth of algal cells in culture. Some cell types will grow and divide either in liquid suspension or on solid media or on both media.

Recipient cell targets include, for example, single cells from unicellular alga, cells and tissues from multicellular alga, sporophytes, spores, and gametophytes. Any cell from which a transgenic alga may be regenerated may be used. For example, gametophyte cells may be transformed followed by selection and fertilization, resulting in transgenic sporophytes. Direct transformation of unicellular alga may obviate the need for long term development of recipient cell cultures.

In certain embodiments, recipient cells are selected following growth in culture. Cultured cells may be grown either on solid supports or in the form of liquid suspensions. In either instance, nutrients may be provided to the cells in the form of media, and environmental conditions controlled. There are many types of algal culture media comprised of amino acids, salts, sugars, and vitamins. Most of the media employed in the practice of the present disclosure will have some similar components, while the media can differ in composition and proportions of ingredients according to known culture practices. For example, various cell types usually grow in more than one type of media, but will exhibit different growth rates and different morphologies, depending on the growth media. In some media, cells survive but do not divide. Media composition is also frequently optimized based on the species or cell type selected. Various types of media suitable for culture of algal cells have been previously described. Examples of these media include seawater, ESAW medium (see, e.g., P. J. Harrison et al. (1980). J. Phycol. 16, 28-35.), AK medium (see, e.g., M. D. Keller et al. (1987). J. Phycol. 23, 633-638.), Walne medium and the Guillard's F/₂ medium (see, e.g., Laboratory techniques for the cultivation of microalgae A Vonshak—Handbook of Microalgal Mass Culture, 1986—CRC Press).

The method of maintenance of cell cultures may contribute to their utility as sources of recipient cells for transformation. Manual selection of cells for transfer to fresh culture medium, frequency of transfer to fresh culture medium, composition of culture medium, and environment factors including, for example, light quality and quantity, medium circulation, carbon dioxide content, and temperature are all factors in maintaining cells and/or suspension cultures that are useful as sources of recipient cells.

6. Culture and Regeneration of Transgenic Alga

Once a transgenic cell is selected, alga can be propagated in culture. The transformed alga can be subsequently analyzed to determine the presence or absence of a particular nucleic acid of interest in a DNA construct. Molecular analyses can include, for example, Southern blots (see, e.g., Southern, (1975) Mol. Biol. 98:503) or PCR analyses, immunodiagnostic approaches. Evaluations in large-scale culture can also be used. These and other well known methods can be performed to confirm the stability of the transformed alga produced by the methods disclosed. These methods are known to those of skill in the art (see, e.g., Sambrook et al., In: Molecular cloning: a laboratory manual, 2^{nd} Ed., Cold Spring Harbor Laboratory Press, Cold Spring Harbor, N.Y., 1989).

Transgenic alga comprising genes coding for one or more enzymes can be produced. In particular, economically important alga, including, for example, Kelps (e.g., Brown algae), Laminaria, Macrocystis, Diatoms, Chlorophyta and other micro- and macroalgae can be transformed with DNA constructs of the present disclosure so that they are resistant to the antibiotics chloramphenicol or hygromycin, or to the herbicide Basta.

Once a transgenic alga containing a transgene is prepared, the transgene can be introduced into any alga sexually compatible with the first alga by crossing, without the need for directly transforming the second alga. Therefore, as used herein the term “progeny” denotes the offspring of any generation of a parent alga prepared in accordance with the present disclosure, wherein the progeny comprises a selected DNA construct prepared in accordance with the present disclosure. “Transgenic alga” may thus be of any generation. “Crossing” alga to provide a line having one or more added transgenes or alleles relative to a starting algal line, as disclosed herein, is defined as the techniques that result in a particular sequence being introduced into an algal line by crossing a starting line with a donor algal line that comprises a transgene or allele of the present disclosure. To achieve this one could, for example, perform the following steps: (a) grow cells of opposite mating type, (b) induce gametogenesis (for example, by nitrogen starvation) (c) mix the cells and plate onto agar plates incubate for several days, and (d) isolate diploid progeny cells. Gamete isolation and mating protocol will vary for different species.

The present disclosure thus provides transgenic algal cells and tissues comprising genes coding for one or more enzymes. The tissues may have been directly transformed with a gene coding for one or more enzymes or inherited the gene from a progenitor cell. Tissues provided by the present disclosure specifically include, for example, cells, spores, gametes, and multicellular tissues. Any such tissues, including, for example, any algal part, comprising a nucleic acid described herein, are thus provided by the present disclosure. Cells of unicellular alga in particular will find particular benefit for use, both for commercial or food uses in the form of nutritional supplements and animal feed, as well as for propagation to grow additional cultures.

7. Fungi Regeneration

For unicellular fungi such as yeasts, including, for example, Saccharomyces cerevisiae, and Schizosaccharomyces pombe, transformed cells can be cultured directly by vegetative (asexual) reproduction, either as haploid cells or as diploid cells. For multicellular fungi, such as the basidiomycete Agaricus bisporus, and ascomycetes of the genera Aspergillus and Trichoderma, transgenic vegetative cells (e.g., from fruiting body cultures) cells can be regenerated and propagated as vegetative cells. These cells can also be induced to produce fruiting bodies for sexual reproduction (see, e.g., X. Chen, et al., (2000) Appl Environ Microbiol 66: 4510-4513).

A variety of media and transfer requirements can be implemented and optimized for each system for fungal transformation and recovery of transgenic fungi. Consequently, such media and culture conditions can be modified or substituted with nutritionally equivalent components, or similar processes for selection and recovery of transgenic events.

Nutrient media is often prepared as a liquid, but this may be solidified by adding the liquid to materials capable of providing a solid support. Agar is commonly used for this purpose. Bactoagar, Hazelton agar, Gelrite, and Gelgro are specific types of solid support that are suitable for growth of fungal cells in culture. Additionally, some growth media for multicellular fungi such as Agaricus bisporus, are solid, such as whole grains, compost, and peat. Some cell types will grow and divide either in liquid suspension or on solid media or on both media.

Recipient cell targets include, for example, diploid or haploid single cells from unicellular fungi, and cells and tissues from multicellular fungi, including, for example, fruiting body cells, basidiospores, and hyphal cells. Any cell from which a transgenic fungus may be regenerated may be used in certain embodiments. For example, fruiting body cells may be transformed followed by selection resulting in transgenic fungi. Direct transformation of unicellular fungi may obviate the need for long-term development of recipient cell cultures.

In certain embodiments, recipient cells are selected following growth in culture. Cultured cells may be grown either on solid supports or in the form of liquid suspensions. In either instance, nutrients may be provided to the cells in the form of media, and environmental conditions controlled. There are many types of fungal culture media comprised of amino acids, salts, sugars, and vitamins. Most of the media employed in the practice of the present disclosure will have some similar components, while the media can differ in composition and proportions of ingredients according to known culture practices. For example, various cell types usually grow in more than one type of media, but will exhibit different growth rates and different morphologies, depending on the growth media. In some media, cells survive but do not divide. Media composition is also frequently optimized based on the species or cell type selected. Various types of media suitable for culture of fungal cells have been described. Examples of these media include plant extracts such as fruit juices (e.g., for winemaking), grain extracts containing fermentable sugars (e.g., wort for beer-making), sugar-containing extracts of biomass (e.g., starch, cellulose, or other feedstock that has been broken down by enzymes into fermentable sugars), and various defined media including YPD (e.g., 1% yeast extract, 1% peptone, 2% glucose), YES (e.g., 0.5% yeast extract, 3% glucose), cornmeal dextrose agar, and potato starch dextrose agar.

The method of maintenance of cell cultures may contribute to their utility as sources of recipient cells for transformation. Manual selection of cells for transfer to fresh culture medium, frequency of transfer to fresh culture medium, composition of culture medium, and environment factors including, but not limited to, temperature, humidity, and light, are all factors in maintaining cells and/or suspension cultures that are useful as sources of recipient cells.

8. Culture and Regeneration of Transgenic Fungi

Once a transgenic cell is selected, fungi can be propagated in culture. The transformed fungi can be subsequently analyzed to determine the presence or absence of a particular nucleic acid of interest in a DNA construct. Molecular analyses can include, for example, Southern blots (see, e.g., Southern (1975) Mol. Biol. 98:503,) or PCR analyses, immunodiagnostic approaches. Evaluations in large-scale culture can also be used. These and other well known methods can be performed to confirm the stability of the transformed fungi produced by the methods disclosed. These methods are known to those of skill in the art (see, e.g., Sambrook et al., In: Molecular cloning: a laboratory manual, 2^{nd} Ed., Cold Spring Harbor Laboratory Press, Cold Spring Harbor, N.Y., 1989).

Transgenic fungi comprising genes coding for one or more enzymes can thus be produced. In particular, economically important fungi, including, for example, Saccharomyces, Agaricus, Aspergillus, and Trichoderma can be transformed with DNA constructs of the present disclosure so that they are resistant to the antibiotic hygromycin.

Once a transgenic fungus containing a transgene is prepared, that transgene can be introduced into any fungus sexually compatible with the first fungus by crossing, without the need for directly transforming the second fungus. Therefore, as used herein the term “progeny” denotes the offspring of any generation of a parent fungus prepared in accordance with the present disclosure, wherein the progeny comprises a selected DNA construct prepared in accordance with the present disclosure. “Transgenic fungi” may thus be of any generation. “Crossing” fungi to provide a line having one or more added transgenes or alleles relative to a starting fungal line, as disclosed herein, is defined as the techniques that result in a particular sequence being introduced into an fungal line by crossing a starting line with a donor fungal line that comprises a transgene or allele of the present disclosure. To achieve this one could, for example, perform the following steps: (a) grow cells of opposite mating type, (b) induce gametogenesis (c) mix the cells and plate onto agar plates incubate for several days, and (d) isolate diploid progeny cells. Gamete isolation and mating protocol will vary for different species.

The present disclosure thus provides transgenic fungal cells and tissues comprising genes coding for one or more enzymes. The tissues may have been directly transformed with a gene coding for one or more enzymes or inherited the gene from a progenitor cell. Tissues provided by the present disclosure specifically include, for example, cells, spores, and multicellular tissues. Any such tissues, including, for example, any fungal part, comprising a nucleic acid described herein, are thus provided by the present disclosure. Cells of microbial fungi in particular will find particular benefit for use, both for commercial or food uses in the form of nutritional supplements and animal feed, as well as for propagation to grow additional cultures.

### Methods for Processing a Feedstock

The present disclosure provides methods for processing a feedstock by mixing the feedstock with one or more additive organisms that comprise one or more transgenes coding for one or more enzymes. The additive organism may be added to one or more feedstocks where the expression of one or more enzymes in the additive organism processes or facilitates the processing of the feedstock. The additive organism can be any whole plant, fungus, alga or protist; plant part or organ, fungus part or organ, alga part or organ or protist part or organ, including from any phase of the plant, fungus, alga or protist life cycle.

An additive organism with a gene stack coding for enzymes optimized to a specific process (e.g., converting polymers to sugars), capable of being grown in large quantities in containment, can be engineered to produce large quantities of enzymes at low cost since the costly purification of enzymes is avoided by adding the enzyme-containing additive organism directly to a feedstock, and since the costs of producing enzymes from microbes grown in sterile sugar-rich media can be reduced or eliminated. Expression of these enzymes may be constrained to avoid problems associated with temporal and spatial expression.

It is contemplated that related species, varieties, hybrids and cultivars of the following feedstocks may be used in the disclosed methods. Such feedstocks may be genetically modified or not genetically modified.

Feedstocks contemplated by the present disclosure include, but are not limited to: sugar and starch crops, including, for example, sugar cane (e.g., Saccharum spp.), sugar beet (e.g., Beta vulgaris), sweet sorghum (e.g., Sorghum spp.), grain sorghum (e.g., Sorghum spp.), maize (e.g., Zea mays), wheat (e.g., Triticum spp.), rye (e.g., Secale cereale), barley (e.g., Hordeum vulgare), oats (e.g., Avena sativa), cassaya (e.g., Manihot esculenta), white potato (e.g., Solanum tuberosum), sweet potato (e.g., Ipomoea batatas), rice (e.g., Oryza spp.) and nypa palm (e.g., Nypa fruticans).

Feedstocks may also include oil crops, including, for example, maize (e.g., Zea mays), oil palm (e.g., Elaeis guineensis and e.g., Elaeis oleifera), soybean (e.g., Glycine max), peanut (e.g., Arachis hypogaea), cotton (e.g., Gossypium spp.), sunflower (e.g., Helianthus spp.), rapeseed (e.g., Brassica napus), olive (e.g., Olea europaea), hazelnut (e.g., Corylus avellana), linseed oil (e.g., Linum usitatissimum), safflower (e.g., Carthamus tinctorius), castor bean (e.g., Ricinus communis), coconut (e.g., Cocos Nucifera), false flax (e.g., Camelina sativa), hemp (e.g., Cannabis sativa), ramtil (e.g., Guizotia oleifera), tung (e.g., Aleurites fordii), copaifera (e.g., Copaifera langsdorfii), jojoba (e.g., Simmondsia chinensis), milk bush (e.g., Euphorbia tirucalli), karanj plant (e.g., Pongamia pinnata), neem (e.g., Azadirachta indica), petroleum nut (e.g., Pittosporum resiniferum), jatropha (e.g., Jatropha curcas), radish (e.g., Raphanus sativus), rice (e.g., Oryza spp.), honge oil (e.g., Pongamia pinnata), cashew nut (e.g., Anacardium occidentale), oats (e.g., Avena sativa), lupine (e.g., Lupinus spp.), kenaf (e.g., Hibiscus cannabinus), calendula (e.g., Calendula officinalis), coffee (e.g., Coffea arabica), euphorbia (e.g., Euphorbia antisyphilytica), pumpkin seed (e.g., Cucurbita pepo), coriander (e.g., Coriandrum sativum), sesame (e.g., Sesamum indicum), cocoa (e.g., Theobroma cacao), poppy (e.g., Papaver spp.), pecan nuts (e.g., Carya illinoinensis), macadamia nuts (e.g., Macademia spp.), brazil nuts (e.g., Bertholletia excelsa), avocado (e.g., Persea Americana) and chinese tallow (e.g., Triadica sebifera).

Feedstocks may also include alga, including, for example, microalgae, diatoms, cyanobacteria and macroalgae (e.g., seaweed). More specifically, the following are examples of possible algal feedstocks: dinoflagellates, including, for example, Crypthecodinium cohnii; thraustochytrids, including, for example, Thraustochytrium spp., Schizochytrium spp., and Ulkenia spp.; diatoms, including, for example, (e.g., Bacillariophyceae): Achnanthes spp., Amphora spp., Caloneis spp., Camphylodiscus spp., Cymbella spp., Entomoneis spp., Gyrosigma spp., Melosira spp., Fragilaria spp., Cylindrotheca spp., Navicula spp., Nitzschia spp., Pleurosigma spp., Surirella spp., Chaetoceros muelleri, Cyclotella spp., and Phaeodactylum tricornutum; green algae (Chlorophyceae), including, for example, Chlamydomonas spp., Chlorella spp., Scenedesmus spp., Ankistrodesmus spp., Chlorococcum spp., Monoraphidium minutum, Nannochloris spp., Oocystis spp., Neochloris oleoabundans, Dunaliella primolecta, Botryococcus braunii, Tetraselmis suecica; blue-green algae (cyanobacteria or Cyanophyceae), including, for example, Synechococcus spp., Oscillatoria spp.; golden algae (Chrysophyceae), including, for example, Boekelovia spp., Isochrysis spp.; Prymnesiophyceae and Eustigmatophyceae, including, for example, Nannochloropsis spp.

Feedstocks may also include trees, including, for example, short rotation plantations (e.g. willow (Salix spp.), poplar (e.g., Populus sp.) and eucalyptus (e.g., Eucalyptus sp.)).

Feedstocks may also include grasses, including, for example, switchgrass (e.g., Panicum virgatum), Miscanthus (e.g., Miscanthus spp.), reed canarygrass (e.g., Phalaris arundinacea), giant reed (e.g., Arundo Donax), bermudagrass (e.g., Cynodon dactylon) and napiergrass (e.g., Pennisetum purpureum).

Feedstocks may also include agricultural residues, including, for example, corn stover, sugarcane bagasse, sugarcane leaves, straw, prunings from vineyards and fruit trees, citrus peels, whey, waste oils, including, for example, lard, beef tallow, used frying oils and yellow grease.

Feedstocks may also include forestry waste, including, for example, logging residues, salvageable dead wood and wood chips from thinnings.

Feedstocks may also include municipal waste (e.g., solid waste), including, for example, paper, yard, food wastes, and other organic non-fossil-fuel derived materials such as textiles, natural rubber, leather found in the waste streams of urban areas, and sewage sludge.

Feedstocks may also include industrial waste, including, for example, waste wood, sawdust from sawmills, bark, chunks, slabs, shavings, and sawdust, fibrous vegetable waste from paper industries and black liquor.

Feedstocks may also include animal waste or livestock waste, including, for example, solid, dry manure to be directly combusted: beef cattle manure produced in feedlots, dairy cattle manure produced in drylots, poultry manures; wet manures handled as slurries: swine manure and dairy cattle manure produced in enclosed confinement operations.

Feedstocks may also include contaminated waste, including, for example, construction and demolition wood, lawn and tree trimmings, site-clearing wastes, wood pallets and wood packaging.

In some embodiments, the feedstock is selected from the group consisting of: lignocellulosic material, recycled materials, forestry waste, industrial waste materials, livestock waste, and municipal wastes, oilseeds, starch-rich seeds, starch-rich plant material, algae, animal waste and vegetable oil.

In some embodiments, the feedstock is genetically modified.

In other embodiments, the feedstock is not genetically modified.

It is contemplated that related species, varieties, hybrids and cultivars of the following additive organisms may be used in the disclosed methods.

Additive organisms contemplated by the present disclosure include, but are not limited to the following. Exemplary Classes include but are not limited to: Arthoniomycetes, Chaetothyriomycetes, Chytridiomycetes, Dothideomycetes, Euascomycetes, Euholobasidiomycetes, Eurotiomycetes, Glomeromycetes, Heterobasidiomycetes, Homobasidiomycetes, Laboulbeniomycetes, Lecanoromycetes, Leotiomycetes, Lichinomycetes, Neolectomycetes, Orbiliomycetes, Pezizomycetes, Pneumocystidomycetes, Protoascomycetes, Saccharomycetes, Schizosaccharomycetes, Sordariomycetes, Taphrinomycetes, Trichomycetes, Urediniomycetes, Ustilaginomycetes, Ustomycetes and Zygomycetes. Example Orders include but are not limited to: Acarosporales, Agaricales, Agaricostilbales, Agyriales, Amoebidiales, Aphyllophorales, Arachnomycetales, Archaeosporales, Arthoniales, Asellariales, Atractiellales, Auriculariales, Blastocladiales, Boletales, Boliniales, Calosphaeriales, Cantharellales, Capnodiales, Ceratobasidiales, Chaetosphaeriales, Chaetothyriales, Christianseniales, Chytridiales, Classiculales, Coniochaetales, Coronophorales, Coryneliales, Cryptomycocolacales, Cystobasidiales, Cystofilobasidiales, Dacrymycetales, Diaporthales, Dimargaritales, Diversisporales, Doassansiales, Dothideales, Eccrinales, Endogonales, Entomophthorales, Entorrhizales, Entylomatales, Eurotiales, Exobasidiales, Filobasidiales, Gautieriales, Geastrales, Georgefischeriales, Glomales, Gyalectales, Halosphaeriales, Harpellales, Helotiales, Hericiales, Heterogastridiales, Hymenochaetales, Hymenogastrales, Hypocreales, Hysteriales, Jahnulales, Kickxellales, Lecanorales, Leucosporidiales, Lichinales, Lulworthiales, Lycoperdales, Malasseziales, Medeolariales, Meliolales, Microascales, Microbotryales, Microstromatales, Monoblepharidales, Mortierellales, Mucorales, Mycocaliciales, Myriangiales, Neocallimastigales, Neolectales, Nidulariales, Onygenales, Ophiostomatales, Orbiliales, Ostropales, Paraglomales, Patellariales, Peltigerales, Pertusariales, Pezizales, Phallales, Phyllachorales, Platygloeales, Pleosporales, Pneumocystidales, Protomycetales, Pyrenulales, Rhizophydialies, Saccharomycetales, Schizosaccharomycetales, Sebacinales, Septobasidiales, Sirodesmium, Sordariales, Spathulosporales, Spizellomycetales, Sporidiobolales, Stereales, Taphrinales, Thelephorales, Tilletiales, Tremellales, Trichosphaeriales, Trichosporonales, Trichotheliales, Tulasnellales, Tulostomatales, Uredinales, Urocystales, Ustilaginales, Verrucariales, Xylariales and Zoopagales.

Monocotyledonous and dicotyledonous plants. For example the family Lemnaceae: there are four known genera and 34 species of duckweed as follows: genus Lemna (L. aequinoctialis, L. disperma, L. ecuadoriensis, L. gibba, L. japonica, L. minor, L. miniscula, L. obscura, L. perpusilla, L. tenera, L. trisulca, L. turionifera, L. valdiviana); genus Spirodela (S. intermedia, S. polyrrhiza, S. punctata); genus Wolffia (Wa. angusta, Wa. arrhiza, Wa. australina, Wa. borealis, Wa. brasiliensis, Wa. columbiana, Wa. elongata, Wa. globosa, Wa. microscopica, Wa. neglecta) and genus Wolfiella (WI. caudata, WI. denticulata, WI. gladiata, WI. hyalina, WI. lingulata, WI. repunda, WI. rotunda, and WI. neotropica). Any other genera or species of Lemnaceae, if they exist, are also aspects of the present invention. Lemna gibba, Lemna minor, and Lemna miniscula are preferred, with Lemna minor and Lemna miniscula being most preferred. Lemna species can be classified using the taxonomic scheme described by Landolt, Biosystematic Investigation on the Family of Duckweeds: The family of Lemnaceae—A Monograph Study. Geobatanischen Institut ETH, Stiftung Rubel, Zurich (1986)). Other examples of plants include, for example, Pistia Stratiotes and Medicago truncatula.

Alga including but not limited to those belonging to the orders Chlorophyta, Chlorokybales, Klebsormidiales, Zygnematales, Desmidiales, Coleochaetales and Charales (stoneworts).

Fungi including, but not limited to, those belonging to the phyla Chytridiomycota, Blastocladiomycota, Neocallimastigomycota, Zygomycota, Glomeromycota, Dikarya, Ascomycota, and Basidiomycota. Example species include but are not limited to: Agaricus bisporus, Aspergillus niger, Aspergillus terreus, Aspergillus oryzae, Aspergillus phoenicis, Trichoderma viride, Trichoderma reesei, Trichoderma konignii, Rhizopus delemar, Trametes versicolor, Mucor miehei, Sclerotium rolfsii, Aureobasidium pollulans, Schizophyllum commune, Acremonium chrysogenum, Tolypocladium nivenum, Tolypocladium inflatum, Claviceps purpurea, Monascus rubber, Taxomyces andrenae, Fusarium graminearum and Mucor cirinelloides.

The additive organism can be added directly to the feedstock. Depending on the stability of the enzymes to the conditions of biomass, the additive organism could be added before or after treatment of the feedstock.

Depending on how easily the additive organism cells can be lysed, they could be added without any processing. Optionally they can be physically disrupted by mechanical forces such as chopping, grinding, sonicating, pressing, or exposure to vacuum. In another embodiment, the cells can be disrupted by exposure to freezing or exposure to high temperatures. Alternatively, they can lysed by the addition of chemical compounds. In another embodiment they can be lysed by the addition of enzymes such as, for example, cellulose and/or alpha glucoidase. In yet another embodiment, they can be genetically modified to produce enzymes that result in the lysis of their own cells upon the administration of some eliciting signal. Such eliciting signals can include exposure to specific temperatures (e.g., use of heat or cold inducible promoters) or exposure to chemical elicitors (e.g., use of chemical inducible promoters such as the dex or tet systems).

Methods are also provided for converting a feedstock into one or more biofuels. In exemplary methods for the processing of a feedstock, a feedstock material as described above is converted by employing one or more enzymes as encoded by SEQ ID NOs: 1-40 and/or one or more enzymes as described in Table 1 under experimental conditions as described herein resulting in a biofuel. For example, a feedstock may be converted to sugars which may be fermented to produce a biofuel (e.g., ethanol). Additionally or alternatively, oils may be extracted from a feedstock which may be converted to a biofuel (e.g., biodiesel).

Feedstock constituents useful for biofuel production include lipids, simple sugars, and complex carbohydrates. More specifically these include glucose, fructose, xylose, monosaccharides, sucrose, disaccharides, starch, cellulose, hemi-cellulose, fructans, xyloologosaccharides, lignin, pectin and triglycerides.

### Business Methods

The present disclosure provides methods for doing business by processing a feedstock with an additive organism. These methods may generate, including, increase, revenue from a biofuel manufacturing process. A biofuel manufacturing business may generate revenue by mixing a feedstock with an additive organism that comprises one or more transgenes present in a minichromosome coding for one or more enzymes, converting the feedstock into sugars and optionally selling the sugars, or alternatively fermenting the sugars to produce a biofuel and selling the biofuel. The sugars and biofuels that are produced by the presently disclosed methods may be sold to a buyer, including, for example, a supplier, a distributor, a manufacturer, a dealer, a reseller, a wholesaler, a retailer or a consumer.

A biofuel manufacturing may also generate, including increase, revenue by using waste products generated from the production process to grow an additive organism. For example, growth of an additive organism, for example, a small aquatic plant may use waste carbon dioxide produced by biofuels fermentation of sugars into ethanol.

Methods of the present disclosure may also generate revenue by reducing, including eliminating, costs associated with processing a feedstock. Costs associated with processing a feedstock (e.g., utilities for heating, steam production, cooling; chemicals for processing, catalysis and neutralization; and/or disposal of toxic waste) may be reduced by producing enzymes in large quantities within an additive organism (e.g., a transgenic plant) and mixing the additive organism with a feedstock. Such methods may reduce, including eliminate, the need for a treatment step to make plant constituents assessable. Further, costs for enzyme production (including, for example, the capital costs of equipment, such as fermentors, the costs of sugars needed to produce enzymes in the equipment, such as fermentors, and the operating costs associated with running and maintaining the equipment, such as fermentors), and the costs for enzyme purification may be reduced since the additive organism is added directly to the biofuels process. Additionally, additive organisms that are plants or other autotrophs can naturally receive their energy source thereby reducing costs associated with a biofuels manufacturing process.

Transportation costs may be reduced if the additive organism is grown in close proximity to the processing plant. Growing plants in containment offers an opportunity for a reduced regulatory process and provides additional markets in countries that may not approve genetically modified crops.

The following examples are included to illustrate embodiments of the disclosure. It should be appreciated by those of skill in the art that the techniques disclosed in the examples that follow represent techniques discovered by the inventors to function well in the practice of the disclosure. However, those of skill in the art should, in light of the present disclosure, appreciate that many changes can be made in the specific embodiments which are disclosed and still obtain a like or similar result without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### EXAMPLES

### Example 1

Genes that are useful for manufacturing a biofuel or another hydrocarbon or co-product by converting feedstock into sugars that can be fermented or chemically converted or by extracting oils that may be processed into biodiesel, for example, genes encoding enzymes, can be isolated and cloned into binary vectors using standard cloning techniques (e.g., PCR, restriction endonuclease digestion, ligation etc.) known to those skilled in the molecular biology arts. For example, sequence specific primers can be used to amplify Endoglucanase I (EGI, Cel7B), from Trichoderma reesei/Hypocrea jecorina (GenBank Accession Number M15665) (SEQ ID NO: 2). Alternatively, since the sequence of Endoglucanase I (EGI, Cel7B) is known, a synthetic version of the gene could be produced using commercial services such as GenScript Corporation or Biomatik Corp. or Codon Devices. It is understood that a set of such genes can be grouped into a set. Such sets may be referred to as “gene stacks”. This target sequence(s) can then be incorporated into a plasmid capable of propagation in E. coli and Agrobacterium tumefaciens (binary vector) that also comprises the necessary components (T-DNA left boarder, T-DNA right boarder) for bio-mediated transfer from A. tumefaciens into the plant host genome. Many binary vectors have been described (see, e.g., R. Xu et al. (2008) Plant Methods 4:4; A. Himmelbach et al. (2007) Plant Physiol. 145(4):1192-1200; T. Komori et al. (2007) Plant Physiol. 145: 1155-1160).

The engineering of the target gene(s) into the binary vector typically (but not necessarily) uses standard cloning procedures involving E. coli. Once constructed the binary vector comprising the desired gene(s) is then transformed into Agrobacterium. Agrobacterium can be transformed following the procedures of Weigel and Glazebrook (see, e.g., Weigel D and Glazebrook J. “How to Transform Arabidopsis” in Arabidopsis a Laboratory Manual. Cold Spring Harbor Laboratory Press, Cold Spring Harbor, N.Y. pp. 119-141). Five hundred milliliters of LB media (made by dissolving 10 grams of Bacto Tryptone, 5 grams of Yeast Extract, and five grams of NaCl in 1000 milliliters of sterile water) is inoculated with five milliters of an overnight culture of the appropriate Agrobacterium strain (several can be used including: LBA4404, GV2260, C58C1, GV3101::pMP90, GV3101::pMP90RK and AGL-1). Next, the culture is incubated with vigorous agitation at 28° C. overnight. Once the cells reach mid-log phase (e.g., an OD550 of 0.5 to 0.8) the culture is chilled on ice. Next, the chilled culture is pelleted by centrifugation at 4000 g for ten minutes at 4° C. The supernatant is then discarded and the pellet resuspended in ten milliliters of ice-cold sterile, double-distilled water. Once the cells are resuspended the total volume is brought up to five hundred milliliters using ice-cold sterile, double-distilled water. The centrifugation is then repeated and the pellet washed two more times with 250 milliliters and 50 milliliters on the 2^{nd} and 3^{rd} rinse respectively. The cells are pelleted once more and then resuspended in five milliliters of ice-cold, sterile, 10% (w/v) glycerol. These cells can now be transformed with the engineered binary vector. A fifty microliter aliquot of the transformation competent Agrobacterium cells are mixed with one microliter of binary vector prepared from a standard E. coli mini-prep (see, e.g., Sambrook J and Russell D W “Preparation of Plasmid DNA by Alkaline Lysis with SDS: Minipreparation” in Molecular Cloning a Laboratory Manual, Third Edition, Cold Spring Harbor Laboratory Press, Cold Spring Harbor, N.Y. pp. 1.32-1.37). The cells are mixed and the DNA is placed on ice and then transferred to a electroporation cuvette. Electroporate the sample following manufacturers recommended settings. One milliliter of LB is added to the cuvette following electroporation, and then the cell suspension is transfered to a fifteen milliliter culture tube. Next, the sample is incubated for four hours at 28° C. with gentle agitation. An aliquot of the cells is applied to an LB agar plate containing the appropriate antibiotic (determined by the backbone of the parental binary vector used). The cells are incubated for three to four days at 28° C. The colonies are restraked on fresh LB agar plates, incubated for another three to four days and single colonies selected. Selected colonies may then be used to inoculate a five milliliter liquid LB culture (with appropriate selection).

The liquid culture is grown for three to four hours at 28° C. (vigorous agitation) and one milliliter of this culture is used to inoculate two hundred milliliter LB culture (with appropriate selection). Next, the larger culture is grown overnight at 28° C. with vigorous agitation until the reach mid-log phase (OD₅₅₀ 0.5 to 0.8). The culture is centrifuged at 6000 rpm for ten minutes at room temperature and the cell pellet is resuspended in four hundred milliliters of infiltration media (e.g., 0.5× Murashige and Skoog salts (Sigma), 1× Gamborg's B5 vitamins (GIBCO), 5% (w/v) sucrose, 0.044 benzylamino purine (10 microliters of a 1 milligram/milliliter stock in DMSO) and fifty microliters of Silwet L-77 (from Lehle seeds). This suspension of Agrobacterium can be used to transform plants. Transformation can be achieved by a number of different means that all involve contacting the plant with the Agrobacterium. For example, the plants (including, for example, plant parts or cells) may be sprayed with the suspension, the flowers of the plant may be dipped in the suspension or the plant may be (partially) submerged in the suspension and exposed to vacuum (vacuum infiltration). Plants (including, for example, plant parts or cells) that have been transformed with the Agrobacterium are then allowed to either set seed (in the case of whole plants) or regenerate (in the case of plant parts or cells). Regeneration or selection of transformed seeds can be aided by the application of a selective agent (including, for example, an antibiotic or herbicide, such as kanamycin or glyphosate) corresponding to a resistance gene on the binary vector.

### Example 2

Genes that are useful for manufacturing a biofuel or another hydrocarbon or co-product by converting feedstock into sugars that can be fermented or chemically converted or by extracting oils that may be processed into biodiesel, for example, genes encoding enzymes, can be isolated and delivered via biolistic delivery. A biolistic delivery method using wet gold particles kept in an aqueous DNA suspension adapted from the teachings of Milahe and Miller (Biotechniques 16: 924-931, 1994) is useful for transforming plant cells. To prepare the wet gold particles for bombardment, 1.0 μm gold particles are washed by mixing with 100% ethanol on a vortex followed by spinning the particles in a microfuge at 4000 rpm in order to remove supernatant. Subsequently, the gold particles are washed with sterile distilled water three times, followed by spinning in a microfuge to remove supernatant. The washed gold particles are resuspend in sterile distilled water at a final concentration of 90 mg/ml and stored at 4° C. until use. For bombardment, the gold particle suspension (90 mg/ml) is then mixed rapidly with 1 μg/μl DNA solution (in dH₂O or TE), 2.5M CaCl₂, and 1M spermidine. If two or more plasmids (e.g., comprising the genes desired for transfer into the plant, for example, genes encoding enzymes) are contained within the DNA solution, equal amounts of each plasmid are added to the gold suspension.

Several types of tissue can be used as a target for bombardment (e.g., tissue explants, excised embryos, embryonic callus, etc.). Here tissue explants are described. To prepare explant tissues for DNA delivery, three days prior to bombardment, an internode of the plant is excised. The internode explant is cut longitudinally with a scalpel to cut a thin slice (⅙-¼ of the internode) off one side of the explant. The prepared internodes is placed wound side down on Petri dishes with regeneration media. The Petri dishes were wrapped with tape and placed wound side up under the light. The explants grew for three days prior to bombardment.

For bombardment of suspension cells, the cells are harvested by centrifugation (1200 rpm for two minutes) on the day of bombardment. The cells are plated onto fifty millimeter circular polyester screen cloth disks placed on petri plates with solid medium. The solid medium used is the same medium that the cells are normally grown in (MS salts, Gamborg's vitamins, 3% sucrose, 2 mg/liter 2,4D (2,4-Dichlorophenoxyacetic acid), 0.5 mM MES pH 5.8 +(solid medium only), plus 0.26% gelrite, or 0.6% tissue culture agar, added before autoclaving. Approximately 1.5 ml packed cells are placed on each filter disk, and dispersed uniformly into a very even spot approximately one inch in diameter.

Bombardment of the cells is carried out in the BioRad PDS-1000/He Biolistic Particle Delivery System (BioRad)—though any particle delivery system could be used. The DNA/gold suspension is resuspended and immediately inserted onto the grid of the filter holder. A fifty millimeter circular polyester screen cloth disk containing the cells is placed into a fresh sixty millimeter petri dish and the cells are covered with a 10×10 cm square of sterile nylon or Dacron chiffon netting. The metal cylinder is inserted into the petri dish and used to push the netting down to the bottom of the dish. This weight prevents the cells from being dislodged from the plate during bombardment. The petri dish containing the cells is then placed onto the sample holder, and positioned in the sample chamber of the gene gun and bombarded with the DNA/gold suspension. After the bombardment, the cells are scraped off the filter circle in the petri dish containing solid medium with a sterile spatula and transferred to fresh medium in a one hundred and twenty five milliliter blue-capped glass bottle. The bottles are transferred onto a shaker and grown while shaking at 150 rpm.

A biolistic delivery method using dry gold particles can also carried out to deliver the desired genes into plant cells. For this method, 1.0 or 0.6μ gold particles are washed in 70% ethanol with vigorous shaking on a vortex for three to five minutes, followed by a soaking in 70% ethanol for fifteen minutes. The gold particles are spun in a microfuge to remove the supernatant and washed three times in sterile distilled water. The gold particles are suspended in 50% glycerol at a concentration of 60 mg/ml and stored at 4° C. For bombardment, the dry gold particles are resuspended on a vortex for five minutes to disrupt agglomerated particles. Subsequently, the dry gold particles are mixed rapidly with DNA, 2.5M CaCl₂ and 0.2M spermidine in a siliconized, sterile eppendorf tube. The sample is allowed to settle for one minute and then spun in a microfuge for ten seconds to remove supernatant. Subsequently, the DNA/gold particles are washed once with 70% ethanol, followed by two washed in 100% ethanol. A portion of the DNA/gold mixture is evenly placed on a macrocarrier. The macrocarrier was then placed in the BioRad PDS-1000/He Biolistic Particle Delivery System, and the bombardment was done at rupture disk pressures ranging from 450 psi to 2,200 psi.

While the present disclosure has been described and illustrated herein by references to various specific materials, procedures and examples, it is understood that the disclosure is not restricted to the particular combinations of material and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1-77. (canceled)

78. A method for processing a feedstock, the method comprising:
mixing the feedstock with at least one additive organism that comprises one or more transgenes coding for one or more enzymes.

79. The method of claim 78, further comprising after mixing the feedstock with the additive organism, converting the feedstock into sugars, and fermenting the sugars to produce a biofuel.

80. The method of claim 78, further comprising after mixing the feedstock with the additive organism, extracting oil from the feedstock and converting the oil to produce a biofuel.

81. The method of claim 78 or 79, wherein the biofuel is selected from the group consisting of: ethanol, propanol, butanol, methanol, methane, 2,5-dimethylfuran, dimethyl ether, biodiesel, paraffins and hydrogen.

82. The method of claim 78, wherein the additive organism comprises at least one transgene, optionally operably linked to an inducible promoter, comprising a polynucleotide sequence selected from the group consisting of SEQ ID NOS: 1-40.

83. The method of claim 78, wherein the additive organism comprises multiple transgenes, wherein the multiple transgenes sets are selected from the group of sets:
(a) Set 1: SEQ ID NOS: 1, 19, 27;
(b) Set 2: SEQ ID NOS: 7, 16, 18, 28;
(c) Set 3: SEQ ID NOS: 2, 16, 18, 25;
(d) Set 4: SEQ ID NOS: 3, 5, 16, 18, 25;
(e) Set 5: SEQ ID NOS: 6, 16, 18, 26, 27;
(f) Set 6: SEQ ID NOS: 8, 9, 10, 21, 22, 35; and
(g) Set 7: SEQ ID NOS: 8, 9, 10, 21, 22, 35

84. The method of claim 78, wherein the feedstock is selected from the group consisting of: lignocellulosic material, recycled materials, forestry waste, industrial waste materials, livestock waste, and municipal wastes, oilseeds, algae, animal waste and vegetable oil.

85. The method of claim 78, further comprising treatment of the feedstock, wherein the additive organism is added before or after treatment of the feedstock.

86. The method of claim 85, wherein the treatment comprises a thermo or chemical component.

87. The method of claim 85, therein the treatment is selected from the group consisting of: heat at 140° C. to 200° C., steam explosion, ammonia fiber explosion, acid or alkaline treatment, and physical disruption.

88. The method of claim 87, wherein the physical disruption is at least one selected from the group consisting of chopping, grinding, sonicating, pressing, exposure to vacuum, exposure freezing, exposure to high temperature, exposure to chemical compounds, and exposure to enzymes.

89. The method of 88, wherein the exposure to enzymes comprises exposing the feedstock to a cellulase or an alpha glucosidase.

90. The method of claim 78, wherein the additive organism is selected from the group consisting of a plant, an alga, and a fungus.

91. The method of claim 90, wherein the additive organism is an alga and is selected from the group consisting of Lemna minor, Chlamdomonas reinhardii, Agrobacterium Chlamydomonas, Dunaliella or Chlorella.

92. The method of claim 78, wherein the additive organism is modified to produce one or more enzymes that result in the lysis of the cells of the additive organism upon the administration of an eliciting signal.

93. The method of claim 92, wherein the enzyme performs at least one function selected from the group consisting of (1) breaking down glucans, xyloglucans, xylans, mannans; (2) cell wall components; (3) removing one or more inhibitors of fermentation; and (4) improving fermentation.

94. The method of claim 93, wherein the enzyme breaks down glucan, xyloglucans, xylans, or mannans and is selected from the group consisting of: endo-β(1,4)-glucanase, cellobiohydrolase, β-glucosidase, α-/β-glucosidase, mixed-linked glucanase, endo-β(1,3)-glucanase, exo-β(1,3)-glucanase, β-(1,6)-glucanase, hemi-cellulases/xylanases, endo-1,4-β-xylanases, β-xylosidases, glycosyl hydrolase, α-L-arabinofuranosidases, α-glucuronidases, xyloglucan-specific endoglucanase, oligoxyloglucan reducing end-specific xyloglucanase, α-fucosidase, α-xylosidase, endo-β(1,4)-xylanase, β-xylosidase, β-xylosidase/α-arabinosidase, acetylxylan esterase, ferulic acid esterase, a glucuronidase, endo-β(1,4)-mannanase, β-mannosidase, α-galactosidase.

95. The method of claim 93, wherein the enzyme breaks down cell wall components and is selected from the group consisting of: ligninases, acetylesterases, pectinases, pectin lyase, pectate lyase, endo-polygalacturonase, exo-polygalacturonase, pectin methyl esterase, rhamnogalacturonase, rhamnogalacturonan lyase, rhamnogalacturonan acetylesterase, α-L-rhamnosidase, endo-α(1,5)- [symbol] rabinosidase, α-L-arabinofuranosidase, endo-β(1,4)-galactanase, xylogalacturonase and β-galactosidase.

96. An additive organism for processing a feedstock, said organism comprising one or more transgenes coding for one or more enzymes, wherein the enzymes are encoded by one or more polynucleotides selected from the group consisting of SEQ ID NOS: 1-40.
